(19) United States
(12) Patent Application Publication
(10) Pub. No.: US 2007/0067027 A1
(43) Pub. Date: Mar. 22, 2007

(54) Left atrial balloon catheter

(75) Inventors: Shahram (Shawn) Moaddeb, Irvine, CA (US); Richard Rhee, Anaheim, CA (US); Steve Anderson, Rancho Santa Margarita, CA (US)

Correspondence Address: MCDERMOTT WILL & EMERY LLP, 18191 VON KARMAN AVE., SUITE 500, IRVINE, CA, 92612-7108, US

(73) Assignee: Micardia Corporation, Irvine, CA (US)

(21) Appl. No.: 11/520,981

(22) Filed: Sep. 13, 2006

Related U.S. Application Data
(60) Provisional application No. 60/717,112, filed on Sep. 14, 2005.

(51) Int. Cl. A61F 2/24

(52) U.S. Cl. 623/2.11; 623/2.37

(57) ABSTRACT

Systems, methods and devices are provided for activation of an adjustable annuloplasty device. The devices may include a catheter system for percutaneously activating an adjustable annuloplasty device, including a handle assembly, a shaft assembly having at least one fluid lumen, and a distal element. The shaft assembly extends between the handle assembly and the distal element, the distal element being in fluid communication with the handle assembly via the at least one fluid lumen. The distal element includes an elongated core having a first port and an expandable member. The core extends through the expandable member and the expandable member is movable between a collapsed position and an inflated position. The distal element has a preset shape in the inflated position, having a long axis that is curvilinear. A surface of the distal element extends along the long axis and is configured to conform to a curvilinear surface of the annuloplasty device. In some arrangements, the annuloplasty device includes a ring, and the circumference of the annuloplasty device is a circumference of the ring.

### RELATED APPLICATIONS

This application claims the benefit under 35 U.S.C. § 119(e) of U.S. Provisional Application No. 60/717,112, filed Sep. 14, 2005, the entirety of which is hereby incorporated by reference herein.

### BACKGROUND OF THE INVENTION

1. Field of the Invention

The present invention relates to methods and devices for implantable devices. More specifically, the present invention relates to catheter systems that can activate and change the configurations of implantable devices.

2. Description of the Related Art

The circulatory system of mammals includes the heart and the interconnecting vessels throughout the body that include both veins and arteries. The human heart includes four chambers, which are the left and right atrium and the left and right ventricles. The mitral valve, which allows blood flow in one direction, is positioned between the left ventricle and left atrium. The tricuspid valve is positioned between the right ventricle and the right atrium. The aortic valve is positioned between the left ventricle and the aorta, and the pulmonary valve is positioned between the right ventricle and pulmonary artery. The heart valves function in concert to move blood throughout the circulatory system. The right ventricle pumps oxygen-poor blood from the body to the lungs and then into the left atrium. From the left atrium, the blood is pumped into the left ventricle and then out the aortic valve into the aorta. The blood is then recirculated throughout the tissues and organs of the body and returns once again to the right atrium.

If the valves of the heart do not function properly, due either to disease or congenital defects, the circulation of the blood may be compromised. Diseased heart valves may be stenotic, wherein the valve does not open sufficiently to allow adequate forward flow of blood through the valve, and/or incompetent, wherein the valve does not close completely. Incompetent heart valves cause regurgitation or excessive backward flow of blood through the valve when the valve is closed. For example, certain diseases of the heart valves can result in dilation of the heart and one or more heart valves. When a heart valve annulus dilates, the valve leaflet geometry deforms and causes ineffective closure of the valve leaflets. The ineffective closure of the valve can cause regurgitation of the blood, accumulation of blood in the heart, and other problems.

Diseased or damaged heart valves can be treated by valve replacement surgery, in which damaged leaflets are excised and the annulus is sculpted to receive a replacement valve. Another repair technique that has been shown to be effective in treating incompetence is annuloplasty, in which the effective size of the valve annulus is contracted by attaching a prosthetic annuloplasty repair segment or ring to an interior wall of the heart around the valve annulus. The annuloplasty ring reinforces the functional changes that occur during the cardiac cycle to improve coaptation and valve integrity. Thus, annuloplasty rings help reduce reverse flow or regurgitation while permitting good hemodynamics during forward flow.

Generally, annuloplasty rings comprise an inner substrate of a metal such as stainless steel or titanium, or a flexible material such as silicon rubber or Dacron®. The inner substrate is generally covered with a biocompatible fabric or cloth to allow the ring to be sutured to the heart tissue. Annuloplasty rings may be stiff or flexible, may be open or closed, and may have a variety of shapes including circular, D-shaped, or C-shaped. The configuration of the ring is generally based on the shape of the heart valve being repaired or on the particular application. For example, the tricuspid valve is generally circular and the mitral valve is generally D-shaped. Further, C-shaped rings may be used for tricuspid valve repairs, for example, because it allows a surgeon to position the break in the ring adjacent the atrioventricular node, thus avoiding the need for suturing at that location.

Annuloplasty rings support the heart valve annulus and restore the valve geometry and function. Although the implantation of an annuloplasty ring can be effective, the heart of a patient may change geometry over time after implantation. For example, the heart of a child will grow as the child ages. As another example, after implantation of an annuloplasty ring, dilation of the heart caused by accumulation of blood may cease and the heart may begin returning to its normal size. Whether the size of the heart grows or reduces after implantation of an annuloplasty ring, the ring may no longer be the appropriate size for the changed size of the valve annulus.

### SUMMARY OF THE INVENTION

Thus, it would be advantageous to develop systems and methods for reinforcing a heart valve annulus or other body structure using an annuloplasty device that can be adjusted within the body of a patient in a minimally invasive or non-invasive manner. In an embodiment, a method for treating a cardiac valve is provided. The method includes providing an annuloplasty ring having a first size of a dimension in a first configuration and a second size of the dimension in a second configuration, wherein the second size is less than the first size in the septal-lateral distance (or anterior/posterior distance). The method further includes attaching the annuloplasty ring while in the first configuration to or near a valve annulus in a heart having a first end-diastolic volume of a ventricle. After the ventricle has a second end-diastolic volume, the second end-diastolic volume being less than the first end-diastolic volume, the method includes changing the annuloplasty ring from the first configuration to the second configuration.

In some embodiments, a catheter system for percutaneously activating an adjustable annuloplasty device, the catheter system comprising: a handle assembly; a shaft assembly having at least one fluid lumen; and a distal element, the shaft assembly extends between the handle assembly and the distal element, the distal element being in fluid communication with the handle assembly via the at least one fluid lumen, the distal element comprising: an elongated core having a first port; an expandable member, the core extending through the expandable member, the expandable member being movable between a collapsed position and an inflated position, wherein the distal element has a preset shape, in the inflated position, having a long axis that is curvilinear, and a surface of the distal element extending along the long axis is configured to conform to a curvilinear surface of the annuloplasty device, said annuloplasty device surface extending along a circumference of the annuloplasty device. In some arrangements, the annuloplasty device comprises a ring, and the circumference of the annuloplasty device is a circumference of the ring.

In some embodiments, a system for activating a device implanted in a patient is provided. The system comprising: a handle assembly; a flexible, steerable shaft assembly; and a distal element being expandable between a first position and a second position, the distal element being dimensioned so as to have a curvilinear long axis that matches a curvilinear long axis of an annuloplasty device implanted at or near a valve in a patient's heart.

In some embodiments, a method for activating an implantable device is provided. The method comprises: providing a catheter assembly having a distal element thereon; positioning the distal element within an atrium of a heart of a patient proximal to an annuloplasty device located at or near a valve of said heart; and delivering sufficient energy from said distal element to said annuloplasty device to change a configuration of said annuloplasty device.

### BRIEF DESCRIPTION OF THE DRAWINGS

Systems and methods which embody the various features of the invention will now be described with reference to the following drawings:

FIG. 1A is a top view in partial section of an adjustable annuloplasty ring according to certain embodiments of the invention;

FIG. 1B is a side view of the annuloplasty ring of FIG. 1A;

FIG. 1C is a transverse cross-sectional view of the annuloplasty ring of FIG. 1A;

FIG. 2 is a graphical representation of the diameter of an annuloplasty ring in relation to the temperature of the annuloplasty ring according to certain embodiments of the invention;

FIG. 3A is a top view in partial section of an adjustable annuloplasty ring having a D-shaped configuration according to certain embodiments of the invention;

FIG. 3B is a side view of the annuloplasty ring of FIG. 3A;

FIG. 3C is a transverse cross-sectional view of the annuloplasty ring of FIG. 3A;

FIG. 4A is a top view of an annuloplasty ring having a substantially circular configuration according to certain embodiments of the invention;

FIG. 4B is a side view of the annuloplasty ring of FIG. 4A;

FIG. 4C is a transverse cross-sectional view of the annuloplasty ring of FIG. 4A;

FIG. 5 is a top view of an annuloplasty ring having a substantially D-shaped configuration according to certain embodiments of the invention;

FIG. 6A is a schematic diagram of a top view of a shape memory wire having a substantially D-shaped configuration according to certain embodiments of the invention;

FIGS. 6B-6E are schematic diagrams of side views of the shape memory wire of FIG. 6A according to certain embodiments of the invention;

FIG. 7A is a perspective view in partial section of an annuloplasty ring comprising the shape memory wire of FIG. 6A according to certain embodiments of the invention;

FIG. 7B is a perspective view in partial section of a portion of the annuloplasty ring of FIG. 7A;

FIG. 8 is a schematic diagram of a shape memory wire having a substantially C-shaped configuration according to certain embodiments of the invention;

FIG. 9A is a perspective view in partial section of an annuloplasty ring comprising the shape memory wire of FIG. 8 according to certain embodiments of the invention;

FIG. 9B is a perspective view in partial section of a portion of the annuloplasty ring of FIG. 9A;

FIG. 10A is a perspective view in partial section an annuloplasty ring comprising a first shape memory wire and a second shape memory wire according to certain embodiments of the invention;

FIG. 10B is a top cross-sectional view of the annuloplasty ring of FIG. 10A;

FIG. 11A is a perspective view in partial section of an annuloplasty ring comprising a first shape memory wire and a second shape memory wire according to certain embodiments of the invention;

FIG. 11B is a top cross-sectional view of the annuloplasty ring of FIG. 11A;

FIG. 12 is a perspective view of a shape memory wire wrapped in a coil according to certain embodiments of the invention;

FIGS. 13A and 13B are schematic diagrams illustrating an annuloplasty ring according to certain embodiments of the invention;

FIG. 14 is a schematic diagram illustrating an annuloplasty ring according to certain embodiments of the invention;

FIG. 15 is a schematic diagram illustrating an annuloplasty ring according to certain embodiments of the invention;

FIGS. 16A and 16B are schematic diagrams illustrating an annuloplasty ring having a plurality of temperature response zones or sections according to certain embodiments of the invention;

FIGS. 17A and 17B are schematic diagrams illustrating an annuloplasty ring having a plurality of temperature response zones or sections according to certain embodiments of the invention;

FIG. 18 is a sectional view of a mitral valve with respect to an exemplary annuloplasty ring according to certain embodiments of the invention;

FIG. 19 is a schematic diagram of a substantially C-shaped wire comprising a shape memory material configured to contract in a first direction and expand in a second direction according to certain embodiments of the invention;

FIGS. 20A and 20B are schematic diagrams of a body member usable by an annuloplasty ring according to certain embodiments of the invention;

FIGS. 21A and 21B are schematic diagrams of a body member usable by an annuloplasty ring according to certain embodiments of the invention;

FIGS. 22A and 22B are schematic diagrams of a body member usable by an annuloplasty ring according to certain embodiments of the invention;

FIG. 23 is a transverse cross-sectional view of the body member of FIGS. 21A and 21B;

FIG. 24 is a perspective view of a body member usable by an annuloplasty ring according to certain embodiments comprising a first shape memory band and a second shape memory band;

FIG. 25A is a schematic diagram illustrating the body member of FIG. 24 in a first configuration or shape according to certain embodiments of the invention;

FIG. 25B is a schematic diagram illustrating the body member of FIG. 24 in a second configuration or shape according to certain embodiments of the invention;

FIG. 25C is a schematic diagram illustrating the body member of FIG. 24 in a third configuration or shape according to certain embodiments of the invention;

FIG. 26 is a perspective view illustrating an annuloplasty ring comprising one or more thermal conductors according to certain embodiments of the invention;

FIGS. 27A-27C are transverse cross-sectional views of the annuloplasty ring of FIG. 26 schematically illustrating exemplary embodiments of the invention for conducting thermal energy to an internal shape memory wire; and

FIG. 28 is a schematic diagram of an annuloplasty ring comprising one or more thermal conductors according to certain embodiments of the invention.

FIG. 29 is a cross-sectional view of the patient's heart with a catheter system placed therein, a distal element of the catheter system is in operative engagement with an implanted device;

FIG. 29A is a cross-sectional top view of the implanted device and associated mitral valve of the heart of FIG. 29;

FIG. 29B illustrates a distal element of the catheter system positioned over the implantable device of FIG. 29A;

FIG. 30A is a perspective view of a catheter system configured to activate an implantable device, the catheter system has a handle assembly in a first position;

FIG. 30B is a perspective view of the catheter system of FIG. 30A, wherein the handle assembly is in a second position;

FIG. 31 is a side elevational view of the catheter system of FIG. 30A, wherein the distal element is moved between a first position and a second position;

FIG. 32 is a longitudinal cross-sectional view of the catheter system of FIG. 31;

FIG. 32A is an end view of the distal element of the catheter system of FIG. 32;

FIG. 32B is a close-up view of the core portion of the distal element of FIG. 32A;

FIG. 33 is an enlarged perspective view of the distal element of the catheter system of FIG. 30A;

FIG. 34 is a top plan view of the distal element of the catheter system of FIG. 30A;

FIG. 35 is a transverse cross-sectional view of the distal element of the catheter system of FIG. 30A, the distal element is in operative engagement with an implantable device in situ;

FIG. 36 is a top plan view of the distal element in accordance with another embodiment;

FIG. 37 is a cross-sectional view of the distal element of FIG. 36 taken along the line 37-37;

FIG. 38 is a top plan view of the distal element in accordance with another embodiment;

FIG. 39 is a side elevational view of the distal element of FIG. 38;

FIG. 40A is a cross-sectional view of a distal element engagement with an implantable device, the distal element has a structure for positioning the distal element;

FIG. 40B illustrates another embodiment of a distal element having an alignment structure configured to mate with an implantable device;

FIG. 41 is a longitudinal cross-sectional view of a portion of the catheter system of FIG. 30A;

FIG. 42 is a cross-sectional view of the catheter system taken along the line 42-42 of FIG. 41;

FIG. 43 is an enlarged cross-sectional view of the handle assembly of the catheter system of FIG. 30A;

FIG. 44 illustrates a delivery sheath positioned within a patient's heart, the catheter system is being advanced through the delivery sheath;

FIG. 45 illustrates a distal element of the catheter system passing out of the delivery sheath;

FIG. 46 illustrates the distal element positioned within a left atrium of the heart;

FIG. 47 is a perspective view of a catheter system in accordance with another embodiment;

FIG. 48 is a side perspective view of the catheter system of FIG. 49;

FIG. 49 is a cross-sectional view of a distal element in accordance with another embodiment;

FIG. 50 is a cross-sectional view of the distal element of FIG. 49 in engagement with an implantable device;

FIG. 51 is a cross-sectional view of distal element in accordance with another embodiment, the distal element is in a first position;

FIG. 52 is a cross-sectional view of distal element of FIG. 51, the distal element is in a second position; and

FIG. 53 is a cross-sectional view of the distal element of FIG. 51 in a neutral position.

### DETAILED DESCRIPTION OF THE PREFERRED EMBODIMENT

The present invention involves systems and methods for reinforcing dysfunctional heart valves and other body structures with adjustable rings. In certain embodiments, an adjustable annuloplasty ring is implanted into the body of a patient such as a human or other animal. The adjustable annuloplasty ring is implanted through an incision or body opening either thoracically (e.g., open-heart surgery) or percutaneously (e.g., via a femoral artery or vein, or other arteries or veins) as is known to someone skilled in the art. The adjustable annuloplasty ring is attached to the annulus of a heart valve to improve leaflet coaptation and to reduce regurgitation. The annuloplasty ring may be selected from one or more shapes comprising a round or circular shape, an oval shape, a C-shape, a D-shape, a U-shape, an open circle shape, an open oval shape, and other curvilinear shapes.

The size of the annuloplasty ring can be adjusted postoperatively to compensate for changes in the size of the heart. As used herein, the term “postoperatively” refers to a time after implanting the adjustable annuloplasty ring and closing the body opening through which the adjustable annuloplasty ring was introduced into the patient's body. For example, the annuloplasty ring may be implanted in a child whose heart grows as the child gets older. Thus, the size of the annuloplasty ring may need to be increased. As another example, the size of an enlarged heart may start to return to its normal size after an annuloplasty ring is implanted. Thus, the size of the annuloplasty ring may need to be decreased postoperatively to continue to reinforce the heart valve annulus.

In certain embodiments, the annuloplasty ring comprises a shape memory material that is responsive to changes in temperature and/or exposure to a magnetic field. Shape memory is the ability of a material to regain its shape after deformation. Shape memory materials include polymers, metals, metal alloys and ferromagnetic alloys. The annuloplasty ring is adjusted in vivo by applying an energy source to activate the shape memory material and cause it to change to a memorized shape. The energy source may include, for example, radio frequency (RF) energy, x-ray energy, microwave energy, ultrasonic energy such as focused ultrasound, high intensity focused ultrasound (HIFU) energy, light energy, electric field energy, magnetic field energy, combinations of the foregoing, or the like. For example, one embodiment of electromagnetic radiation that is useful is infrared energy having a wavelength in a range between approximately 750 nanometers and approximately 1600 nanometers. This type of infrared radiation may be produced efficiently by a solid state diode laser. In certain embodiments, the annuloplasty ring implant is selectively heated using short pulses of energy having an on and off period between each cycle. The energy pulses provide segmental heating which allows segmental adjustment of portions of the annuloplasty ring without adjusting the entire implant.

In certain embodiments, the annuloplasty ring includes an energy absorbing material to increase heating efficiency and localize heating in the area of the shape memory material. Thus, damage to the surrounding tissue is reduced or minimized. Energy absorbing materials for light or laser activation energy may include nanoshells, nanospheres and the like, particularly where infrared laser energy is used to energize the material. Such nanoparticles may be made from a dielectric, such as silica, coated with an ultra thin layer of a conductor, such as gold, and be selectively tuned to absorb a particular frequency of electromagnetic radiation. In certain such embodiments, the nanoparticles range in size between about 5 nanometers and about 20 nanometers and can be suspended in a suitable material or solution, such as saline solution. Coatings comprising nanotubes or nanoparticles can also be used to absorb energy from, for example, HIFU, MRI, inductive heating, or the like.

In other embodiments, thin film deposition or other coating techniques such as sputtering, reactive sputtering, metal ion implantation, physical vapor deposition, and chemical deposition can be used to cover portions or all of the annuloplasty ring. Such coatings can be either solid or microporous. When HIFU energy is used, for example, a microporous structure traps and directs the HIFU energy toward the shape memory material. The coating improves thermal conduction and heat removal. In certain embodiments, the coating also enhances radio-opacity of the annuloplasty ring implant. Coating materials can be selected from various groups of biocompatible organic or non-organic, metallic or non-metallic materials such as Titanium Nitride (TiN), Iridium Oxide (Irox), Carbon, Platinum black, Titanium Carbide (TiC) and other materials used for pacemaker electrodes or implantable pacemaker leads. Other materials discussed herein or known in the art can also be used to absorb energy.

In addition, or in other embodiments, fine conductive wires such as platinum coated copper, titanium, tantalum, stainless steel, gold, or the like, are wrapped around the shape memory material to allow focused and rapid heating of the shape memory material while reducing undesired heating of surrounding tissues.

In certain embodiments, the energy source is applied surgically either during implantation or at a later time. For example, the shape memory material can be heated during implantation of the annuloplasty ring by touching the annuloplasty ring with warm object. As another example, the energy source can be surgically applied after the annuloplasty ring has been implanted by percutaneously inserting a catheter into the patient's body and applying the energy through the catheter. For example, RF energy, light energy or thermal energy (e.g., from a heating element using resistance heating) can be transferred to the shape memory material through a catheter positioned on or near the shape memory material. Alternatively, thermal energy can be provided to the shape memory material by injecting a heated fluid through a catheter or circulating the heated fluid in a balloon through the catheter placed in close proximity to the shape memory material. As another example, the shape memory material can be coated with a photodynamic absorbing material which is activated to heat the shape memory material when illuminated by light from a laser diode or directed to the coating through fiber optic elements in a catheter. In certain such embodiments, the photodynamic absorbing material includes one or more drugs that are released when illuminated by the laser light.

In certain embodiments, a removable subcutaneous electrode or coil couples energy from a dedicated activation unit. In certain such embodiments, the removable subcutaneous electrode provides telemetry and power transmission between the system and the annuloplasty ring. The subcutaneous removable electrode allows more efficient coupling of energy to the implant with minimum or reduced power loss. In certain embodiments, the subcutaneous energy is delivered via inductive coupling.

In other embodiments, the energy source is applied in a non-invasive manner from outside the patient's body. In certain such embodiments, the external energy source is focused to provide directional heating to the shape memory material so as to reduce or minimize damage to the surrounding tissue. For example, in certain embodiments, a handheld or portable device comprising an electrically conductive coil generates an electromagnetic field that non-invasively penetrates the patient's body and induces a current in the annuloplasty ring. The current heats the annuloplasty ring and causes the shape memory material to transform to a memorized shape. In certain such embodiments, the annuloplasty ring also comprises an electrically conductive coil wrapped around or embedded in the memory shape material. The externally generated electromagnetic field induces a current in the annuloplasty ring's coil, causing it to heat and transfer thermal energy to the shape memory material.

In certain other embodiments, an external HIFU transducer focuses ultrasound energy onto the implanted annuloplasty ring to heat the shape memory material. In certain such embodiments, the external HIFU transducer is a handheld or portable device. The terms “HIFU,” “high intensity focused ultrasound” or “focused ultrasound” as used herein are broad terms and are used at least in their ordinary sense and include, without limitation, acoustic energy within a wide range of intensities and/or frequencies. For example, HIFU includes acoustic energy focused in a region, or focal zone, having an intensity and/or frequency that is considerably less than what is currently used for ablation in medical procedures. Thus, in certain such embodiments, the focused ultrasound is not destructive to the patient's cardiac tissue. In certain embodiments, HIFU includes acoustic energy within a frequency range of approximately 0.5 MHz and approximately 30 MHz and a power density within a range of approximately 1 W/cm² and approximately 500 W/cm².

In certain embodiments, the annuloplasty ring comprises an ultrasound absorbing material or hydro-gel material that allows focused and rapid heating when exposed to the ultrasound energy and transfers thermal energy to the shape memory material. In certain embodiments, a HIFU probe is used with an adaptive lens to compensate for heart and respiration movement. The adaptive lens has multiple focal point adjustments. In certain embodiments, a HIFU probe with adaptive capabilities comprises a phased array or linear configuration. In certain embodiments, an external HIFU probe comprises a lens configured to be placed between a patient's ribs to improve acoustic window penetration and reduce or minimize issues and challenges regarding passing through bones. In certain embodiments, HIFU energy is synchronized with an ultrasound imaging device to allow visualization of the annuloplasty ring implant during HIFU activation. In addition, or in other embodiments, ultrasound imaging is used to non-invasively monitor the temperature of tissue surrounding the annuloplasty ring by using principles of speed of sound shift and changes to tissue thermal expansion.

In certain embodiments, non-invasive energy is applied to the implanted annuloplasty ring using a Magnetic Resonance Imaging (MRI) device. In certain such embodiments, the shape memory material is activated by a constant magnetic field generated by the MRI device. In addition, or in other embodiments, the MRI device generates RF pulses that induce current in the annuloplasty ring and heat the shape memory material. The annuloplasty ring can include one or more coils and/or MRI energy absorbing material to increase the efficiency and directionality of the heating. Suitable energy absorbing materials for magnetic activation energy include particulates of ferromagnetic material. Suitable energy absorbing materials for RF energy include ferrite materials as well as other materials configured to absorb RF energy at resonant frequencies thereof.

In certain embodiments, the MRI device is used to determine the size of the implanted annuloplasty ring before, during and/or after the shape memory material is activated. In certain such embodiments, the MRI device generates RF pulses at a first frequency to heat the shape memory material and at a second frequency to image the implanted annuloplasty ring. Thus, the size of the annuloplasty ring can be measured without heating the ring. In certain such embodiments, an MRI energy absorbing material heats sufficiently to activate the shape memory material when exposed to the first frequency and does not substantially heat when exposed to the second frequency. Other imaging techniques known in the art can also be used to determine the size of the implanted ring including, for example, ultrasound imaging, computed tomography (CT) scanning, X-ray imaging, or the like. In certain embodiments, such imaging techniques also provide sufficient energy to activate the shape memory material.

In certain embodiments, imaging and resizing of the annuloplasty ring is performed as a separate procedure at some point after the annuloplasty ring as been surgically implanted into the patient's heart and the patient's heart, pericardium and chest have been surgically closed. However, in certain other embodiments, it is advantageous to perform the imaging after the heart and/or pericardium have been closed, but before closing the patient's chest, to check for leakage or the amount of regurgitation. If the amount of regurgitation remains excessive after the annuloplasty ring has been implanted, energy from the imaging device (or from another source as discussed herein) can be applied to the shape memory material so as to at least partially contract the annuloplasty ring and reduce regurgitation to an acceptable level. Thus, the success of the surgery can be checked and corrections can be made, if necessary, before closing the patient's chest.

In certain embodiments, activation of the shape memory material is synchronized with the heart beat during an imaging procedure. For example, an imaging technique can be used to focus HIFU energy onto an annuloplasty ring in a patient's body during a portion of the cardiac cycle. As the heart beats, the annuloplasty ring may move in and out of this area of focused energy. To reduce damage to the surrounding tissue, the patient's body is exposed to the HIFU energy only during portions of the cardiac cycle that focus the HIFU energy onto the cardiac ring. In certain embodiments, the energy is gated with a signal that represents the cardiac cycle such as an electrocardiogram signal. In certain such embodiments, the synchronization and gating is configured to allow delivery of energy to the shape memory materials at specific times during the cardiac cycle to avoid or reduce the likelihood of causing arrhythmia or fibrillation during vulnerable periods. For example, the energy can be gated so as to only expose the patient's heart to the energy during the T wave of the electrocardiogram signal.

As discussed above, shape memory materials include, for example, polymers, metals, and metal alloys including ferromagnetic alloys. Exemplary shape memory polymers that are usable for certain embodiments of the present invention are disclosed by Langer, et al. in U.S. Pat. No. 6,720,402, issued Apr. 13, 2004, U.S. Pat. Nos. 6,388,043, issued May 14, 2002, and 6,160,084, issued Dec. 12, 2000, each of which are hereby incorporated by reference herein. Shape memory polymers respond to changes in temperature by changing to one or more permanent or memorized shapes. In certain embodiments, the shape memory polymer is heated to a temperature between approximately 38 degrees Celsius and approximately 60 degrees Celsius. In certain other embodiments, the shape memory polymer is heated to a temperature in a range between approximately 40 degrees Celsius and approximately 55 degrees Celsius. In certain embodiments, the shape memory polymer has a two-way shape memory effect wherein the shape memory polymer is heated to change it to a first memorized shape and cooled to change it to a second memorized shape. The shape memory polymer can be cooled, for example, by inserting or circulating a cooled fluid through a catheter.

Shape memory polymers implanted in a patient's body can be heated non-invasively using, for example, external light energy sources such as infrared, near-infrared, ultraviolet, microwave and/or visible light sources. Preferably, the light energy is selected to increase absorption by the shape memory polymer and reduce absorption by the surrounding tissue. Thus, damage to the tissue surrounding the shape memory polymer is reduced when the shape memory polymer is heated to change its shape. In other embodiments, the shape memory polymer comprises gas bubbles or bubble containing liquids such as fluorocarbons and is heated by inducing a cavitation effect in the gas/liquid when exposed to HIFU energy. In other embodiments, the shape memory polymer may be heated using electromagnetic fields and may be coated with a material that absorbs electromagnetic fields.

Certain metal alloys have shape memory qualities and respond to changes in temperature and/or exposure to magnetic fields. Exemplary shape memory alloys that respond to changes in temperature include titanium-nickel, copper-zinc-aluminum, copper-aluminum-nickel, iron-manganese-silicon, iron-nickel-aluminum, gold-cadmium, combinations of the foregoing, and the like. In certain embodiments, the shape memory alloy comprises a biocompatible material such as a titanium-nickel alloy.

Shape memory alloys exist in two distinct solid phases called martensite and austenite. The martensite phase is relatively soft and easily deformed, whereas the austenite phase is relatively stronger and less easily deformed. For example, shape memory alloys enter the austenite phase at a relatively high temperature and the martensite phase at a relatively low temperature. Shape memory alloys begin transforming to the martensite phase at a start temperature (Mₛ) and finish transforming to the martensite phase at a finish temperature (M_{f}). Similarly, such shape memory alloys begin transforming to the austenite phase at a start temperature (As) and finish transforming to the austenite phase at a finish temperature (A_{f}). Both transformations have a hysteresis. Thus, the Ms temperature and the A_{f} temperature are not coincident with each other, and the Mf temperature and the Aₛ temperature are not coincident with each other.

In certain embodiments, the shape memory alloy is processed to form a memorized shape in the austenite phase in the form of a ring or partial ring. The shape memory alloy is then cooled below the M_{f} temperature to enter the martensite phase and deformed into a larger or smaller ring. For example, in certain embodiments, the shape memory alloy is formed into a ring or partial ring that is larger than the memorized shape but still small enough to improve leaflet coaptation and reduce regurgitation in a heart valve upon being attached to the heart valve annulus. In certain such embodiments, the shape memory alloy is sufficiently malleable in the martensite phase to allow a user such as a physician to adjust the circumference of the ring in the martensite phase by hand to achieve a desired fit for a particular heart valve annulus. After the ring is attached to the heart valve annulus, the circumference of the ring can be adjusted non-invasively by heating the shape memory alloy to an activation temperature (e.g., temperatures ranging from the As temperature to the A_{f} temperature).

Thereafter, when the shape memory alloy is exposed to a temperature elevation and transformed to the austenite phase, the alloy changes in shape from the deformed shape to the memorized shape. Activation temperatures at which the shape memory alloy causes the shape of the annuloplasty ring to change shape can be selected and built into the annuloplasty ring such that collateral damage is reduced or eliminated in tissue adjacent the annuloplasty ring during the activation process. Exemplary A_{f} temperatures for suitable shape memory alloys range between approximately 45 degrees Celsius and approximately 70 degrees Celsius. Furthermore, exemplary Ms temperatures range between approximately 10 degrees Celsius and approximately 20 degrees Celsius, and exemplary M_{f} temperatures range between approximately −1 degrees Celsius and approximately 15 degrees Celsius. The size of the annuloplasty ring can be changed all at once or incrementally in small steps at different times in order to achieve the adjustment necessary to produce the desired clinical result.

Certain shape memory alloys may further include a rhombohedral phase, having a rhombohedral start temperature (Rₛ) and a rhombohedral finish temperature (R_{f}), that exists between the austenite and martensite phases. An example of such a shape memory alloy is a NiTi alloy, which is commercially available from Memry Corporation (Bethel, Connecticut). In certain embodiments, an exemplary Rₛ temperature range is between approximately 30 degrees Celsius and approximately 50 degrees Celsius, and an exemplary R_{f} temperature range is between approximately 20 degrees Celsius and approximately 35 degrees Celsius. One benefit of using a shape memory material having a rhombohedral phase is that in the rhomobohedral phase the shape memory material may experience a partial physical distortion, as compared to the generally rigid structure of the austenite phase and the generally deformable structure of the martensite phase.

Certain shape memory alloys exhibit a ferromagnetic shape memory effect wherein the shape memory alloy transforms from the martensite phase to the austenite phase when exposed to an external magnetic field. The term “ferromagnetic” as used herein is a broad term and is used in its ordinary sense and includes, without limitation, any material that easily magnetizes, such as a material having atoms that orient their electron spins to conform to an external magnetic field. Ferromagnetic materials include permanent magnets, which can be magnetized through a variety of modes, and materials, such as metals, that are attracted to permanent magnets. Ferromagnetic materials also include electromagnetic materials that are capable of being activated by an electromagnetic transmitter, such as one located outside the heart 100. Furthermore, ferromagnetic materials may include one or more polymer-bonded magnets, wherein magnetic particles are bound within a polymer matrix, such as a biocompatible polymer. The magnetic materials can comprise isotropic and/or anisotropic materials, such as for example NdFeB (Neodynium Iron Boron), SmCo (Samarium Cobalt), ferrite and/or AlNiCo (Aluminum Nickel Cobalt) particles.

Thus, an annuloplasty ring comprising a ferromagnetic shape memory alloy can be implanted in a first configuration having a first shape and later changed to a second configuration having a second (e.g., memorized) shape without heating the shape memory material above the Aₛ temperature. Advantageously, nearby healthy tissue is not exposed to high temperatures that could damage the tissue. Further, since the ferromagnetic shape memory alloy does not need to be heated, the size of the annuloplasty ring can be adjusted more quickly and more uniformly than by heat activation.

Exemplary ferromagnetic shape memory alloys include Fe—C, Fe—Pd, Fe—Mn—Si, Co—Mn, Fe—Co—Ni—Ti, Ni—Mn—Ga, Ni₂MnGa, Co—Ni—Al, and the like. Certain of these shape memory materials may also change shape in response to changes in temperature. Thus, the shape of such materials can be adjusted by exposure to a magnetic field, by changing the temperature of the material, or both.

In certain embodiments, combinations of different shape memory materials are used. For example, annuloplasty rings according to certain embodiments comprise a combination of shape memory polymer and shape memory alloy (e.g., NiTi). In certain such embodiments, an annuloplasty ring comprises a shape memory polymer tube and a shape memory alloy (e.g., NiTi) disposed within the tube. Such embodiments are flexible and allow the size and shape of the shape memory to be further reduced without impacting fatigue properties. In addition, or in other embodiments, shape memory polymers are used with shape memory alloys to create a bi-directional (e.g., capable of expanding and contracting) annuloplasty ring. Bi-directional annuloplasty rings can be created with a wide variety of shape memory material combinations having different characteristics.

In the following description, reference is made to the accompanying drawings, which form a part hereof, and which show, by way of illustration, specific embodiments or processes in which the invention may be practiced. Where possible, the same reference numbers are used throughout the drawings to refer to the same or like components. In some instances, numerous specific details are set forth in order to provide a thorough understanding of the present disclosure. The present disclosure, however, may be practiced without the specific details or with certain alternative equivalent components and methods to those described herein. In other instances, well-known components and methods have not been described in detail so as not to unnecessarily obscure aspects of the present disclosure.

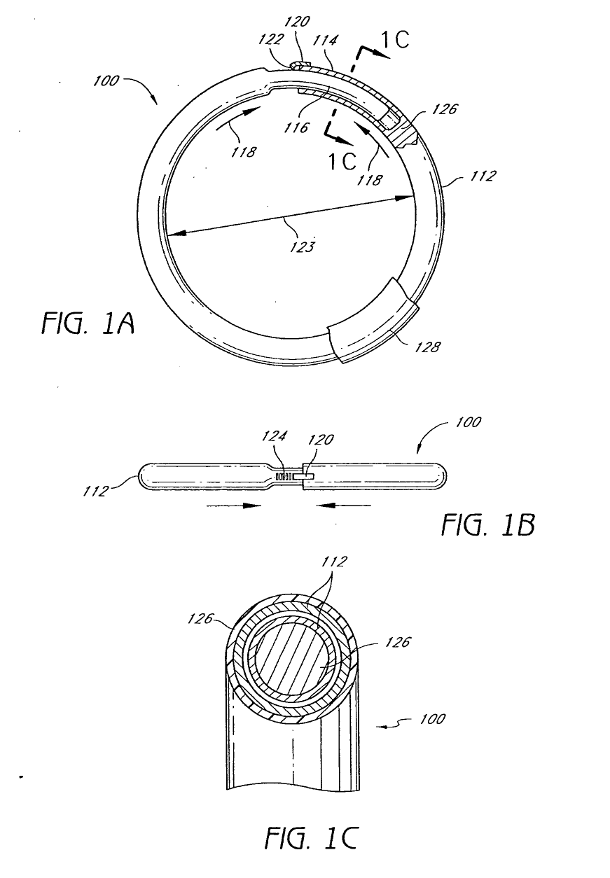

FIGS. 1A-1C illustrate an adjustable annuloplasty ring 100 according to certain embodiments that can be adjusted in vivo after implantation into a patient's body. The annuloplasty ring 100 has a substantially annular configuration and comprises a tubular body member 112 that folds back upon itself in a substantial circle having a nominal diameter as indicated by arrow 123. The tubular body member 112 comprises a receptacle end 114 and an insert end 116. The insert end 116 of the tubular member 112 is reduced in outer diameter or transverse dimension as compared to the receptacle end 114. As used herein, “dimension” is a broad term having its ordinary and customary meaning and includes a size or distance from a first point to a second point along a line or arc. For example, a dimension may be a circumference, diameter, radius, arc length, or the like. As another example, a dimension may be a distance between an anterior portion and a posterior portion of an annulus.

The receptacle end accepts the insert end 116 of the tubular member 112 to complete the ring-like structure of the annuloplasty ring 100. The insert end 116 slides freely within the receptacle end 114 of the annuloplasty ring 100 which allows contraction of the overall circumference of the ring 100 as the insert end 116 enters the receptacle end 114 as shown by arrows 118 in FIG. 1A. In certain embodiments, the nominal diameter or transverse dimension 123 of the annuloplasty ring 100 can be adjusted from approximately 25 mm to approximately 38 mm. However, an artisan will recognize from the disclosure herein that the diameter or transverse dimension 123 of the annuloplasty ring 100 can be adjusted to other sizes depending on the particular application. Indeed, the diameter or transverse dimension 123 of the annuloplasty ring 100 can be configured to reinforce body structures substantially smaller than 25 mm and substantially larger than 38 mm.

An artisan will recognize from the disclosure herein that in other embodiments the insert end 116 can couple with the receptacle end 114 without being inserted in the receptacle end 114. For example, the insert end 116 can overlap the receptacle end 114 such that it slides adjacent thereto. In other embodiments, for example, the ends 114, 116 may grooved to guide the movement of the adjacent ends 114, 116 relative to one another. Other embodiments within the scope of the invention will occur to those skilled in the art.

The annuloplasty ring 100 also comprises a suturable material 128, shown partially cut away in FIG. 1A, and not shown in FIGS. 1B and 1C for clarity. The suturable material 128 is disposed about the tubular member 112 to facilitate surgical implantation of the annuloplasty ring 100 in a body structure, such as about a heart valve annulus. In certain embodiments, the suturable material 128 comprises a suitable biocompatible material such as Dacron®, woven velour, polyurethane, polytetrafluoroethylene (PTFE), heparin-coated fabric, or the like. In other embodiments, the suturable material 128 comprises a biological material such as bovine or equine pericardium, homograft, patient graft, or cell-seeded tissue. The suturable material 128 may be disposed about the entire circumference of the tubular member 112, or selected portions thereof. For example, in certain embodiments, the suturable material 128 is disposed so as to enclose substantially the entire tubular member 112 except at the narrowed insert end 116 that slides into the receptacle end 118 of the tubular member 112.

As shown in FIGS. 1A and 1B, in certain embodiments, the annuloplasty ring 100 also comprises a ratchet member 120 secured to the receptacle end 114 of the tubular member 112. The ratchet member 120 comprises a pawl 122 configured to engage transverse slots 124 (shown in FIG. 1B) on the insert end 116 of the tubular member 112. The pawl 122 of the ratchet member 120 engages the slots 124 in such a way as to allow contraction of the circumference of the annuloplasty ring 100 and prevent or reduce circumferential expansion of the annuloplasty ring 100. Thus, the ratchet reduces unwanted circumferential expansion of the annuloplasty ring 100 after implantation due, for example, to dynamic forces on the annuloplasty ring 100 from the heart tissue during systolic contraction of the heart.

In certain embodiments, the tubular member 112 comprises a rigid material such as stainless steel, titanium, or the like, or a flexible material such as silicon rubber, Dacron®, or the like. In certain such embodiments, after implantation into a patient's body, the circumference of the annuloplasty ring 100 is adjusted in vivo by inserting a catheter (not shown) into the body and pulling a wire (not shown) attached to the tubular member 112 through the catheter to manually slide the insert end 116 of the tubular member 112 into the receptacle end 114 of the tubular member 112. As the insert end 116 slides into the receptacle end 114, the pawl 122 of the ratchet member 120 engages the slots 124 on the insert end 116 to hold the insert end 116 in the receptacle end 114. Thus, for example, as the size of a heart valve annulus reduces after implantation of the annuloplasty ring 100, the size of the annuloplasty ring 100 can also be reduced to provide an appropriate amount of reinforcement to the heart valve.

In certain other embodiments, the tubular member 112 comprises a shape memory material that is responsive to changes in temperature and/or exposure to a magnetic field. As discussed above, the shape memory material may include shape memory polymers (e.g., polylactic acid (PLA), polyglycolic acid (PGA)) and/or shape memory alloys (e.g., nickel-titanium) including ferromagnetic shape memory alloys (e.g., Fe—C, Fe—Pd, Fe—Mn—Si, Co—Mn, Fe—Co—Ni—Ti, Ni—Mn—Ga, Ni₂MnGa, Co—Ni—Al). In certain such embodiments, the annuloplasty ring 100 is adjusted in vivo by applying an energy source such as radio frequency energy, X-ray energy, microwave energy, ultrasonic energy such as high intensity focused ultrasound (HIFU) energy, light energy, electric field energy, magnetic field energy, combinations of the foregoing, or the like. Preferably, the energy source is applied in a non-invasive manner from outside the body. For example, as discussed above, a magnetic field and/or RF pulses can be applied to the annuloplasty ring 100 within a patient's body with an apparatus external to the patient's body such as is commonly used for magnetic resonance imaging (MRI). However, in other embodiments, the energy source may be applied surgically such as by inserting a catheter into the body and applying the energy through the catheter.

In certain embodiments, the tubular body member 112 comprises a shape memory material that responds to the application of temperature that differs from a nominal ambient temperature, such as the nominal body temperature of 37 degrees Celsius for humans. The tubular member 112 is configured to respond by starting to contract upon heating the tubular member 112 above the Aₛ temperature of the shape memory material. In certain such embodiments, the annuloplasty ring 100 has an initial diameter or transverse dimension 123 of approximately 30 mm, and contracts or shrinks to a transverse dimension 123 of approximately 23 mm to approximately 28 mm, or any increment between those values. This produces a contraction percentage in a range between approximately 6 percent and approximately 23 percent, where the percentage of contraction is defined as a ratio of the difference between the starting diameter and finish diameter divided by the starting diameter.

The activation temperatures (e.g., temperatures ranging from the As temperature to the A_{f} temperature) at which the tubular member 112 contracts to a reduced circumference may be selected and built into the annuloplasty ring 100 such that collateral damage is reduced or eliminated in tissue adjacent the annuloplasty ring 100 during the activation process. Exemplary A_{f} temperatures for the shape memory material of the tubular member 112 at which substantially maximum contraction occurs are in a range between approximately 38 degrees Celsius and approximately 76 degrees Celsius. In certain embodiments, the A_{f} temperature is in a range between approximately 39 degrees Celsius and approximately 75 degrees Celsius. For some embodiments that include shape memory polymers for the tubular member 112, activation temperatures at which the glass transition of the material or substantially maximum contraction occur range between approximately 38 degrees Celsius and approximately 60 degrees Celsius. In other such embodiments, the activation temperature is in a range between approximately 40 degrees Celsius and approximately 59 degrees Celsius.

In certain embodiments, the tubular member 112 is shape set in the austenite phase to a remembered configuration during the manufacturing of the tubular member 112 such that the remembered configuration is that of a relatively small circumferential value with the insert end 116 fully inserted into the receptacle end 114. After cooling the tubular member 112 below the M_{f} temperature, the tubular member 112 is manually deformed to a larger circumferential value with the insert end 116 only partially inserted into the receptacle end 114 to achieve a desired starting nominal circumference for the annuloplasty ring 100. In certain such embodiments, the tubular member 112 is sufficiently malleable in the martensite phase to allow a user such as a physician to adjust the circumferential value by hand to achieve a desired fit with the heart valve annulus. In certain embodiments, the starting nominal circumference for the annuloplasty ring 100 is configured to improve leaflet coaptation and reduce regurgitation in a heart valve.

After implantation, the annuloplasty ring 100 is preferably activated non-invasively by the application of energy to the patient's body to heat the tubular member 112. In certain embodiments, an MRI device is used as discussed above to heat the tubular member 112, which then causes the shape memory material of the tubular member 112 to transform to the austenite phase and remember its contracted configuration. Thus, the circumference of the annuloplasty ring 100 is reduced in vivo without the need for surgical intervention. Standard techniques for focusing the magnetic field from the MRI device onto the annuloplasty ring 100 may be used. For example, a conductive coil can be wrapped around the patient in an area corresponding to the annuloplasty ring 100. In other embodiments, the shape memory material is activated by exposing it other sources of energy, as discussed above.

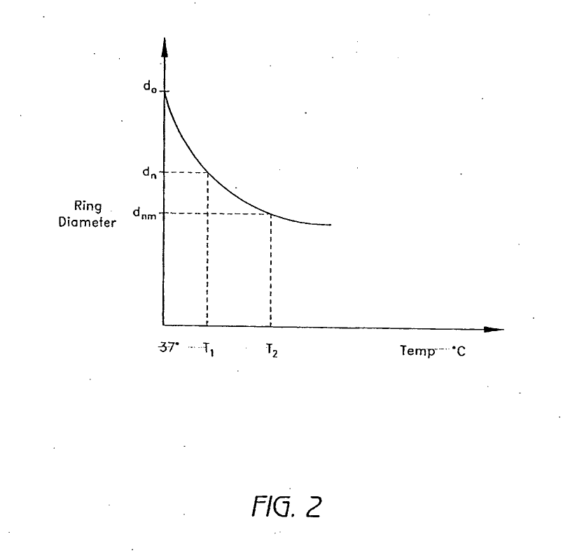

The circumference reduction process, either non-invasively or through a catheter, can be carried out all at once or incrementally in small steps at different times in order to achieve the adjustment necessary to produce the desired clinical result. If heating energy is applied such that the temperature of the tubular member 112 does not reach the A_{f} temperature for substantially maximum transition contraction, partial shape memory transformation and contraction may occur. FIG. 2 graphically illustrates the relationship between the temperature of the tubular member 112 and the diameter or transverse dimension 123 of the annuloplasty ring 100 according to certain embodiments. At body temperature of approximately 37 degrees Celsius, the diameter of the tubular member 112 has a first diameter d₀. The shape memory material is then increased to a first raised temperature T₁. In response, the diameter of the tubular member 112 reduces to a second diameter dₙ. The diameter of the tubular member 112 can then be reduced to a third diameter dₙₘ by raising the temperature to a second temperature T₂.

As graphically illustrated in FIG. 2, in certain embodiments, the change in diameter from do to dₙₘ is substantially continuous as the temperature is increased from body temperature to T₂. For example, in certain embodiments a magnetic field of about 2.5 Tesla to about 3.0 Tesla is used to raise the temperature of the tubular member 112 above the A_{f} temperature to complete the austenite phase and return the tubular member 112 to the remembered configuration with the insert end 116 fully inserted into the receptacle end 114. However, a lower magnetic field (e.g., 0.5 Tesla) can initially be applied and increased (e.g., in 0.5 Tesla increments) until the desired level of heating and desired contraction of the annuloplasty ring 100 is achieved. In other embodiments, the tubular member 112 comprises a plurality of shape memory materials with different activation temperatures and the diameter of the tubular member 112 is reduced in steps as the temperature increases.

Whether the shape change is continuous or stepped, the diameter or transverse dimension 123 of the ring 100 can be assessed or monitored during the contraction process to determine the amount of contraction by use of MRI imaging, ultrasound imaging, computed tomography (CT), X-ray or the like. If magnetic energy is being used to activate contraction of the ring 100, for example, MRI imaging techniques can be used that produce a field strength that is lower than that required for activation of the annuloplasty ring 100.

In certain embodiments, the tubular member 112 comprises an energy absorption enhancement material 126. As shown in FIGS. 1A and 1C, the energy absorption enhancement material 126 may be disposed within an inner chamber of the tubular member 112. As shown in FIG. 1C (and not shown in FIG. 1A for clarity), the energy absorption enhancement material 126 may also be coated on the outside of the tubular member 112 to enhance energy absorption by the tubular member 112. For embodiments that use energy absorption enhancement material 126 for enhanced absorption, it may be desirable for the energy absorption enhancement material 126, a carrier material (not shown) surrounding the energy absorption enhancement material 126, if there is one, or both to be thermally conductive. Thus, thermal energy from the energy absorption enhancement material 126 is efficiently transferred to the shape memory material of the annuloplasty ring 100, such as the tubular member 112.

As discussed above, the energy absorption enhancement material 126 may include a material or compound that selectively absorbs a desired heating energy and efficiently converts the non-invasive heating energy to heat which is then transferred by thermal conduction to the tubular member 112. The energy absorption enhancement material 126 allows the tubular member 112 to be actuated and adjusted by the non-invasive application of lower levels of energy and also allows for the use of non-conducting materials, such as shape memory polymers, for the tubular member 112. For some embodiments, magnetic flux ranging between about 2.5 Tesla and about 3.0 Tesla may be used for activation. By allowing the use of lower energy levels, the energy absorption enhancement material 126 also reduces thermal damage to nearby tissue. Suitable energy absorption enhancement materials 126 are discussed above.

In certain embodiments, a circumferential contraction cycle can be reversed to induce an expansion of the annuloplasty ring 100. Some shape memory alloys, such as NiTi or the like, respond to the application of a temperature below the nominal ambient temperature. After a circumferential contraction cycle has been performed, the tubular member 112 is cooled below the Mₛ temperature to start expanding the annuloplasty ring 100. The tubular member 112 can also be cooled below the M_{f} temperature to finish the transformation to the martensite phase and reverse the contraction cycle. As discussed above, certain polymers also exhibit a two-way shape memory effect and can be used to both expand and contract the annuloplasty ring 100 through heating and cooling processes. Cooling can be achieved, for example, by inserting a cool liquid onto or into the annuloplasty ring 100 through a catheter, or by cycling a cool liquid or gas through a catheter placed near the annuloplasty ring 100. Exemplary temperatures for a NiTi embodiment for cooling and reversing a contraction cycle range between approximately 20 degrees Celsius and approximately 30 degrees Celsius.

In certain embodiments, external stresses are applied to the tubular member 112 during cooling to expand the annuloplasty ring 100. In certain such embodiments, one or more biasing elements (not shown) are operatively coupled to the tubular member 112 so as to exert a circumferentially expanding force thereon. For example, in certain embodiments a biasing element such as a spring (not shown) is disposed in the receptacle end 114 of the tubular member 112 so as to push the insert end 16 at least partially out of the receptacle end 114 during cooling. In such embodiments, the tubular member 112 does not include the ratchet member 120 such that the insert end 116 can slide freely into or out of the receptacle end 114.

In certain embodiments, the tubular member comprises ferromagnetic shape memory material, as discussed above. In such embodiments, the diameter of the tubular member 112 can be changed by exposing the tubular member 112 to a magnetic field. Advantageously, nearby healthy tissue is not exposed to high temperatures that could damage the tissue. Further, since the shape memory material does not need to be heated, the size of the tubular member 112 can be adjusted more quickly and more uniformly than by heat activation.

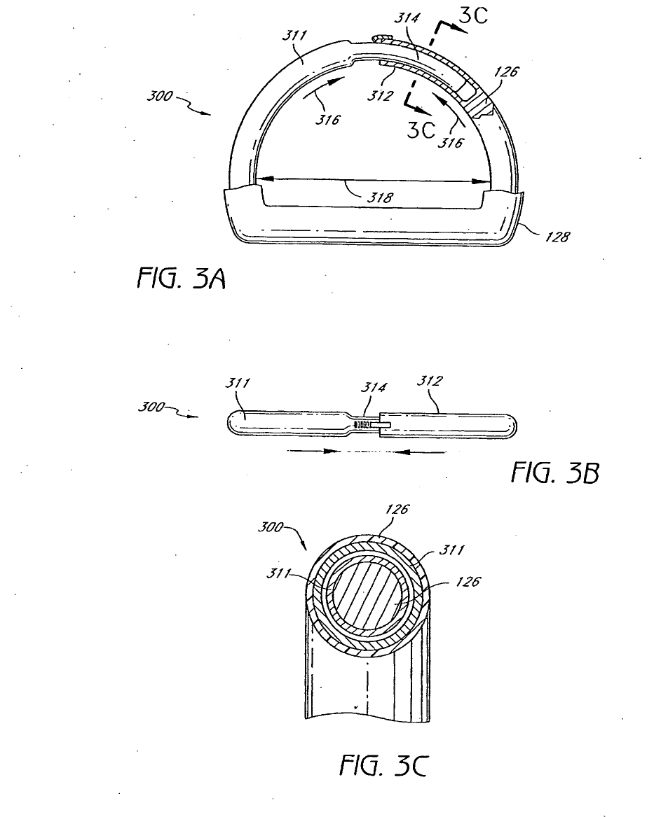

FIGS. 3A-3C illustrate an embodiment of an adjustable annuloplasty ring 300 that is similar to the annuloplasty ring 100 discussed above, but having a D-shaped configuration instead of a circular configuration. The annuloplasty ring 300 comprises a tubular body member 311 having a receptacle end 312 and an insert end 314 sized and configured to slide freely in the hollow receptacle end 312 in an axial direction which allows the annuloplasty ring 300 to constrict upon activation to a lesser circumference or transverse dimension as indicated by arrows 316. The annuloplasty ring 300 has a major transverse dimension indicated by arrow 318 that is reduced upon activation of the annuloplasty ring 300. The major transverse dimension indicated by arrow 318 can be the same as or similar to the transverse dimension indicated by arrow 123 discussed above. In certain embodiments, the features, dimensions and materials of the annuloplasty ring 300 are the same as or similar to the features, dimensions and materials of annuloplasty ring 100 discussed above. The D-shaped configuration of ring 32 allows a proper fit of the ring 32 with the morphology of some particular heart valves.

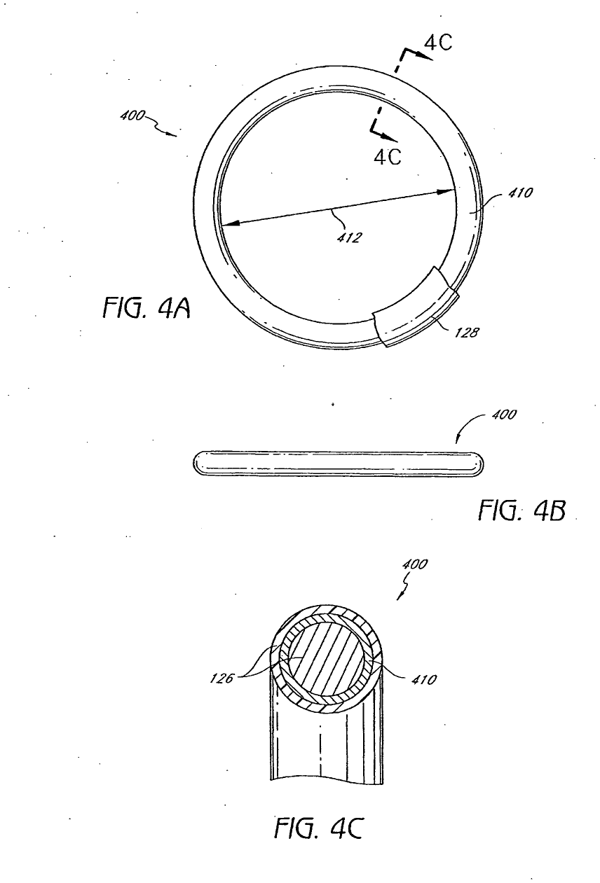

FIGS. 4A-4C show an embodiment of an annuloplasty ring 400 that includes a continuous tubular member 410 surrounded by a suturable material 128. The tubular member 410 has a substantially circular transverse cross section, as shown in FIG. 4C, and has an absorption enhancing material 126 disposed within an inner chamber of the tubular member 410. In certain embodiments, the absorption enhancing material 126 is also disposed on the outer surface of the tubular member 410. The tubular member 410 may be made from a shape memory material such as a shape memory polymer or a shape memory alloy including a ferromagnetic shape memory alloy, as discussed above.

For embodiments of the annuloplasty ring 400 with a tubular member 410 made from a continuous piece of shape memory alloy (e.g., NiTi alloy) or shape memory polymer, the annuloplasty ring 400 can be activated by the surgical and/or non-invasive application of heating energy by the methods discussed above with regard to other embodiments. For embodiments of the annuloplasty ring 400 with a tubular member 410 made from a continuous piece of ferromagnetic shape memory alloy, the annuloplasty ring 400 can be activated by the non-invasive application of a suitable magnetic field. The annuloplasty ring 400 has a nominal inner diameter or transverse dimension indicated by arrow 412 in FIG. 4A that is set during manufacture of the ring 400. In certain embodiments, the annuloplasty ring 400 is sufficiently malleable when it is implanted into a patient's body that it can be adjusted by hand to be fitted to a particular heart valve annulus.

In certain embodiments, upon activating the tubular member 410 by the application of energy, the tubular member 410 remembers and assumes a configuration wherein the transverse dimension is less than the nominal transverse dimension 412. A contraction in a range between approximately 6 percent to approximately 23 percent may be desirable in some embodiments which have continuous hoops of shape memory tubular members 410. In certain embodiments, the tubular member 410 comprises a shape memory NiTi alloy having an inner transverse dimension in a range between approximately 25 mm and approximately 38 mm. In certain such embodiments, the tubular member 410 can contract or shrink in a range between approximately 6 percent and approximately 23 percent, where the percentage of contraction is defined as a ratio of the difference between the starting diameter and finish diameter divided by the starting diameter. In certain embodiments, the annuloplasty ring 400 has a nominal inner transverse dimension 412 of approximately 30 mm and an inner transverse dimension in a range between approximately 23 mm and approximately 128 mm in a fully contracted state.

As discussed above in relation to FIG. 2, in certain embodiments, the inner transverse dimension 412 of certain embodiments can be altered as a function of the temperature of the tubular member 410. As also discussed above, in certain such embodiments, the progress of the size change can be measured or monitored in real-time conventional imaging techniques. Energy from conventional imaging devices can also be used to activate the shape memory material and change the inner transverse dimension 412 of the tubular member 410. In certain embodiments, the features, dimensions and materials of the annuloplasty ring 400 are the same as or similar to the features, dimensions and materials of the annuloplasty ring 100 discussed above. For example, in certain embodiments, the tubular member 410 comprises a shape memory material that exhibits a two-way shape memory effect when heated and cooled. Thus, the annuloplasty ring 400, in certain such embodiments, can be contracted and expanded.

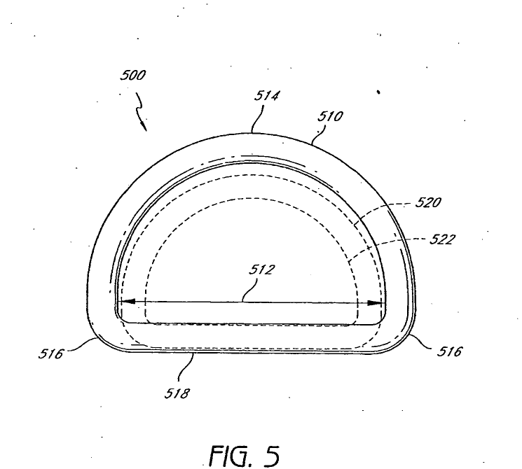

FIG. 5 illustrates a top view of an annuloplasty ring 500 having a D-shaped configuration according to certain embodiments. The annuloplasty ring 500 includes a continuous tubular member 510 comprising a shape memory material that has a nominal inner transverse dimension indicated by arrow 512 that may contract or shrink upon the activation of the shape memory material by surgically or non-invasive applying energy thereto, as discussed above. The tubular member 510 may comprise a homogeneous shape memory material, such as a shape memory polymer or a shape memory alloy including, for example, a ferromagnetic shape memory alloy.

Alternatively, the tubular member 510 may comprise two or more sections or zones of shape memory material having different temperature response curves. The shape memory response zones may be configured in order to achieve a desired configuration of the annuloplasty ring 500 as a whole when in a contracted state, either fully contracted or partially contracted. For example, the tubular member 510 may have a first zone or section 514 that includes the arched portion of the tubular member that terminates at or near the comers 516 and a second zone or section 518 that includes the substantially straight portion of the tubular member 510 disposed directly between the comers 516.

The annuloplasty ring 500 is shown in a contracted state in FIG. 5 as indicated by the dashed lines 520, 522, which represent contracted states of certain embodiments wherein both the first section 514 and second section 518 of the tubular member 510 have contracted axially. A suturable material (not shown), such as the suturable material 128 shown in FIG. 1, may be disposed about the tubular member 510 and the tubular member 510 may comprise or be coated with an energy absorption enhancement material 126, as discussed above. In certain embodiments, the features, dimensions and materials of the annuloplasty ring 500 are the same as or similar to the features, dimensions and materials of the annuloplasty ring 100 discussed above.

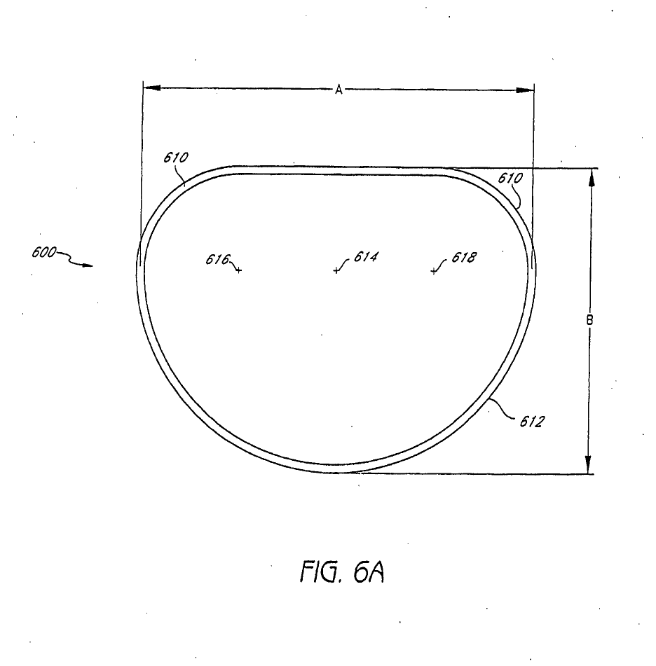

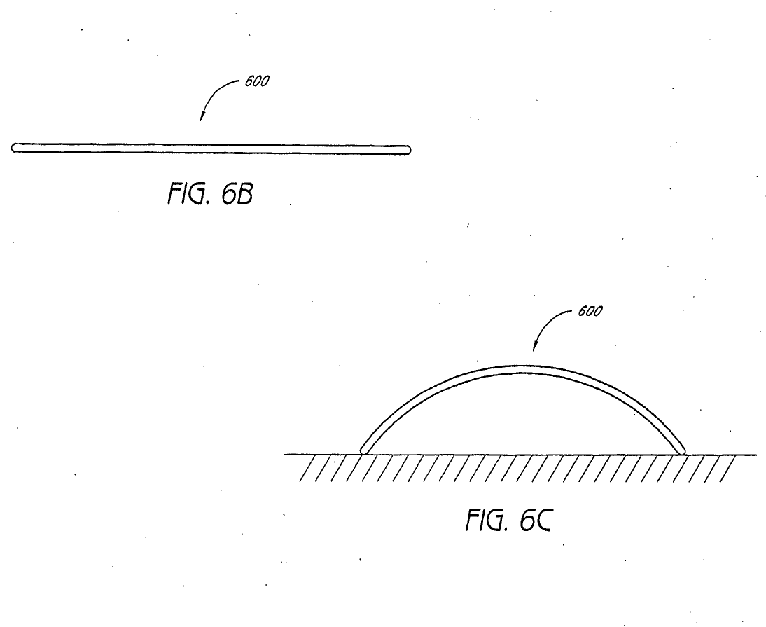

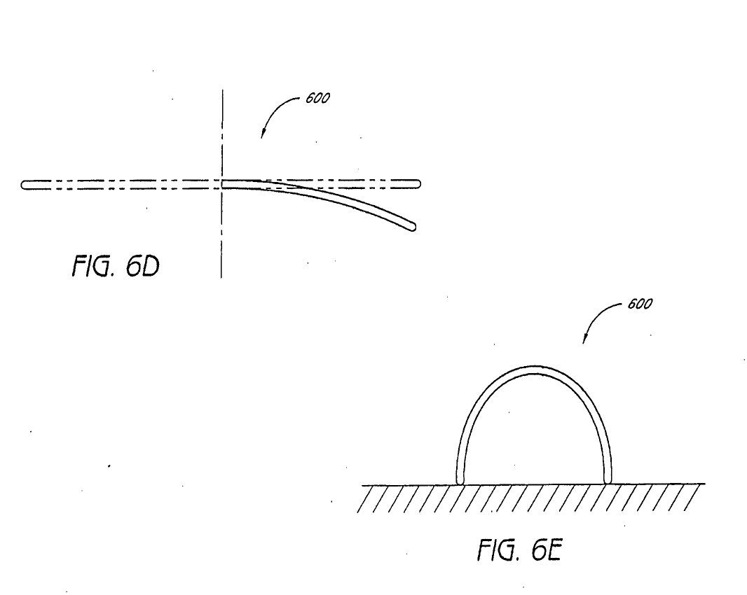

FIG. 6A is a schematic diagram of a top view of a substantially D-shaped wire 600 comprising a shape memory material according to certain embodiments of the invention. The term “wire” is a broad term having its normal and customary meaning and includes, for example, mesh, flat, round, rod-shaped, or band-shaped members. Suitable shape memory materials include shape memory polymers or shape memory alloys including, for example, ferromagnetic shape memory alloys, as discussed above. The wire 600 comprises a substantially linear portion 608, two corner portions 610, and a substantially semi-circular portion 612.

For purposes of discussion, the wire 600 is shown relative to a first reference point 614, a second reference point 616 and a third reference point 618. The radius of the substantially semi-circular portion 612 is defined with respect to the first reference point 614 and the corner portions 610 are respectively defined with respect to the second reference point 616 and the third reference point 618. Also for purposes of discussion, FIG. 6A shows a first transverse dimension A, a second transverse dimension B.

In certain embodiments, the first transverse dimension A is in a range between approximately 20.0 mm and approximately 40.0 mm, the second transverse dimension B is in a range between approximately 10.0 mm and approximately 25.0 mm. In certain such embodiments, the wire 600 comprises a rod having a diameter in a range between approximately 0.45 mm and approximately 0.55 mm, the radius of each corner portion 610 is in a range between approximately 5.8 mm and 7.2 mm, and the radius of the substantially semi-circular portion 612 is in a range between approximately 11.5 mm and approximately 14.0 mm. In certain other such embodiments, the wire 600 comprises a rod having a diameter in a range between approximately 0.90 mm and approximately 1.10 mm, the radius of each corner portion 610 is in a range between approximately 6.1 mm and 7.4 mm, and the radius of the substantially semi-circular portion 612 is in a range between approximately 11.7 mm and approximately 14.3 mm.

In certain other embodiments, the first transverse dimension A is in a range between approximately 26.1 mm and approximately 31.9 mm, the second transverse dimension B is in a range between approximately 20.3 mm and approximately 24.9 mm. In certain such embodiments, the wire 600 comprises a rod having a diameter in a range between approximately 0.4 mm and approximately 0.6 mm, the radius of each corner portion 610 is in a range between approximately 6.7 mm and 8.3 mm, and the radius of the substantially semi-circular portion 612 is in a range between approximately 13.3 mm and approximately 16.2 mm. In certain other such embodiments, the wire 600 comprises a rod having a diameter in a range between approximately 0.90 mm and approximately 1.10 mm, the radius of each corner portion 610 is in a range between approximately 6.9 mm and 8.5 mm, and the radius of the substantially semi-circular portion 612 is in a range between approximately 13.5 mm and approximately 16.5 mm.

In certain embodiments, the wire 600 comprises a NiTi alloy configured to transition to its austenite phase when heated so as to transform to a memorized shape, as discussed above. In certain such embodiments, the first transverse dimension A of the wire 600 is configured to be reduced by approximately 10% to 25% when transitioning to the austenite phase. In certain such embodiments, the austenite start temperature As is in a range between approximately 33 degrees Celsius and approximately 43 degrees Celsius, the austenite finish temperature A_{f} is in a range between approximately 45 degrees Celsius and approximately 55 degrees Celsius, the martensite start temperature Mₛ is less than approximately 30 degrees Celsius, and the martensite finish temperature M_{f} is greater than approximately 20 degrees Celsius. In other embodiments, the austenite finish temperature A_{f} is in a range between approximately 48.75 degrees Celsius and approximately 51.25 degrees Celsius. Other embodiments can include other start and finish temperatures for martensite, rhombohedral and austenite phases as described herein.

FIGS. 6B-6E are schematic diagrams of side views of the shape memory wire 600 of FIG. 6A according to certain embodiments. In addition to expanding and/or contracting the first transverse dimension A and/or the second transverse dimension B when transitioning to the austenite phase, in certain embodiments the shape memory wire 600 is configured to change shape in a third dimension perpendicular to the first transverse dimension A and the second transverse dimension B. For example, in certain embodiments, the shape memory wire 600 is substantially planar or flat in the third dimension, as shown in FIG. 6B, when implanted into a patient's body. Then, after implantation, the shape memory wire 600 is activated such that it expands or contracts in the first transverse dimension A and/or the second transverse dimension B and flexes or bows in the third dimension such that it is no longer planar, as shown in FIG. 6C. Such bowing may be symmetrical as shown in FIG. 6C or asymmetrical as shown in FIG. 6D to accommodate the natural shape of the annulus.

In certain embodiments, the shape memory wire 600 is configured to bow in the third dimension a distance in a range between approximately 2 millimeters and approximately 10 millimeters. In certain embodiments, the shape memory wire 600 is implanted so as to bow towards the atrium when implanted around a cardiac valve annulus to accommodate the natural shape of the annulus. In other embodiments, the shape memory wire 600 is configured to bow towards the ventricle when implanted around a cardiac valve to accommodate the natural shape of the annulus.

In certain embodiments, the shape memory wire 600 is bowed in the third dimension, as shown in FIG. 6C, when implanted into the patient's body. Then, after implantation, the shape memory wire 600 is activated such that it expands or contracts in the first transverse dimension A and/or the second transverse dimension B and further flexes or bows in the third dimension, as shown in FIG. 6E. In certain other embodiments, the shape memory wire 600 is bowed in the third dimension, as shown in FIG. 6C, when implanted into the patient's body. Then, after implantation, the shape memory wire 600 is activated such that it expands or contracts in the first transverse dimension A and/or the second transverse dimension B and changes shape in the third dimension so as to become substantially flat, as shown in FIG. 6B. An artisan will recognize from the disclosure herein that other annuloplasty rings disclosed herein can also be configured to bow or change shape in a third dimension so as to accommodate or further reinforce a valve annulus.

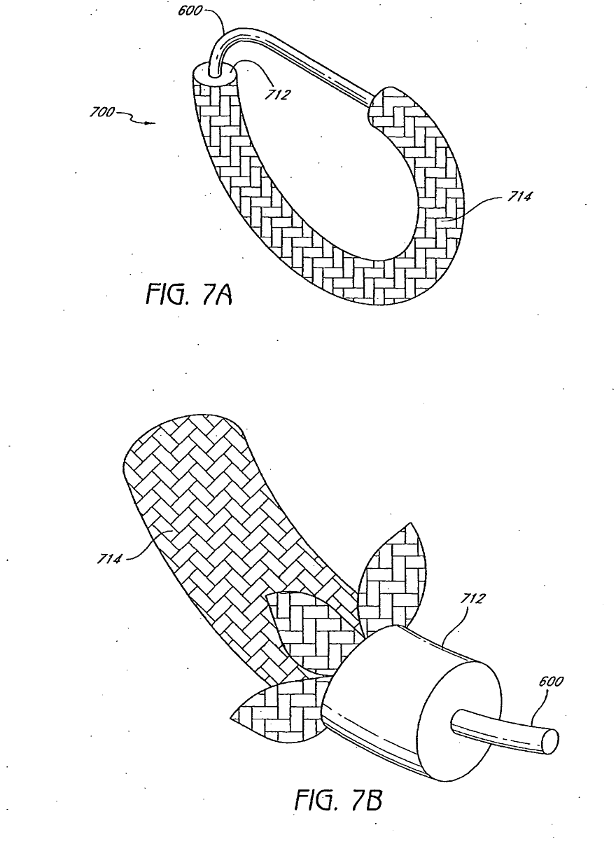

FIG. 7A is a perspective view illustrating portions of an annuloplasty ring 700 comprising the wire 600 shown in FIG. 6A according to certain embodiments of the invention. The wire 600 is covered by a flexible material 712 such as silicone rubber and a suturable material 714 such as woven polyester cloth, Dacron®, woven velour, polyurethane, polytetrafluoroethylene (PTFE), heparin-coated fabric, or other biocompatible material. In other embodiments, the suturable material 714 comprises a biological material such as bovine or equine pericardium, homograft, patient graft, or cell-seeded tissue. For illustrative purposes, portions of the flexible material 712 and the suturable material 714 are not shown in FIG. 7A to show the wire 600. However, in certain embodiments, the flexible material 712 and the suturable material 714 are continuous and cover substantially the entire wire 600. Although not shown, in certain embodiments, the wire 600 is coated with an energy absorption enhancement material, as discussed above.

FIG. 7B is an enlarged perspective view of a portion of the annuloplasty ring 700 shown in FIG. 7A. For illustrative purposes, portions of the flexible material 712 are not shown to expose the wire 600 and portions of the suturable material 714 are shown peeled back to expose the flexible material 712. In certain embodiments, the diameter of the flexible material 712 is in a range between approximately 0.10 inches and approximately 0.15 inches. FIG. 7B shows the wire 600 substantially centered within the circumference of the flexible material 712. However, in certain embodiments, the wire 600 is offset within the circumference of the flexible material 712 to allow more space for sutures.

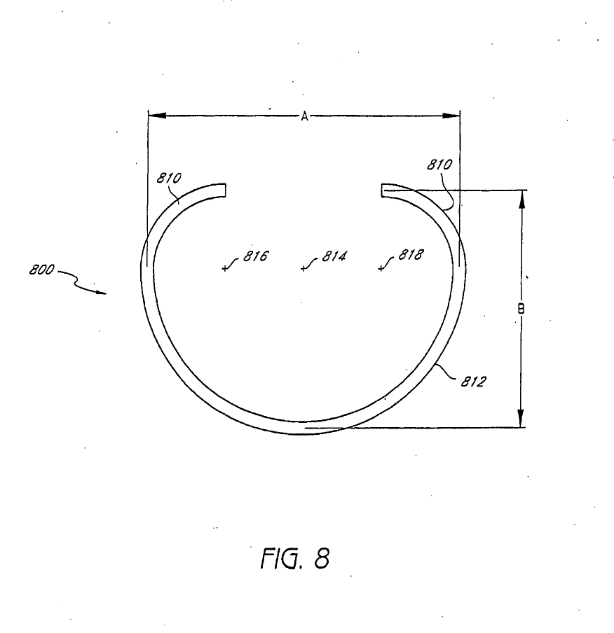

FIG. 8 is a schematic diagram of a substantially C-shaped wire 800 comprising a shape memory material according to certain embodiments of the invention. Suitable shape memory materials include shape memory polymers or shape memory alloys including, for example, ferromagnetic shape memory alloys, as discussed above. The wire 800 comprises two corner portions 810, and a substantially semi-circular portion 812.

For purposes of discussion, the wire 800 is shown relative to a first reference point 814, a second reference point 816 and a third reference point 818. The radius of the substantially semi-circular portion 812 is defined with respect to the first reference point 814 and the corner portions 810 are respectively defined with respect to the second reference point 816 and the third reference point 818. Also for purposes of discussion, FIG. 8 shows a first transverse dimension A and a second transverse dimension B. In certain embodiments, the wire 800 comprises a rod having a diameter and dimensions A and B as discussed above in relation to FIG. 6A.

In certain embodiments, the wire 800 comprises a NiTi alloy configured to transition to its austenite phase when heated so as to transform to a memorized shape, as discussed above. In certain such embodiments, the first transverse dimension A of the wire 800 is configured to be reduced by approximately 10% to 25% when transitioning to the austenite phase. In certain such embodiments, the austenite start temperature Aₛ is in a range between approximately 33 degrees Celsius and approximately 43 degrees Celsius, the austenite finish temperature A_{f} is in a range between approximately 45 degrees Celsius and approximately 55 degrees Celsius, the martensite start temperature Mₛ is less than approximately 30 degrees Celsius, and the martensite finish temperature M_{f} is greater than approximately 20 degrees Celsius. In other embodiments, the austenite finish temperature A_{f} is in a range between approximately 48.75 degrees Celsius and approximately 51.25 degrees Celsius.

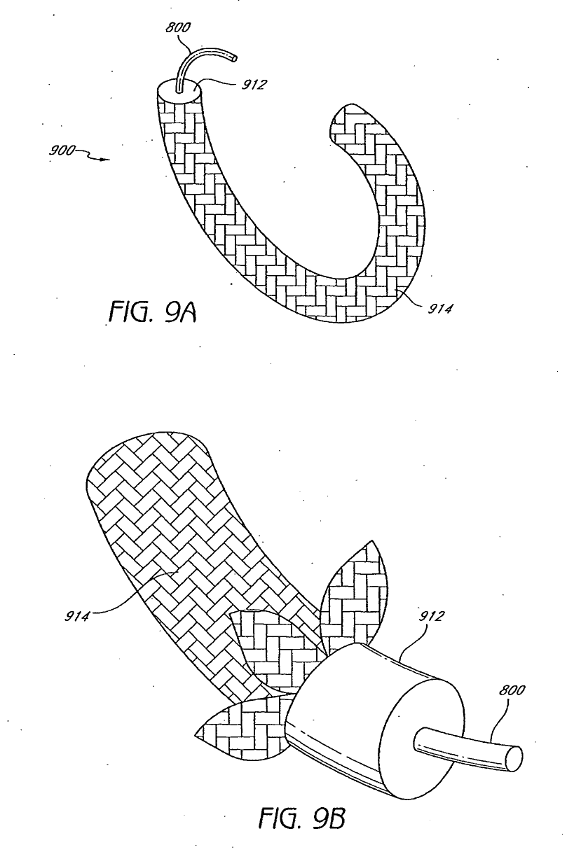

FIG. 9A is a perspective view illustrating portions of an annuloplasty ring 900 comprising the wire 800 shown in FIG. 8 according to certain embodiments of the invention. The wire 800 is covered by a flexible material 912 such as silicone rubber and a suturable material 914 such as woven polyester cloth, Dacron®, woven velour, polyurethane, polytetrafluoroethylene (PTFE), heparin-coated fabric, or other biocompatible material. In other embodiments, the suturable material 914 comprises a biological material such as bovine or equine pericardium, homograft, patient graft, or cell-seeded tissue. For illustrative purposes, portions of the flexible material 912 and the suturable material 914 are not shown in FIG. 9A to show the wire 800. However, in certain embodiments, the flexible material 912 and the suturable material 914 cover substantially the entire wire 800. Although not shown, in certain embodiments, the wire 800 is coated with an energy absorption enhancement material, as discussed above.

FIG. 9B is an enlarged perspective view of a portion of the annuloplasty ring 900 shown in FIG. 9A. For illustrative purposes, portions of the flexible material 912 are not shown to expose the wire 800 and portions of the suturable material 914 are shown peeled back to expose the flexible material 912. In certain embodiments, the diameter of the flexible material 912 is in a range between approximately 0.10 inches and approximately 0.15 inches. FIG. 9B shows the wire 800 substantially centered within the circumference of the flexible material 912. However, in certain embodiments, the wire 800 is offset within the circumference of the flexible material 912 to allow more space for sutures.

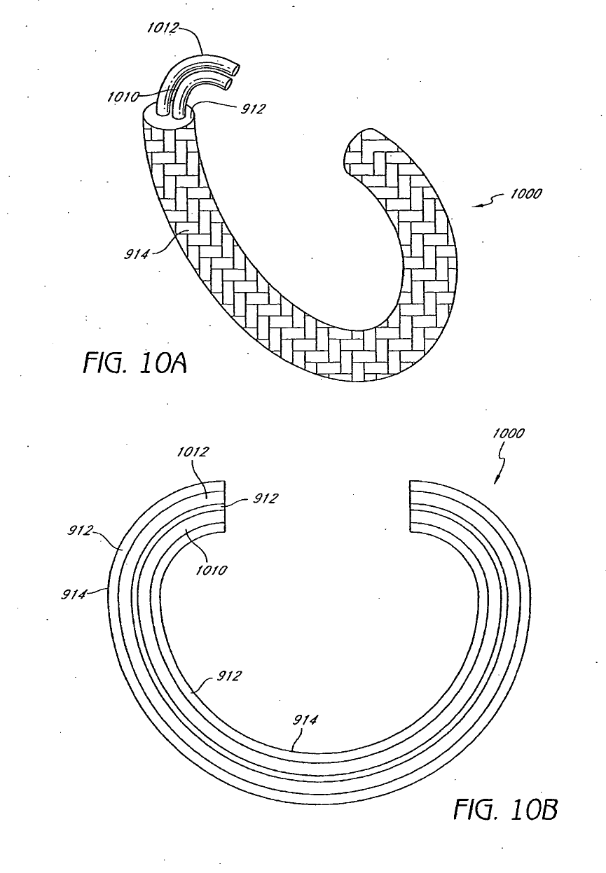

FIG. 10A is a perspective view illustrating portions of an annuloplasty ring 1000 configured to contract and expand according to certain embodiments of the invention. FIG. 10B is a top cross-sectional view of the annuloplasty ring 1000. As discussed above, after the annuloplasty ring 1000 has been contracted, it may become necessary to expand the annuloplasty ring 1000. For example, the annuloplasty ring 1000 may be implanted in a child with an enlarged heart. When the size of the heart begins to recover to its natural size, the annuloplasty ring 1000 can be contracted. Then, as the child gets older and the heart begins to grow, the annuloplasty ring 1000 can be enlarged as needed.

The annuloplasty ring 1000 comprises a first shape memory wire 1010 for contracting the annuloplasty ring 1000 and a second shape memory wire 1012 for expanding the annuloplasty ring 1000. The first and second shape memory wires, 1010, 1012 are covered by the flexible material 912 and the suturable material 914 shown in FIGS. 9A-9B. For illustrative purposes, portions of the flexible material 912 and the suturable material 914 are not shown in FIG. 10A to show the shape memory wires 1010, 1012. However, as schematically illustrated in FIG. 10B, in certain embodiments, the flexible material 912 and the suturable material 914 substantially cover the first and second shape memory wires 1010, 1012. As discussed below, the flexible material 912 operatively couples the first shape memory wire 1010 and the second shape memory wire 1012 such that a shape change in one will mechanically effect the shape of the other. The first and second shape memory wires 1010, 1012 each comprise a shape memory material, such as the shape memory materials discussed above. However, the first and second shape memory wires 1010, 1012 are activated at different temperatures.

In certain embodiments, the annuloplasty ring 1000 is heated to a first temperature that causes the first shape memory wire 1010 to transition to its austenite phase and contract to its memorized shape. At the first temperature, the second shape memory wire 1012 is in its martensite phase and is substantially flexible as compared the contracted first shape memory wire 1010. Thus, when the first shape memory wire 1010 transitions to its austenite phase, it exerts a sufficient force on the second shape memory wire 1012 through the flexible material 912 to deform the second shape memory wire 1012 and cause the annuloplasty ring 1000 to contract.

The annuloplasty ring 1000 can be expanded by heating the annuloplasty ring to a second temperature that causes the second shape memory wire 1012 to transition to its austenite phase and expand to its memorized shape. In certain embodiments, the second temperature is higher than the first temperature. Thus, at the second temperature, both the first and second shape memory wires 1010, 1012 are in their respective austenite phases. In certain such embodiments, the diameter of the second shape memory wire 1012 is sufficiently larger than the diameter of the first shape memory wire 1010 such that the second memory shape wire 1012 exerts a greater force to maintain its memorized shape in the austenite phase than the first shape memory wire 1010. Thus, the first shape memory wire 1010 is mechanically deformed by the force of the second memory shape wire 1012 and the annuloplasty ring 1000 expands.

In certain embodiments, the first memory shape wire 1010 is configured to contract by approximately 10% to 25% when transitioning to its austenite phase. In certain such embodiments, the first memory shape wire 1010 has an austenite start temperature Aₛ in a range between approximately 33 degrees Celsius and approximately 43 degrees Celsius, an austenite finish temperature A_{f} in a range between approximately 45 degrees Celsius and approximately 55 degrees Celsius, a martensite start temperature Mₛ less than approximately 30 degrees Celsius, and a martensite finish temperature M_{f} greater than approximately 20 degrees Celsius. In other embodiments, the austenite finish temperature A_{f} of the first memory shape wire 1010 is in a range between approximately 48.75 degrees Celsius and approximately 51.25 degrees Celsius.

In certain embodiments, the second memory shape wire 1012 is configured to expand by approximately 10% to 25% when transitioning to its austenite phase. In certain such embodiments, the second memory shape wire 1010 has an austenite start temperature As in a range between approximately 60 degrees Celsius and approximately 70 degrees Celsius, an austenite finish temperature A_{f} in a range between approximately 65 degrees Celsius and approximately 75 degrees Celsius, a martensite start temperature Mₛ less than approximately 30 degrees Celsius, and a martensite finish temperature M_{f} greater than approximately 20 degrees Celsius. In other embodiments, the austenite finish temperature A_{f} of the first memory shape wire 1010 is in a range between approximately 68.75 degrees Celsius and approximately 71.25 degrees Celsius.

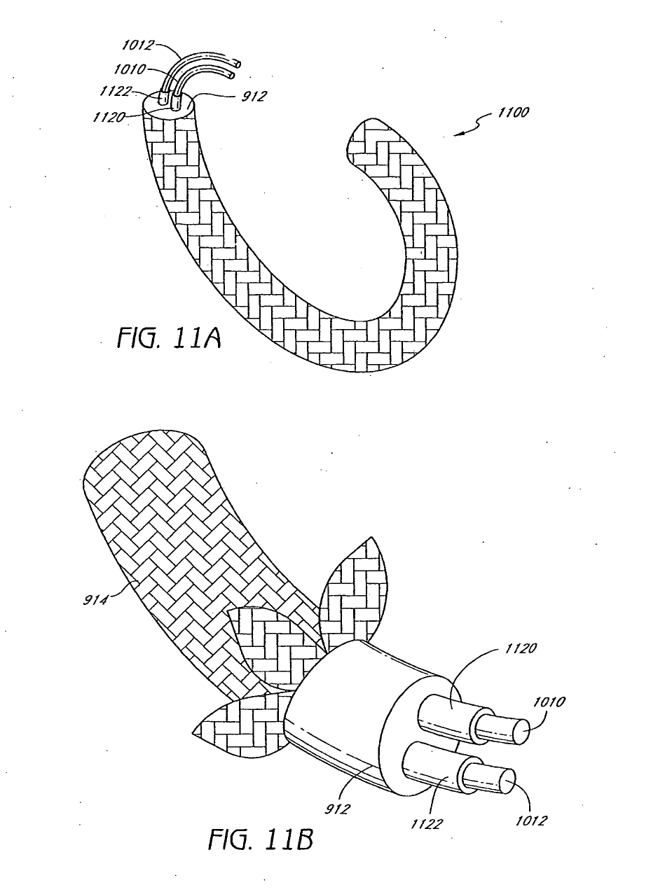

FIG. 11A is a perspective view illustrating portions of an annuloplasty ring 1100 according to certain embodiments comprising the first shape memory wire 1010 for contraction, the second shape memory wire 1012 for expansion, the flexible material 912 and the suturable material 914 shown in FIGS. 10A-10B. For illustrative purposes, portions of the flexible material 912 and the suturable material 914 are not shown in FIG. 11A to show the shape memory wires 1010, 1012. However, in certain embodiments, the flexible material 912 and the suturable material 914 substantially cover the first and second shape memory wires 1010, 1012. FIG. 11B is an enlarged perspective view of a portion of the annuloplasty ring 1100 shown in FIG. 11A. For illustrative purposes, portions of the flexible material 912 are not shown to expose the first and second shape memory wires 1010, 1012 and portions of the suturable material 914 are shown peeled back to expose the flexible material 912.

The first shape memory wire 1010 comprises a first coating 1120 and the second shape memory wire 1012 comprises a second coating 1122. In certain embodiments, the first coating 1120 and the second coating 1122 each comprise silicone tubing configured to provide suture attachment to a heart valve annulus. In certain other embodiments, the first coating 1120 and the second coating 1122 each comprise an energy absorption material, such as the energy absorption materials discussed above. In certain such embodiments, the first coating 1120 heats when exposed to a first form of energy and the second coating 1122 heats when exposed to a second form of energy. For example, the first coating 1120 may heat when exposed to MRI energy and the second coating 1122 may heat when exposed to HIFU energy. As another example, the first coating 1120 may heat when exposed to RF energy at a first frequency and the second coating 1122 may heat when exposed to RF energy at a second frequency. Thus, the first shape memory wire 1010 and the second shape memory wire 1012 can be activated independently such that one transitions to its austenite phase while the other remains in its martensite phase, resulting in contraction or expansion of the annuloplasty ring 1100.

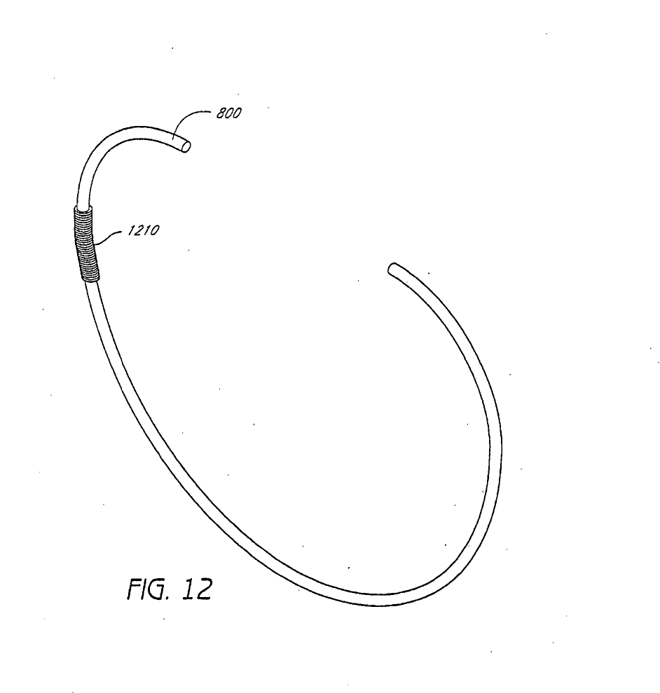

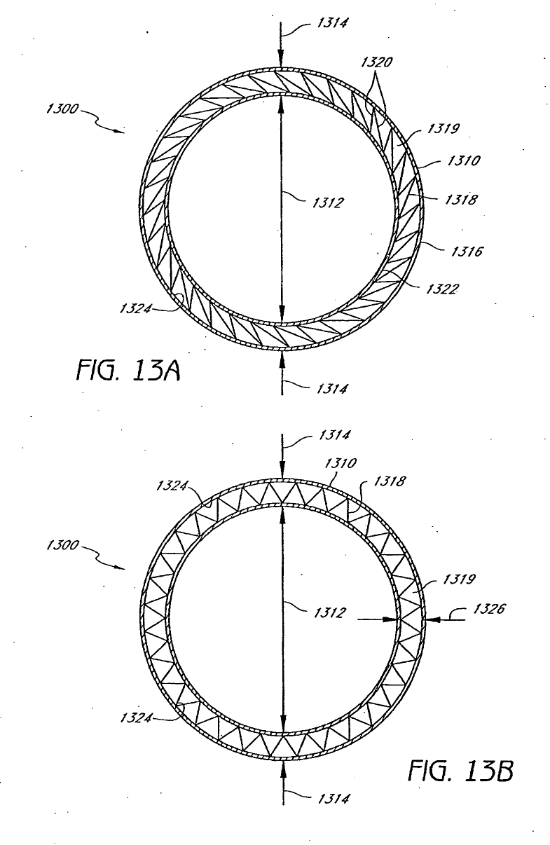

FIG. 12 is a perspective view of a shape memory wire 800, such as the wire 800 shown in FIG. 8, wrapped in an electrically conductive coil 1210 according to certain embodiments of the invention. The coil 1210 is wrapped around a portion of the wire 800 where it is desired to focus energy and heat the wire 800. In certain embodiments, the coil 1210 is wrapped around approximately 5% to approximately 15% of the wire 800. In other embodiments, the coil 1210 is wrapped around approximately 15% to approximately 70% of the wire 800. In other embodiments, the coil 1210 is wrapped around substantially the entire wire 800. Although not shown, in certain embodiments, the wire 800 also comprises a coating comprising an energy absorption material, such as the energy absorption materials discussed above. The coating may or may not be covered by the coil 1210.

As discussed above, an electrical current can be non-invasively induced in the coil 1210 using electromagnetic energy. For example, in certain embodiments, a handheld or portable device (not shown) comprising an electrically conductive coil generates an electromagnetic field that non-invasively penetrates the patient's body and induces a current in the coil 1210. The electrical current causes the coil 1210 to heat. The coil 1210, the wire 800 and the coating (if any) are thermally conductive so as to transfer the heat or thermal energy from the coil 1210 to the wire 800. Thus, thermal energy can be directed to the wire 800, or portions thereof, while reducing thermal damage to surrounding tissue.

FIGS. 13A and 13B show an embodiment of an annuloplasty ring 1310 having a nominal inner diameter or transverse dimension indicated by arrow 1312 and a nominal outer diameter or transverse dimension indicated by arrows 1314. The ring 1310 includes a tubular member 1316 having a substantially round transverse cross section with an internal shape memory member 1318 disposed within an inner chamber 1319 of the tubular member 1316. The internal shape memory member 1318 is a ribbon or wire bent into a series of interconnected segments 1320. Upon heating of the tubular member 1316 and the internal shape memory member 1318, the inner transverse dimension 1312 becomes smaller due to axial shortening of the tubular member 1316 and an inward radial force applied to an inner chamber surface 1322 of the tubular member 1316 by the internal shape memory member 1318. The internal shape memory member 1318 is expanded upon heating such that the ends of segments 1320 push against the inner chamber surface 1322 and outer chamber surface 1324, as shown by arrow 1326 in FIG. 13B, and facilitate radial contraction of the inner transverse dimension 1312. Thus, activation of the internal shape memory member 1318 changes the relative distance between the against the inner chamber surface 1322 and outer chamber surface 1324.

Although not shown in FIGS. 13A or 13B, The inner shape memory member 1318 may also have a heating energy absorption enhancement material, such as one or more of the energy absorption enhancement materials discussed above, disposed about it within the inner chamber 1319. The energy absorption material may also be coated on an outer surface and/or an inner surface of the tubular member 1316. The inner transverse dimension 1312 of the ring 1310 in FIG. 13B is less than the inner transverse dimension 1312 of the ring 1310 shown in FIG. 13A. However, according to certain embodiments, the outer transverse dimension 1314 is substantially constant in both FIGS. 13A and 13B.

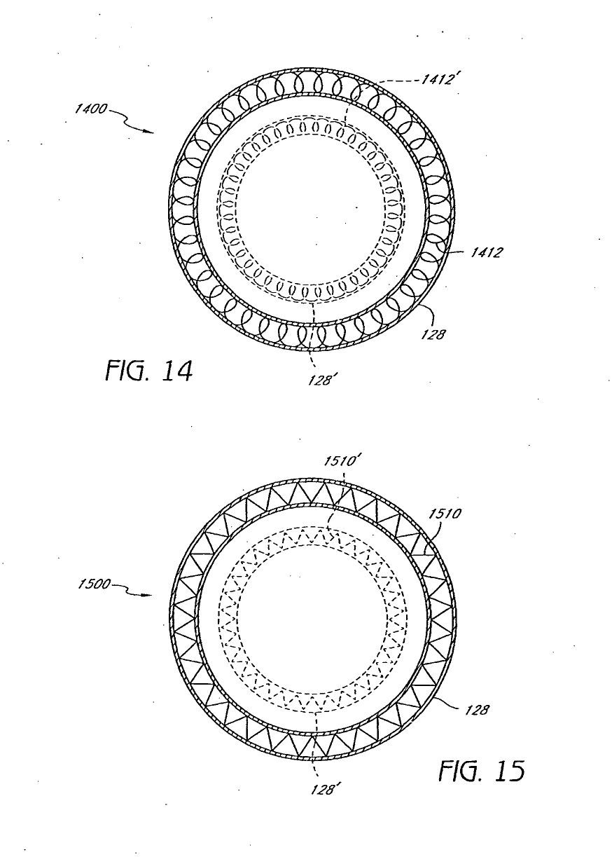

For some indications, it may be desirable for an adjustable annuloplasty ring to have some compliance in order to allow for expansion and contraction of the ring in concert with the expansion and contraction of the heart during the beating cycle or with the hydrodynamics of the pulsatile flow through the valve during the cycle. As such, it may be desirable for an entire annuloplasty ring, or a section or sections thereof, to have some axial flexibility to allow for some limited and controlled expansion and contraction under clinical conditions. FIGS. 14 and 15 illustrate embodiments of adjustable annuloplasty rings that allow some expansion and contraction in a deployed state.

FIG. 14 shows an annuloplasty ring 1400 that is constructed in such a way that it allows mechanical expansion and compression of the ring 1400 under clinical conditions. The ring 1400 includes a coil 1412 made of a shape memory material, such as one or more of the shape memory materials discussed above. The shape memory material or other portion of the ring 1400 may be coated with an energy absorption material, such as the energy absorption materials discussed above. The coil 1412 may have a typical helical structure of a normal spring wire coil, or alternatively, may have another structure such as a ribbon coil. In certain embodiments, the coil 1412 is surrounded by a suturable material 128, such as Dacron® or the other suturable materials discussed herein. The coiled structure or configuration of the coil 1412 allows the ring 1400 to expand and contract slightly when under physiological pressures and forces from heart dynamics or hydrodynamics of blood flow through a host heart valve.

For embodiments where the coil 1412 is made of NiTi alloy or other shape memory material, the ring 1400 is responsive to temperature changes which may be induced by the application of heating energy on the coil 1412. In certain embodiments, if the temperature is raised, the coil 1412 will contract axially or circumferentially such that an inner transverse dimension of the ring 1400 decreases, as shown by the dashed lines in FIG. 14. In FIG. 14, reference 1412′ represents the coil 1412 in its contracted state and reference 128′ represents the suturable material 128 in its contracted state around the contracted coil 1412′. In addition, or in other embodiments, the coil 1412 expands axially or circumferentially such that the inner transverse dimension of the ring 1400 increases. Thus, in certain embodiments, the ring 1400 can be expanded and contracted by applying invasive or non-invasive energy thereto.

FIG. 15 illustrates another embodiment of an adjustable annuloplasty ring 1500 that has dynamic compliance with dimensions, features and materials that may be the same as or similar to those of ring 1400. However, the ring 1500 has a zig-zag ribbon member 1510 in place of the coil 1412 in the embodiment of FIG. 14. In certain embodiments, if the temperature is raised, the ribbon member 1510 will contract axially or circumferentially such that an inner transverse dimension of the ring 1500 decreases, as shown by the dashed lines in FIG. 15. In FIG. 15, reference 1510′ represents the ribbon member 1510 in its contracted state and reference 128′ represents the suturable material 128 in its contracted state around the contracted ribbon member 1510′. In addition, or in other embodiments, the ribbon member 1510 expands axially or circumferentially such that the inner transverse dimension of the ring 1500 increases. Thus, in certain embodiments, the ring 1500 can be expanded and contracted by applying invasive or non-invasive energy thereto.

The embodiments of FIGS. 14 and 15 may have a substantially circular configuration as shown in the figures, or may have D-shaped or C-shaped configurations as shown with regard to other embodiments discussed above. In certain embodiments, the features, dimensions and materials of rings 1400 and 1500 are the same as or similar to the features, dimensions and materials of the annuloplasty ring 400 discussed above.

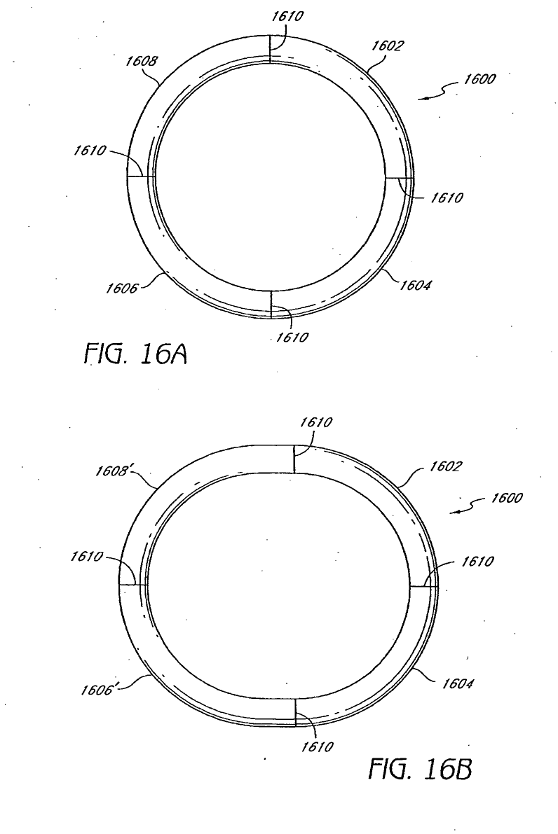

FIGS. 16A and 16B illustrate an annuloplasty ring 1600 according to certain embodiments that has a substantially circular shape or configuration when in the non-activated state shown in FIG. 16A. The ring 1600 comprises shape memory material or materials which are separated into a first temperature response zone 1602, a second temperature response zone 1604, a third temperature response zone 1606 and a fourth temperature response zone 1608. The zones are axially separated by boundaries 1610. Although the ring 1600 is shown with four zones 1602, 1604, 1606, 1608, an artisan will recognize from the disclosure herein that other embodiments may include two or more zones of the same or differing lengths. For example, one embodiment of an annuloplasty ring 1600 includes approximately three to approximately eight temperature response zones.

In certain embodiments, the shape memory materials of the various temperature response zones 1602, 1604, 1606, 1608 are selected to have temperature responses and reaction characteristics such that a desired shape and configuration can be achieved in vivo by the application of invasive or non-invasive energy, as discussed above. In addition to general contraction and expansion changes, more subtle changes in shape and configuration for improvement or optimization of valve function or hemodynamics may be achieved with such embodiments.

According to certain embodiments, the first zone 1602 and second zone 1604 of the ring 1600 are made from a shape memory material having a first shape memory temperature response. The third zone 1606 and fourth zone 1608 are made from a shape memory material having a second shape memory temperature response. In certain embodiments, the four zones comprise the same shape memory material, such as NiTi alloy or other shape memory material as discussed above, processed to produce the varied temperature response in the respective zones. In other embodiments, two or more of the zones may comprise different shape memory materials. Certain embodiments include a combination of shape memory alloys and shape memory polymers in order to achieve the desired results.

According to certain embodiments, FIG. 16B shows the ring 1600 after heat activation such that it comprises expanded zones 1606′, 1608′ corresponding to the zones 1606, 1608 shown in FIG. 16A. As schematically shown in FIG. 16A, activation has expanded the zones 1606′, 1608′ so as to increase the axial lengths of the segments of the ring 1600 corresponding to those zones. In addition, or in other embodiments, the zones 1606 and 1608 are configured to contract by a similar percentage instead of expand. In other embodiments, the zones 1602, 1604, 1606, 1608 are configured to each have a different shape memory temperature response such that each segment corresponding to each zone 1602, 1604, 1606, 1608 could be activated sequentially.

FIG. 16B schematically illustrates that the zones 1606′, 1608′ have expanded axially (i.e., from their initial configuration as shown by the zones 1606, 1608 in FIG. 16A). In certain embodiments, the zones 1602, 1604 are configured to be thermally activated to remember a shape memory dimension or size upon reaching a temperature in a range between approximately 51 degrees Celsius and approximately 60 degrees Celsius. In certain such embodiments, the zones 1606 and 1608 are configured to respond at temperatures in a range between approximately 41 degrees Celsius and approximately 48 degrees Celsius. Thus, for example, by applying invasive or non-invasive energy, as discussed above, to the ring 1600 until the ring 1600 reaches a temperature of approximately 41 degrees Celsius to approximately 48 degrees Celsius, the zones 1606, 1608 will respond by expanding or contracting by virtue of the shape memory mechanism, and the zones 1602, 1604 will not.

In certain other embodiments, the zones 1602, 1604 are configured to expand or contract by virtue of the shape memory mechanism at a temperature in a range between approximately 50 degrees Celsius and approximately 60 degrees Celsius. In certain such embodiments, the zones 1606, 1608 are configured to respond at a temperature in a range between approximately 39 degrees Celsius and approximately 45 degrees Celsius.

In certain embodiments, the materials, dimensions and features of the annuloplasty ring 1600 and the corresponding zones 1602, 1604, 1606, 1608 have the same or similar features, dimensions or materials as those of the other ring embodiments discussed above. In certain embodiments, the features of the annuloplasty ring 1600 are added to the embodiments discussed above.

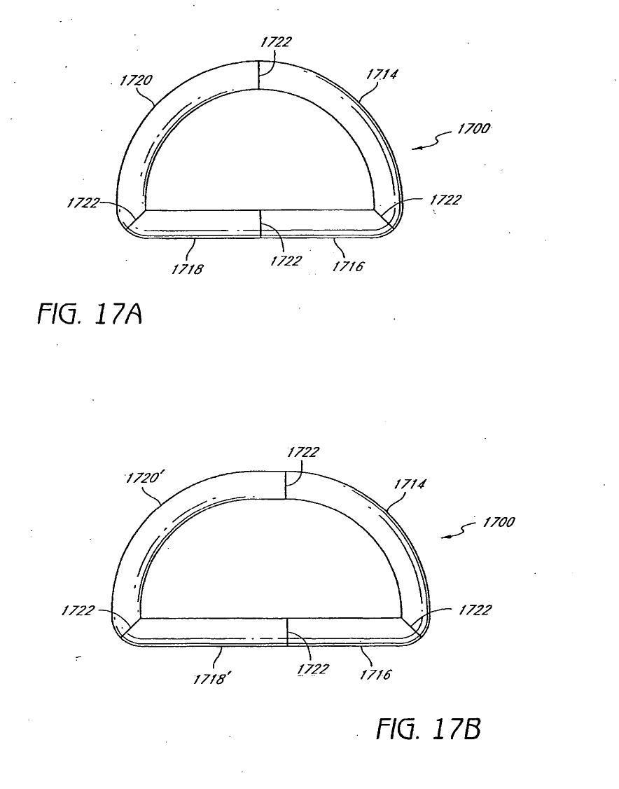

FIGS. 17A and 17B illustrate an annuloplasty ring 1700 according to certain embodiments that is similar to the annuloplasty ring 1600 discussed above, but having a “D-shaped” configuration. The ring 1700 comprises shape memory material or materials which are separated into a first temperature response zone 1714, a second temperature response zone 1716, a third temperature response zone 1718 and a fourth temperature response zone 1720. The segments defined by the zones 1714, 1716, 1718, 1720 are separated by boundaries 1722. Other than the D-shaped configuration, the ring 1700 according to certain embodiments has the same or similar features, dimensions and materials as the features, dimension and materials of the ring 1600 discussed above.

According to certain embodiments, FIG. 17B shows the ring 1700 after heat activation such that it comprises expanded zones 1718′, 1720′ corresponding to the zones 1718, 1720 shown in FIG. 17A. As schematically shown in FIG. 17B, activation has expanded the zones 1718′, 1720′ by virtue of the shape memory mechanism. The zones 1718, 1720 could also be selectively shrunk or contracted axially by virtue of the same shape memory mechanism for an embodiment having a remembered shape smaller than the nominal shape shown in FIG. 17A. The transverse cross sections of the rings 1600 and 1700 are substantially round, but can also have any other suitable transverse cross sectional configuration, such as oval, square, rectangular or the like.

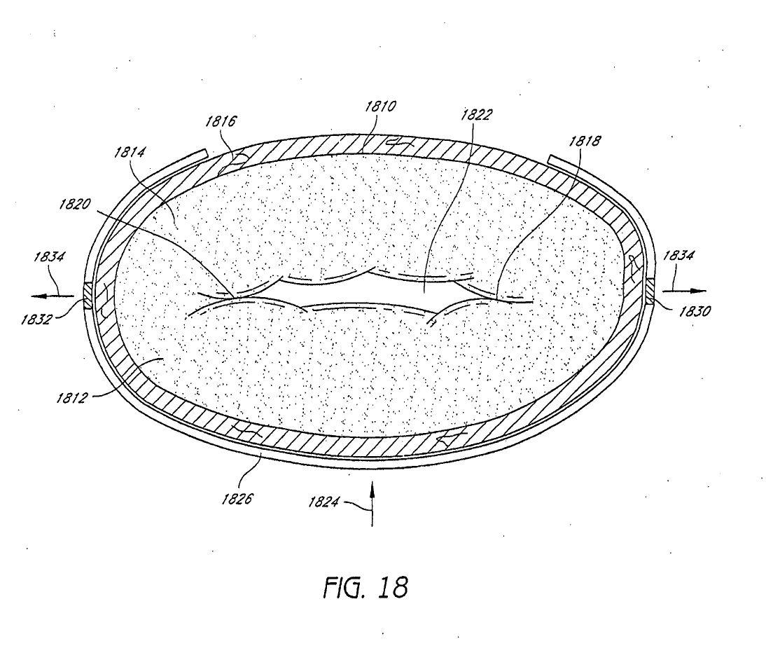

In certain situations, it is advantageous to reshape a heart valve annulus in one dimension while leaving another dimension substantially unchanged or reshaped in a different direction. For example, FIG. 18 is a sectional view of a mitral valve 1810 having an anterior (aortic) leaflet 1812, a posterior leaflet 1814 and an annulus 1816. The anterior leaflet 1812 and the posterior leaflet 1814 meet at a first commissure 1818 and a second commissure 1820. When healthy, the annulus 1816 encircles the leaflets 1812, 1814 and maintains their spacing to provide closure of a gap 1822 during left ventricular contraction. When the heart is not healthy, the leaflets 1812, 1814 do not achieve sufficient coaptation to close the gap 1822, resulting in regurgitation. In certain embodiments, the annulus 1816 is reinforced so as to push the anterior leaflet 1812 and the posterior leaflet 1814 closer together without substantially pushing the first commissure 1818 and the second commissure 1820 toward one another.

FIG. 18 schematically illustrates an exemplary annuloplasty ring 1826 comprising shape memory material configured to reinforce the annulus 1816 according to certain embodiments of the invention. For illustrative purposes, the annuloplasty ring 1826 is shown in an activated state wherein it has transformed to a memorized configuration upon application of invasive or non-invasive energy, as described herein. While the annuloplasty ring 1826 is substantially C-shaped, an artisan will recognize from the disclosure herein that other shapes are possible including, for example, a continuous circular, oval or D-shaped ring.

In certain embodiments, the annuloplasty ring 1826 comprises a first marker 1830 and a second marker 1832 that are aligned with the first commissure 1818 and the second commissure 1820, respectively, when the annuloplasty ring 1826 is implanted around the mitral valve 1810. In certain embodiments, the first marker 1830 and the second marker 1832 comprise materials that can be imaged in-vivo using standard imaging techniques. For example, in certain embodiments, the markers 1830 comprise radiopaque markers or other imaging materials, as is known in the art. Thus, the markers 1830, 1832 can be used for subsequent procedures for alignment with the annuloplasty ring 1826 and/or the commissures 1818, 1820. For example, the markers 1830, 1832 can be used to align a percutaneous energy source, such as a heated balloon inserted through a catheter, with the annuloplasty ring 1826.

When the shape memory material is activated, the annuloplasty ring 1826 contracts in the direction of the arrow 1824 to push the anterior leaflet 1812 toward the posterior leaflet 1814. Such anterior/posterior contraction improves the coaptation of the leaflets 1812, 1814 such that the gap 1824 between the leaflets 1812, 1814 sufficiently closes during left ventricular contraction. In certain embodiments, the annuloplasty ring 1826 also expands in the direction of arrows 1834. Thus, the first commissure 1818 and the second commissure 1820 are pulled away from each other, which draws the leaflets 1812, 1814 closer together and further improves their coaptation. However, in certain other embodiments, the annuloplasty ring does not expand in the direction of the arrows 1834. In certain such embodiments, the distance between the lateral portions of the annuloplasty ring 1826 between the anterior portion and the posterior portion (e.g., the lateral portions approximately correspond to the locations of the markers 1830, 1832 in the embodiment shown in FIG. 18) remains substantially the same after the shape memory material is activated.

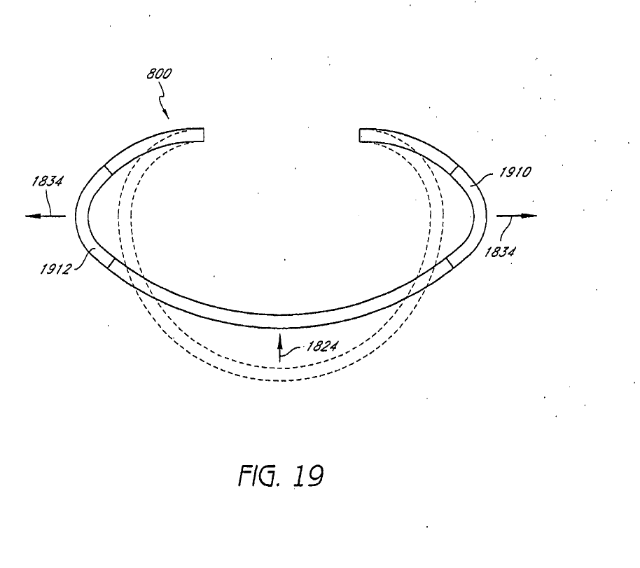

FIG. 19 is a schematic diagram of a substantially C-shaped wire comprising a shape memory material configured to contract in a first direction and expand in a second direction according to certain embodiments of the invention. Suitable shape memory materials include shape memory polymers or shape memory alloys including, for example, ferromagnetic shape memory alloys, as discussed above. FIG. 19 schematically illustrates the wire 800 in its activated configuration or memorized shape. For illustrative purposes, the wire 800 is shown relative to dashed lines representing its deformed shape or configuration when implanted into a body before activation.

When the shape memory material is activated, the wire 800 is configured to respond by contracting in a first direction as indicated by arrow 1824. In certain embodiments, the wire 800 also expands in a second direction as indicated by arrows 1834. Thus, the wire 800 is usable by the annuloplasty ring 1826 shown in FIG. 18 to improve the coaptation of the leaflets 1812, 1814 by contracting the annulus 1816 in the anterior/posterior direction. In certain embodiments, the anterior/posterior contraction is in a range between approximately 10% and approximately 20%. In certain embodiments, only a first portion 1910 and a second portion 1912 of the wire 800 comprise the shape memory material. When the shape memory material is activated, the first portion 1910 and the second portion 1912 of the wire 800 are configured to respond by transforming to their memorized configurations and reshaping the wire 800 as shown.

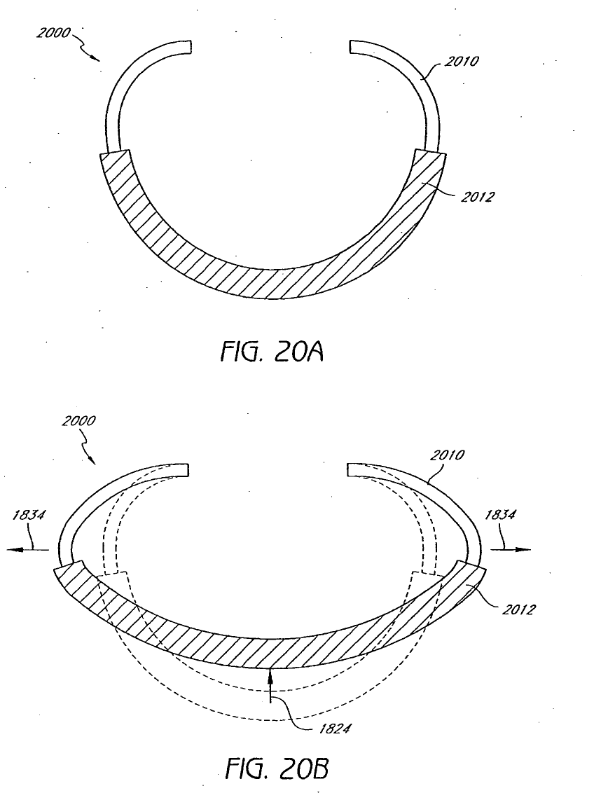

FIGS. 20A and 20B are schematic diagrams of a body member 2000 according to certain embodiments usable by an annuloplasty ring, such as the annuloplasty ring 1826 shown in FIG. 18. Although not shown, in certain embodiments, the body member 2000 is covered by a flexible material such as silicone rubber and a suturable material such as woven polyester cloth, Dacron®, woven velour, polyurethane, polytetrafluoroethylene (PTFE), heparin-coated fabric, or other biocompatible material, as discussed above.

The body member 2000 comprises a wire 2010 and a shape memory tube 2012. As used herein, the terms “tube,” “tubular member” and “tubular structure” are broad terms having at least their ordinary and customary meaning and include, for example, hollow elongated structures that may in cross-section be cylindrical, elliptical, polygonal, or any other shape. Further, the hollow portion of the elongated structure may be filled with one or more materials that may be the same as and/or different than the material of the elongated structure. In certain embodiments, the wire 2010 comprises a metal or metal alloy such as stainless steel, titanium, platinum, combinations of the foregoing, or the like. In certain embodiments, the shape memory tube 2012 comprises shape memory materials formed in a tubular structure through which the wire 2010 is inserted. In certain other embodiments, the shape memory tube 2012 comprises a shape memory material coated over the wire 2010. Suitable shape memory materials include shape memory polymers or shape memory alloys including, for example, ferromagnetic shape memory alloys, as discussed above. Although not shown, in certain embodiments, the body member 2000 comprises an energy absorption enhancement material, as discussed above.

FIG. 20A schematically illustrates the body member 2000 in a first configuration or shape and FIG. 20B schematically illustrates the body member 2000 in a second configuration or shape after the shape memory tube has been activated. For illustrative purposes, dashed lines in FIG. 20B also show the first configuration of the body member 2000. When the shape memory material is activated, the shape memory tube 2012 is configured to respond by contracting in a first direction as indicated by arrow 1824. In certain embodiments, the shape memory tube 2012 is also configured to expand in a second direction as indicated by arrows 1834. The transformation of the shape memory tube 2012 exerts a force on the wire 2010 so as to change its shape. Thus, the body member 2000 is usable by the annuloplasty ring 1826 shown in FIG. 18 to pull the commissures 1818, 1820 further apart and push the leaflets 1812, 1814 closer together to improve coaptation.

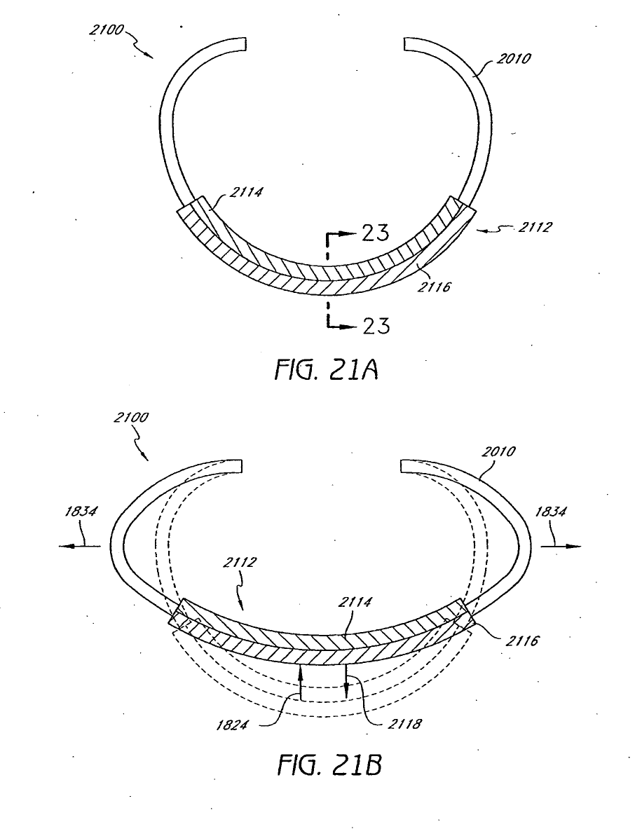

FIGS. 21A and 21B are schematic diagrams of a body member 2100 according to certain embodiments usable by an annuloplasty ring, such as the annuloplasty ring 1826 shown in FIG. 18. Although not shown, in certain embodiments, the body member 2100 is covered by a flexible material such as silicone rubber and a suturable material such as woven polyester cloth, Dacron®, woven velour, polyurethane, polytetrafluoroethylene (PTFE), heparin-coated fabric, or other biocompatible material, as discussed above.

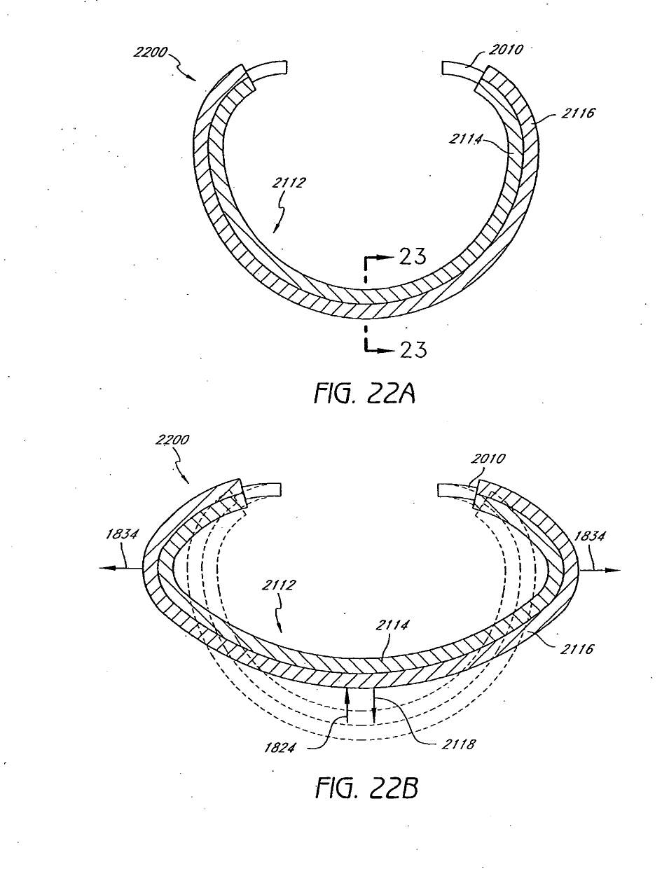

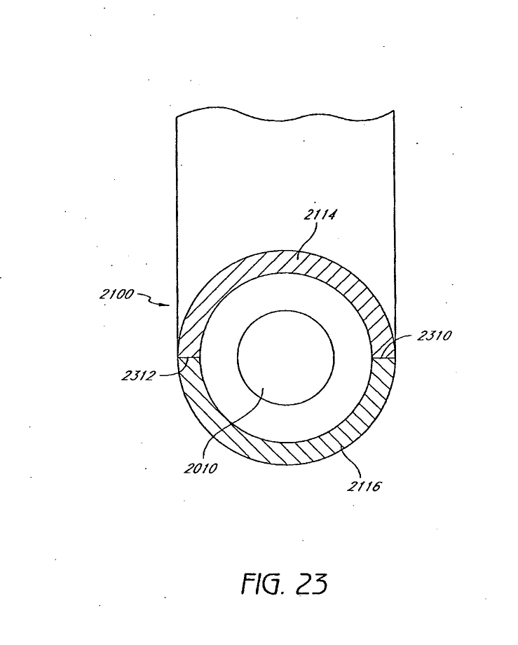

The body member 2100 comprises a wire 2010, such as the wire 2010 shown in FIGS. 20A and 20B and a shape memory tube 2112. As schematically illustrated in FIGS. 21A and 21B, the shape memory tube 2112 is sized and configured to cover a certain percentage of the wire 2010. However, an artisan will recognize from the disclosure herein that in other embodiments the shape memory tube 2112 may cover other percentages of the wire 2010. Indeed, FIGS. 22A and 22B schematically illustrate another embodiment of a body member 2200 comprising a shape memory tube 2112 covering a substantial portion of a wire 2010. The amount of coverage depends on such factors as the particular application, the desired shape change, the shape memory materials used, the amount of force to be exerted by the shape memory tube 2112 when changing shape, combinations of the foregoing, and the like. For example, in certain embodiments where, as in FIGS. 22A and 22B, the shape memory tube 2112 covers a substantial portion of a wire 2010, portions of the shape memory tube 2112 are selectively heated to reshape the wire 2010 at a particular location. In certain such embodiments, HIFU energy is directed towards, for example, the left side of the shape memory tube 2112, the right side of the shape memory tube 2112, the bottom side of the shape memory tube 2112, or a combination of the foregoing to activate only a portion of the shape memory tube 2112. Thus, the body member 2200 can be reshaped one or more portions at a time to allow selective adjustments.

In certain embodiments, the shape memory tube 2112 comprises a first shape memory material 2114 and a second shape memory material 2116 formed in a tubular structure through which the wire 2010 is inserted. In certain such embodiments, the first shape memory material 2114 and the second shape memory material 2116 are each configured as a semi-circular portion of the tubular structure. For example, FIG. 23 is a transverse cross-sectional view of the body member 2100. As schematically illustrated in FIG. 23, the first shape memory material 2114 and the second shape memory material 2116 are joined at a first boundary 2310 and a second boundary 2312. In certain embodiments, silicone tubing (not shown) holds the first shape memory material 2114 and the second shape memory material 2116 together. In certain other embodiments, the first shape memory material 2114 and the second shape memory material 2116 each comprise a shape memory coating covering opposite sides of the wire 2010. Suitable shape memory materials include shape memory polymers or shape memory alloys including, for example, ferromagnetic shape memory alloys, as discussed above. Although not shown, in certain embodiments the body member 2100 comprises an energy absorption enhancement material, as discussed above.

FIG. 21A schematically illustrates the body member 2100 in a first configuration or shape before the first shape memory material 2114 and the second shape memory material 2116 are activated. In certain embodiments, the first shape memory material 2114 and the second shape memory material 2116 are configured to be activated or return to their respective memorized shapes at different temperatures. Thus, the first shape memory material 2114 and the second shape memory material 2116 can be activated at different times to selectively expand and/or contract the body member 2100. For example, in certain embodiments, the second shape memory material 2116 is configured to be activated at a lower temperature than the first shape memory material 2114.

FIG. 21B schematically illustrates the body member 2100 in a second configuration or shape after the second shape memory material 2116 has been activated. For illustrative purposes, dashed lines in FIG. 21B also show the first configuration of the body member 2100. When the second shape memory material 2116 is activated, it responds by bending the body member 2100 in a first direction as indicated by arrow 1824. In certain embodiments, activation also expands the body member 2100 in a second direction as indicated by arrows 1834. Thus, the body member 2100 is usable by the annuloplasty ring 1826 shown in FIG. 18 to pull the commissures 1818, 1820 further apart and push the leaflets 1812, 1814 closer together to improve coaptation.

In certain embodiments, the first shape memory material 2114 can then be activated to bend the body member 2100 opposite to the first direction as indicated by arrow 2118. In certain such embodiments, the body member 2100 is reshaped to the first configuration as shown in FIG. 21A (or the dashed lines in FIG. 21B). Thus, for example, if the size of the patient's heart begins to grow again (e.g., due to age or illness), the body member 2100 can be enlarged to accommodate the growth. In certain other embodiments, activation of the first shape memory material 2114 further contracts the body member 2100 in the direction of the arrow 1824. In certain embodiments, the first shape memory material 2114 has an austenite start temperature Aₛ in a range between approximately 42 degrees Celsius and approximately 50 degrees Celsius and the second shape memory material 2116 has an austenite start temperature Aₛ in a range between approximately 38 degrees Celsius and 41 degrees Celsius.

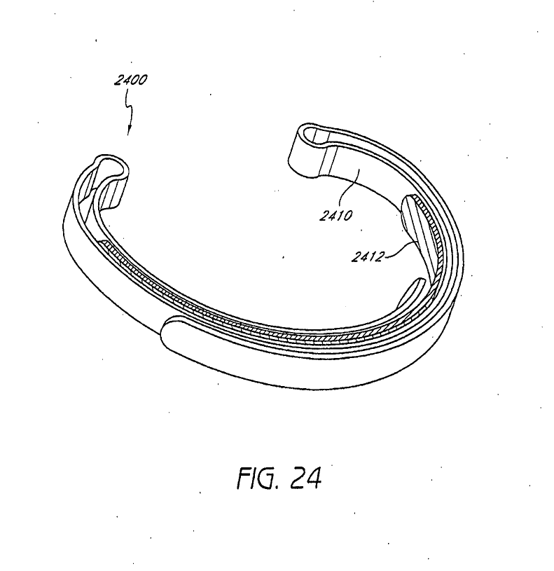

FIG. 24 is a perspective view of a body member 2400 usable by an annuloplasty ring according to certain embodiments comprising a first shape memory band 2410 and a second shape memory band 2412. Suitable shape memory materials for the bands 2410, 2412 include shape memory polymers or shape memory alloys including, for example, ferromagnetic shape memory alloys, as discussed above. Although not shown, in certain embodiments the body member 2100 comprises an energy absorption enhancement material, as discussed above. Although not shown, in certain embodiments, the body member 2100 is covered by a flexible material such as silicone rubber and a suturable material such as woven polyester cloth, Dacron®, woven velour, polyurethane, polytetrafluoroethylene (PTFE), heparin-coated fabric, or other biocompatible material, as discussed above.

The first shape memory band 2410 is configured to loop back on itself to form a substantially C-shaped configuration. However, an artisan will recognize from the disclosure herein that the first shape memory band 2410 can be configured to loop back on itself in other configurations including, for example, circular, D-shaped, or other curvilinear configurations. When activated, the first shape memory band 2410 expands or contracts such that overlapping portions of the band 2410 slide with respect to one another, changing the overall shape of the body member 2400. The second shape memory band 2412 is disposed along a surface of the first shape memory band 2410 such that the second shape memory band 2412 is physically deformed when the first shape memory band 2410 is activated, and the first shape memory band 2410 is physically deformed when the second shape memory band 2412 is activated.

As shown in FIG. 24, in certain embodiments at least a portion of the second shape memory band 2412 is disposed between overlapping portions of the first shape memory band 2410. An artisan will recognize from the disclosure herein, however, that the second shape memory band 2412 may be disposed adjacent to an outer surface or an inner surface of the first shape memory band 2410 rather than between overlapping portions of the first shape memory band 2410. When the second shape memory band 2412 is activated, it expands or contracts so as to slide with respect to the first shape memory band 2410. In certain embodiments, the first shape memory band 2410 and the second shape memory band 2412 are held in relative position to one another by the flexible material and/or suturable material discussed above.

While the first shape memory band 2410 and the second shape memory band 2412 shown in FIG. 24 are substantially flat, an artisan will recognize from the disclosure herein that other shapes are possible including, for example, rod-shaped wire. However, in certain embodiments the first shape memory band 2410 and the second shape memory band 2412 advantageously comprise substantially flat surfaces configured to guide one another during expansion and/or contraction. Thus, the surface area of overlapping portions of the first shape memory band 2410 and/or the second shape memory band 2412 guide the movement of the body member 2400 in a single plane and reduce misalignment (e.g., twisting or moving in a vertical plane) during shape changes. The surface area of overlapping portions also advantageously increases support to a heart valve by reducing misalignment during beating of the heart.

An artisan will recognize from the disclosure herein that certain embodiments of the body member 2400 may not comprise either the first shape memory band 2410 or the second shape memory band 2412. For example, in certain embodiments the body member 2400 does not include the second shape memory band 2412 and is configured to expand and/or contract by only activating the first shape memory band 2410. Further, an artisan will recognize from the disclosure herein that either the first band 2410 or the second band 2412 may not comprise a shape memory material. For example, the first band 2410 may titanium, platinum, stainless steel, combinations of the foregoing, or the like and may be used with or without the second band 2412 to support a coronary valve annulus.

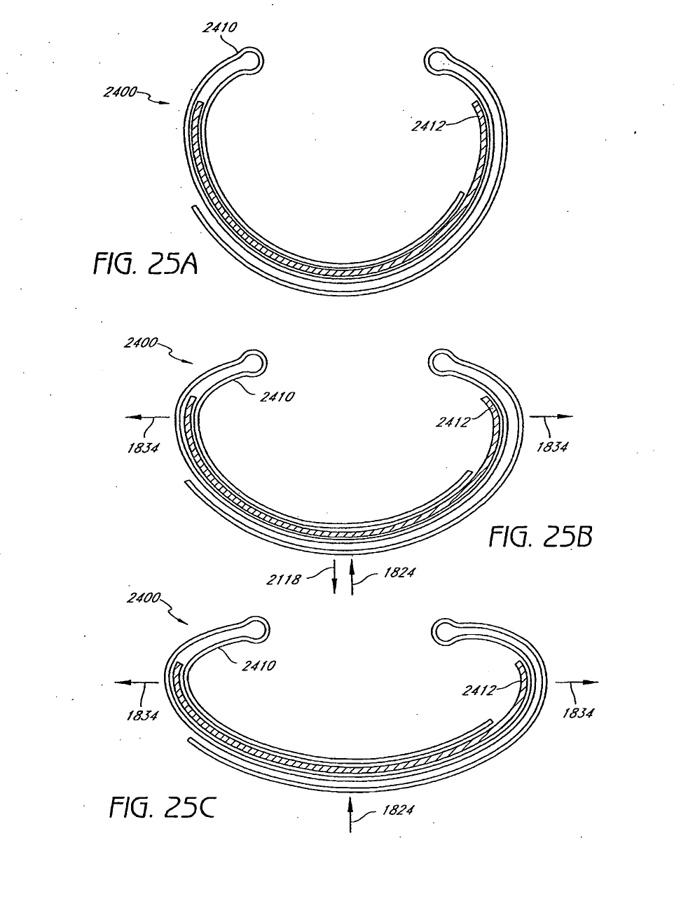

As schematically illustrated in FIGS. 25A-25C, in certain embodiments the body member 2400 is configured to change shape at least twice by activating both the first shape memory band 2410 and the second shape memory band 2412. FIG. 25A schematically illustrates the body member 2400 in a first configuration or shape before the first shape memory band 2410 or the second shape memory band 2412 are activated. In certain embodiments, the first shape memory band 2410 and the second shape memory band 2412 are configured to be activated or return to their respective memorized shapes at different temperatures. Thus, the first shape memory band 2410 and the second shape memory band 2412 can be activated at different times to selectively expand and/or contract the body member 2400. For example (and for purposes of discussing FIGS. 25A-25C), in certain embodiments, the first shape memory band 2410 is configured to be activated at a lower temperature than the second shape memory band 2412. However, an artisan will recognize from the disclosure herein that in other embodiments the second shape memory band 2412 may be configured to be activated at a lower temperature than the first shape memory band 2410.

FIG. 25B schematically illustrates the body member 2400 in a second configuration or shape after the first shape memory band 2410 has been activated. When the first shape memory band 2410 is activated, it responds by bending the body member 2400 in a first direction as indicated by arrow 1824. In certain embodiments, the activation also expands the body member 2400 in a second direction as indicated by arrows 1834. Thus, the body member 2400 is usable by the annuloplasty ring 1826 shown in FIG. 18 to pull the commissures 1818, 1820 further apart and push the leaflets 1812, 1814 closer together to improve coaptation.

In certain embodiments, the second shape memory band 2412 can then be activated to further contract the body member 2400 in the direction of the arrow 1824 and, in certain embodiments, further expand the body member 2400 in the direction of arrows 1834. In certain such embodiments, activating the second shape memory band 2412 reshapes the body member 2400 to a third configuration as shown in FIG. 25C. Thus, for example, as the patient's heart progressively heals and reduces in size, the body member 2400 can be re-sized to provide continued support and improved leaflet coaptation. In certain other embodiments, activation of the second shape memory band 2412 bends the body member 2400 opposite to the first direction as indicated by arrow 2118. In certain such embodiments, activating the second shape memory band 2412 reshapes the body member 2400 to the first configuration as shown in FIG. 25A. Thus, for example, if the size of the patient's heart begins to grow again (e.g., due to age or illness), the body member 2400 can be re-sized to accommodate the growth.

In certain annuloplasty ring embodiments, flexible materials and/or suturable materials used to cover shape memory materials also thermally insulate the shape memory materials so as to increase the time required to activate the shape memory materials through application of thermal energy. Thus, surrounding tissue is exposed to the thermal energy for longer periods of time, which may result in damage to the surrounding tissue. Therefore, in certain embodiments of the invention, thermally conductive materials are configured to penetrate the flexible materials and/or suturable materials so as to deliver thermal energy to the shape memory materials such that the time required to activate the shape memory materials is decreased.

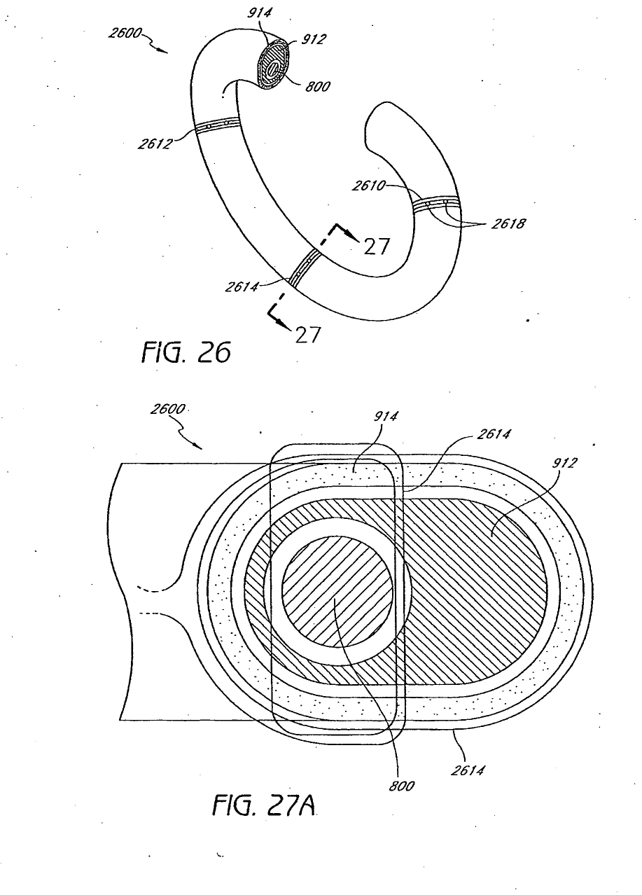

For example, FIG. 26 is a perspective view illustrating an annuloplasty ring 2600 comprising one or more thermal conductors 2610, 2612, 2614 according to certain embodiments of the invention. The annuloplasty ring 2600 further comprises a shape memory wire 800 covered by a flexible material 912 and a suturable material 914, such as the wire 800, the flexible material 912 and the suturable material 914 shown in FIG. 9A. As shown in FIG. 26, in certain embodiments, the shape memory wire 800 is offset from the center of the flexible material 912 to allow more room for sutures to pass through the flexible material 912 and suturable material 914 to attach the annuloplasty ring 2600 to a cardiac valve. In certain embodiments, the flexible material 912 and/or the suturable material 914 are thermally insulative. In certain such embodiments, the flexible material 912 comprises a thermally insulative material. Although the annuloplasty ring 2600 is shown in FIG. 26 as substantially C-shaped, an artisan will recognize from the disclosure herein that the one or more thermal conductors 2610, 2612, 2614 can also be used with other configurations including, for example, circular, D-shaped, or other curvilinear configurations.

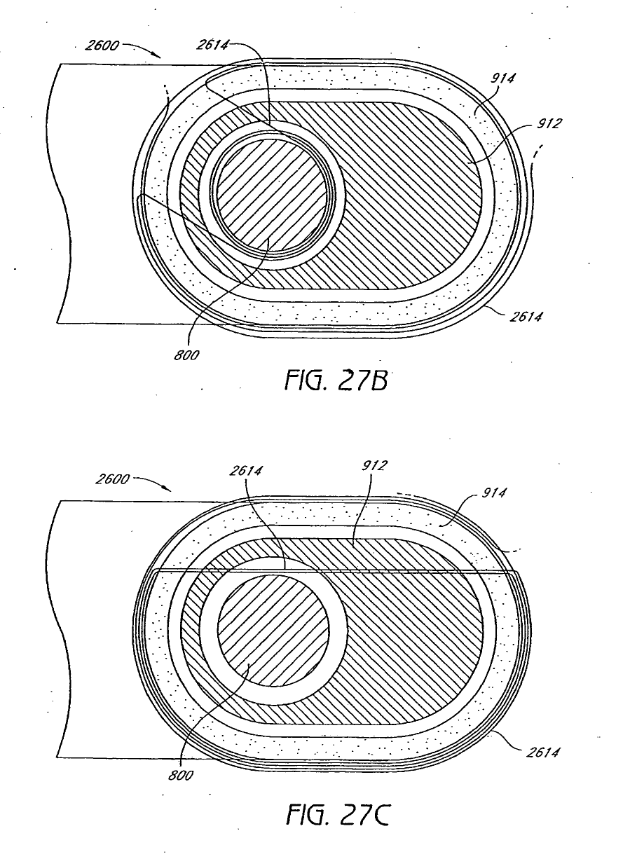

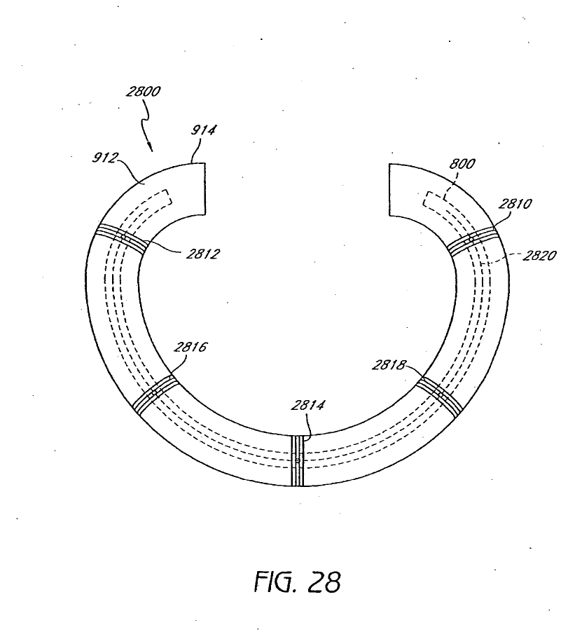

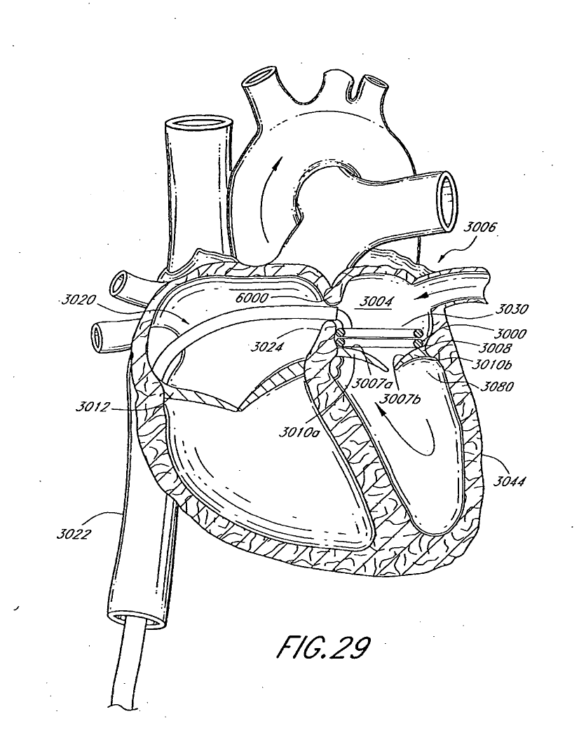

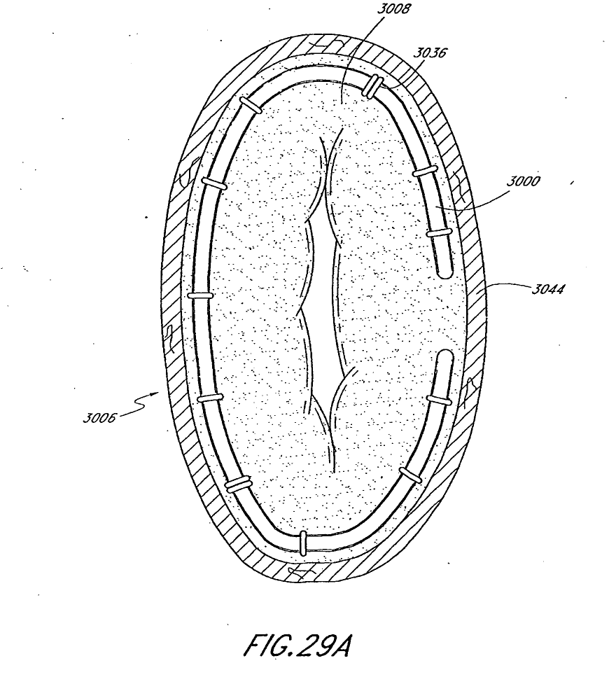

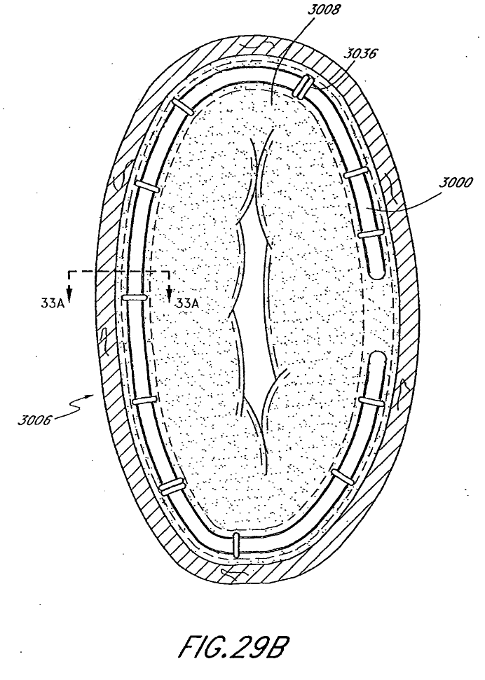

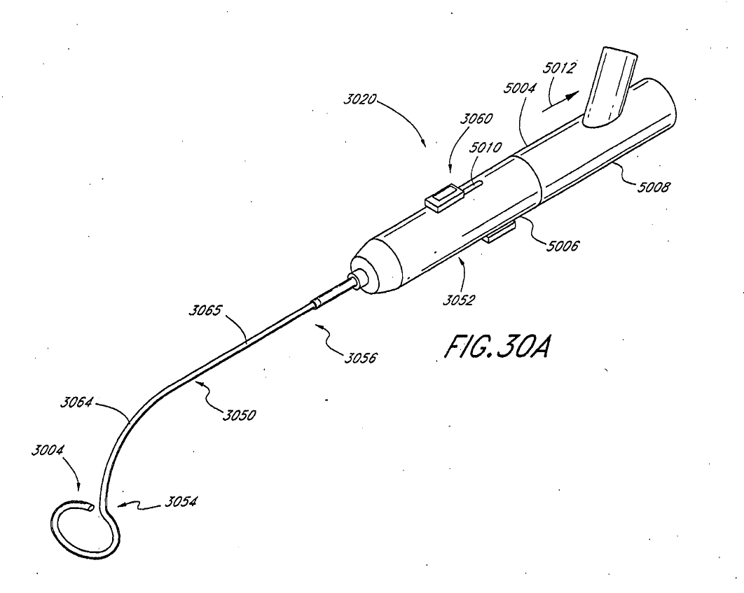

In certain embodiments, the thermal conductors 2610, 2612, 2614 comprise a thin (e.g., having a thickness in a range between approximately 0.002 inches and approximately 0.015 inches) wire wrapped around the outside of the suturable material 914 and penetrating the suturable material 914 and the flexible material 912 at one or more locations 2618 so as to transfer externally applied heat energy to the shape memory wire 800. For example, FIGS. 27A-27C are transverse cross-sectional views of the annuloplasty ring 2600 schematically illustrating exemplary embodiments for conducting thermal energy to the shape memory wire 800. In the exemplary embodiment shown in FIG. 27A, the thermal conductor 2614 wraps around the suturable material 914 one or more times, penetrates the suturable material 914 and the flexible material 912, passes around the shape memory wire 800, and exits the flexible material 912 and the suturable material 914. In certain embodiments, the thermal conductor 2614 physically contacts the shape memory wire 800. However, in other embodiments, the thermal conductor 2614 does not physically contact the shape memory wire 800 but passes sufficiently close to the shape memory wire 800 so as to decrease the time required to activate the shape memory wire 800. Thus, the potential for thermal damage to surrounding tissue is reduced.

In the exemplary embodiment shown in FIG. 27B, the thermal conductor 2614 wraps around the suturable material 914 one or more times, penetrates the suturable material 914 and the flexible material 912, passes around the shape memory wire 800 two or more times, and exits the flexible material 912 and the suturable material 914. By passing around the shape memory wire 800 two or more times, the thermal conductor 2614 concentrates more energy in the area of the shape memory wire 800 as compared to the exemplary embodiment shown in FIG. 27A. Again, the thermal conductor 2614 may or may not physically contact the shape memory wire 800.

In the exemplary embodiment shown in FIG. 27C, the thermal conductor 2614 wraps around the suturable material 914 one or more times and passes through the suturable material 914 and the flexible material 912 two or more times. Thus, portions of the thermal conductor 2614 are disposed proximate the shape memory wire 800 so as to transfer heat energy thereto. Again, the thermal conductor 2614 may or may not physically contact the shape memory wire 800. An artisan will recognize from the disclosure herein that one or more of the exemplary embodiments shown in FIGS. 27A-27C can be combined and that the thermal conductor 2614 can be configured to penetrate the suturable material 914 and the flexible material 912 in other ways in accordance with the invention so as to transfer heat to the shape memory wire 800.

Referring again to FIG. 26, in certain embodiments the locations of the thermal conductors 2610, 2612, 2614 are selected based at least in part on areas where energy will be applied to activate the shape memory wire 800. For example, in certain embodiments heat energy is applied percutaneously through a balloon catheter and the thermal conductors 2610, 2612, 2614 are disposed on the surface of the suturable material 914 in locations likely to make contact with the inflated balloon.

In addition, or in other embodiments, the thermal conductors 2610, 2612, 2614 are located so as to mark desired positions on the annuloplasty ring 2600. For example, the thermal conductors 2610, 2612, 2614 may be disposed at locations on the annuloplasty ring 2600 corresponding to commissures of heart valve leaflets, as discussed above with respect to FIG. 18. As another example, the thermal conductors 2610, 2612, 2614 can be used to align a percutaneous energy source, such as a heated balloon inserted through a catheter, with the annuloplasty ring 2600. In certain such embodiments the thermal conductors 2610, 2612, 2614 comprise radiopaque materials such as gold, copper or other imaging materials, as is known in the art.

FIG. 28 is a schematic diagram of an annuloplasty ring 2800 according to certain embodiments of the invention comprising one or more thermal conductors 2810, 2812, 2814, 2816, 2818, such as the thermal conductors 2610, 2612, 2614 shown in FIG. 26. As schematically illustrated in FIG. 28, the annuloplasty ring 2800 further comprises a shape memory wire 800 covered by a flexible material 912 and a suturable material 914, such as the wire 800, the flexible material 912 and the suturable material 914 shown in FIG. 9A.

In certain embodiments, the shape memory wire 800 is not sufficiently thermally conductive so as to quickly transfer heat applied in the areas of the thermal conductors 2810, 2812, 2814, 2816, 2818. Thus, in certain such embodiments, the annuloplasty ring 2800 comprises a thermal conductor 2820 that runs along the length of the shape memory wire 800 so as to transfer heat to points of the shape memory wire 800 extending beyond or between the thermal conductors 2810, 2812, 2814, 2816, 2818. In certain embodiments, each of the thermal conductors 2810, 2812, 2814, 2816, 2818, comprise a separate thermally conductive wire configured to transfer heat to the thermal conductive wire 2820. However, in certain other embodiments, at least two of the thermal conductors 2810, 2812, 2814, 2816, 2818 and the thermal conductor 2820 comprise one continuous thermally conductive wire.

Thus, thermal energy can be quickly transferred to the annuloplasty ring 2600 or the annuloplasty ring 2800 to reduce the amount of energy required to activate the shape memory wire 800 and to reduce thermal damage to the patient's surrounding tissue.

The adjustable rings described above can be implanted in the heart to improve the efficacy of the heart. For example, one or more adjustable rings can be implanted in the heart to improve the function (e.g., leaflet operation) of a heart valve. Adjustable rings can help reduce or prevent reverse flow or regurgitation while preferably permitting good hemodynamics during forward flow. Of course, the adjustable rings can be employed for other treatments.

After a treatment period, the efficacy of the heart may degrade, or the heart may be ready to undergo further treatment. At some point after implantation of the adjustable ring, the adjustable ring can be activated to change its configuration (e.g., its shape). The adjustable ring can be activated minutes, hours, days, months, and/or years after implantation. In some embodiments, the adjustable ring can be activated immediately after the adjustable ring is implanted into the patient. The adjustable ring may be activated one or more times depending on the particular treatment. A physician can perform tests, as are known in the art, to determine if the patient should undergo further treatment after implantation of the ring.

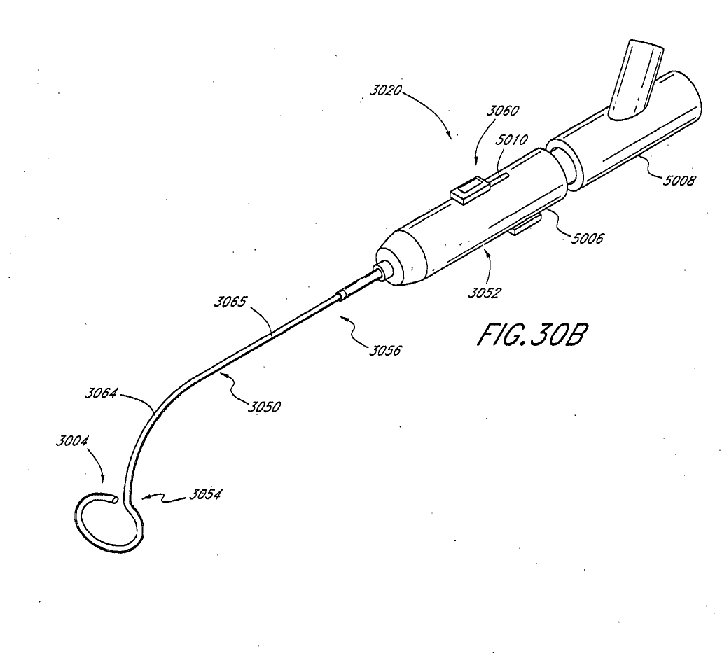

FIG. 29 illustrates a device implanted in a heart to improve functioning of the heart. A catheter system 3020 is positioned within a heart 3006 and can be used to adjust the shape of the implantable device 3000, and thus the shape of the heart. The illustrated implantable device 3000 is an adjustable annuloplasty device implanted in the left atrium 3004 of the heart 3006. When the implantable device 3000 is positioned in the patient's body, the catheter system 3020 can be used to activate the implantable device 3000 in situ. When the catheter system 3020 is delivered to the heart, the catheter system 3020 is configured to activate the implantable device 3008. In some embodiments, energy emitted from a distal element 3030 of the catheter system 3020 can activate the implantable device 3000.

A mitral valve 3008 can be treated by the implantable device 3000. The illustrated implantable device 3000 is disposed on an upper side 3007a of the anterior leaflet 3010a and an upper side 3007b of the posterior leaflet 3010b of the mitral valve 3008. However, it is contemplated that the implantable device 3000 can be positioned on the lower side of the leaflets 3010a, 3010b. For example, the implantable device 3000 can be positioned in the left ventricle. In some non-limiting embodiments, the device 3000 is snaked through the chordae tendineae and then is placed against the lower surfaces of the leaflets 3010a, 3010b. Alternatively, the chordae tendineae can be cut to provide a delivery path for implantation of the implantable device 3000. In certain embodiments, the implantable device 3000 can also be implanted at other locations in the vasculature system, or at any other position within a patient's body. For example, the implantable device 3000 can be implanted at a location proximate to the tricuspid valve 3012. The implantable device 3000 can be positioned on the upper side or lower side of the tricuspid valve 3012 to improve the efficacy of the tricuspid valve 3012.

The catheter system 3020 can be used to affect a single implantable device or a plurality of implantable devices implanted in the vasculature system. During a single surgical procedure, the catheter system 3020 can thus be operated to adjust the shape of any number of implantable devices 3000 positioned any position the patient's body. For example, a plurality of implantable devices can be implanted in a patient's heart to enhance the function of the heart's valves. Each of the implantable devices can be activated by the catheter system 3020. For the sake of convenience, only some exemplary catheter systems and methods of activating the implantable devices at certain locations are described in detail. However, an artisan will recognize that the implantable devices can be implanted at other locations and can be activated by using the catheter systems and methods described herein.

The catheter system 3020 can be delivered through the vascular system to engage the implantable device 3000. As such, the length and diameter of the system 3020 can be selected to permit percutaneous entry into the vascular system and, preferably, transluminal advancement through the vascular system to an implantation site.

To activate the implantable device 3000 which is proximate to the mitral valve 3008, the catheter system 3020 can be positioned through the heart 3006, as illustrated. The illustrated catheter system 3020 extends upwardly through the inferior vena cava 3022 to the heart 3006 via the right atrium, a hole in an interatrial septum 3024 and into the left atrium 3004. The catheter system 3020 comprises a distal element 3030 that is in operative engagement with the implantable device 3000. The distal element 3030 is used to activate and adjust the preferential shape of the implantable device 3000, preferably to improve the efficacy of the mitral valve 3008. For example, if the leaflets 3010a, 3010b of the mitral valve 3008 are somewhat misaligned, even though the implantable device 3000 is implanted, the implantable device 3000 can be activate to bring the leaflets 3010a, 3010b into proper alignment. Non-limiting exemplary implantable devices can be used to change (e.g., to increase or decrease) the circumference of the mitral valve 3008, thus causing the valve leaflets 3010a, 3010b attached to the annulus to close more completely, thereby restoring normal and effective valve operation. As such, the mitral valve 3008 can properly regulate of flow from the left ventricle to the left atrium.

With continued reference to FIG. 29, the implantable device 3000 is securely attached to the tissue of the heart 3006. One or more coupling structures, such as sutures, staples, adhesives, and the like, can coupled the implantable device 3000 to the heart tissue. In the illustrated embodiment of FIGS. 28 and 29, the implantable device 3000 is attached to the heart tissue by a plurality of coupling structures 3036 in the form of sutures spaced, evenly or unevenly, along the length of the implantable device 3000. The number and positions of the sutures 3036, configuration of the device 3000, and implantation site can be selected to enhance the opening and closing of the mitral valve 3008. As the mitral valve 3008 regulates blood flow, the implantable device 3000 can remain securely fixed in place.

The coupling structures can be positioned at any suitable location in the heart. The coupling structures 3036 can be attached to the leaflets 3010a, 3010b, the sidewall of the left atrium 3004, the septal wall, and/or any other tissue of the heart. The illustrated implantable device 3000 is positioned near or at the junction of the leaflets 3010a, 3010b and the sidewall 3044 of the heart 3006 (FIG. 28). In some embodiments, the implantable device 3000 is attached to the annulus (i.e., the fibrous ring comprising tough fibrous tissue) of the mitral valve. Sutures can extend through the annulus to securely hold the implantable device 3000 in place. As such, the leaflets 3010a, 3010b are relatively unobstructed and can freely flap downwardly and upwardly. Of course, the position of the implantable device 3000 and sutures can be selected based on the anatomy of the patient and the particular treatment.

When the implantable device 3000 is implanted, the implantable device 3000 preferably protrudes from the body tissue to facilitate engagement with the distal element 3004. The distal element 3004 can be laid upon the exposed device 3000. Alternatively, the implantable device 3000 can be partially or completely embedded in the tissue of the patient.

When the distal element 3004 of the catheter system 3020 is positioned near or proximate to the implantable device 3000, the distal element 3004 can be somewhat vertically aligned with the implantable device 3000. As shown in FIG. 29B, the distal elements 3004 (shown in phantom) of the catheter system 3020 can overlay the implantable device 3000.

FIG. 30A is a perspective view of the catheter system 3020. The catheter system 3020 preferably comprises the distal element 3004, a steerable shaft assembly 3050, and a handle assembly 3052. Generally, when the distal element 3004 operatively engages the implantable device 3000, as shown in FIG. 29, the catheter system 3020 can be used to deliver energy (e.g., thermal energy) suitable to activate the implantable device 3000. In particular, when the distal element 3004 touches or is in the vicinity of an implantable device, the distal element 3004 can heat and change the shape of the implantable device 3000. Heated media can be circulated through catheter system 3020, including the handle assembly and the steerable shaft assembly 3050, to heat the distal element 3004. Preferably, the heated media circulates through the distal element 3004 so that heat is transferred to the implantable device 3000.

The shape of the distal element 3004 can be controllably selected by operating the handle assembly 3052. The illustrated handle assembly 3052 can be actuated between a first sizing position (FIG. 30A) and a second sizing position (FIG. 30B) for adjusting the shape and configuration of the distal element 3004.

The illustrated distal element 3004 of FIG. 30A is disposed at a distal end 3054 of the shaft assembly 3050. The handle assembly 3052 is connected to a proximal end 3056 of the shaft assembly 3050. A control system 3060 of the handle assembly 3052 can be used to move controllably the distal element 3004 by steering the shaft assembly 3050.

The shaft assembly 3050 can be somewhat flexible and preferably has a first portion 3064 that can be articulated to move the distal element 3004. In FIG. 30A, the first portion 3064 is curved radially outward in the distal direction, although the first portion 3064 can be articulated in other directions as desired. As shown in FIG. 31, the first portion 3064 can be articulated between a first position 4050 and a second position 4052. The first portion 3064 occupying the first position 4050 and the second position 4052 is shown in phantom.

FIG. 32 is a longitudinal cross-sectional view of the catheter system 3020 of FIG. 31. Various components of the shaft assembly 3052 of the catheter system 3020 according to some embodiments of the invention are illustrated and described in connection with FIG. 43 below. 32A-32A of FIG. 32 highlights the distal element 3004 of the catheter system 3020, which is shown enlarged in an end view in FIG. 32A. 32B-32B of FIG. 32A highlights the core portion 3074 of the distal element 3004, which is shown enlarged in FIG. 32B. Various components of the distal element 3004 including the core portion 3074 according to some embodiments of the invention are illustrated and described in connection with FIG. 35 below.

FIG. 33 is an enlarged perspective view of the distal element 3004 that is configured to operatively mate with the implantable device 3000 in situ. The distal element 3004 can have a similar or identical configuration as the implantable device 3000. Thus, the distal element 3004 can be selected from one or more shapes comprising a round or circular shape, an oval shape, a C-shape, a D-shape, a U-shape, an open circle shape, an open oval shape, curvilinear shapes, or other configurations based on the configuration of the implantable device 3000. As such, the distal element 3004 can be matched with and laid upon the implantable device 3000. For example, as shown in FIGS. 29 and 29B, the distal element 3004 (shown in phantom in FIG. 29B) can be placed upon the implantable device 3000 has a generally open oval shape. The shape of the distal element 3004 can be selected for a desired amount of energy to be delivered to the implantable device 3000 during the activation process. The illustrated distal element 3004 forms a generally annular body that terminates at a tip 3069. When referred to herein, a substantially annular shape may include substantially circular, elliptical, and oval shapes.

With reference again to FIG. 33, the distal element 3004 includes a balloon member 3070 and a core 3074 (shown in phantom) extending through the balloon member 3070. A chamber 3080 (see FIG. 34) can be defined between the balloon member 3070 and the core 3074. The balloon member 3070 can be controllably expanded (e.g., inflated) by filling the chamber 3080 with a working media.

The balloon member 3070 can conform to the shape of the implantable device 3000. The balloon member 3070 can be any suitable expandable member for activating the implantable device. The illustrated balloon member 3070 is in the form of an inflatable balloon member that can be inflated to increase the surface area of the distal element 3004. The increased surface area can improve engagement of the distal element 3004 with the implantable device 3000. The balloon member 3070 preferably extends along a substantial portion of the distal element 3004.

The illustrated balloon member 3070 includes an outer membrane inflatable between a collapsed position and an inflated position and can be constructed from a variety of materials. In some embodiments, the balloon member 3070 comprises a compliant and/or a non-compliant polymer material. If the balloon member 3070 comprises compliant material, the compliant material can deform (e.g., stretch) upon the application of pressure. The balloon member 3070 can therefore conform to the shape of the implantable device 3000 when the balloon member 3070 is at least partially inflated and pressed against the implantable device 3000. Advantageously, the compliant balloon member 3070 can form an atraumatic surface to reduce injuries to the patient.

The balloon member 3070 can comprise one or more polymers, such as polyester, silicone, polyurethane, latex, combinations thereof, and other suitable materials for forming a balloon. The balloon member 3070 may comprise any combination of materials with any thicknesses, depending upon the desired functional result. In some embodiments, the balloon member 3070 comprises a monolayer or multilayer membrane. At least one layer of the membrane can be a barrier layer that inhibits, preferably substantially prevents, the egress and/or ingress of media.

In some embodiments, the balloon member 3070 comprises a woven or braided polymer incorporating filaments or wires. For example, the balloon member 3070 can comprise a polymer surrounding braided metallic wires made of stainless steel, platinum, gold, or other suitable materials forming at least part of the balloon member 3070. As such, the balloon member 3070 can withstand high pressures and can have a preset maximum inflated position. The filament or wires can also increase thermal and/or mechanical properties of the distal element 3004.

If the balloon member 3070 has wires (or filaments), the wires can be attached to its interior surface, woven through the balloon member 3070, attached to the outer surface of the expandable member, and/or at any other suitable location. For example, the wires can be affixed to the interior surface of the balloon member 3070 and can form at least a portion of the chamber 3080. In other embodiments, the wires can be woven through the balloon member 3070 so that the wires form part of the outer surface and the interior surface of the expandable member. Exemplary balloon members can have wires can have a high thermal conductivity to rapidly transfer heat through the balloon member 3070. In other embodiments, the metal wires are wrapped around the outer surface of the balloon member 3070.

The balloon member 3070 can have other structures to enhance heat transfer from the media in the chamber 3080 to the implantable device 3000. Particles, strands, filaments, wires, additives, and/or other means for promoting heat transfer to the implantable device 3000.

In some embodiments, one or more materials can be incorporated into the material forming the balloon member 3070 to enhance heat transfer. The balloon member 3070 can be doped or treated with an additive material that enhances thermal performance of the catheter system 3020, for example. A material having a high thermal conductivity can be added to the balloon member 3070 to enhance heat transfer between a heated media in the chamber 3080 and the implantable device 3000. In some embodiments, the balloon member 3070 comprises a polymer that includes one or more additives (e.g., dopants such as thermally conductive additives like powdered metals). The dopant can be dispersed evenly or unevenly throughout a membrane forming the balloon member 3070.

Additives known by those of ordinary skill in the art for their ability to provide enhanced fluid barrier, thermal conductivity, chemical resistance thermal properties, and/or structural properties may be used. Preferred additives may be prepared by methods known to those of skill in the art. For example, the additives may be mixed directly with a particular polymer during the membrane manufacturing process. In addition, in some embodiments, preferred additives may be used alone as a single coating layer. The coating layer can be formed on the interior surface, exterior surface, or any other desirable location of the balloon member 3070.

FIG. 35 is a cross-sectional view of the distal element 3004 in operative engagement with the implantable device 3000 in situ. The balloon member 3070 is movable radially from a first position (e.g., a collapsed position) to a second position (e.g., a partially or fully inflated position). When the balloon member 3070 occupies the second position, the chamber 3080 is defined between an interior surface 3092 of the balloon member 3070 and the exterior surface 3094 of the core 3074.

The width W of the chamber 3080 defined between the core 3074 and the balloon member 3070 can be increased or decreased by increasing or decreasing, respectively, the pressure within the chamber 3080. Preferably, the balloon member 3070 is at least partially inflated when the exterior surface 3098 of the balloon member 3070 engages the implantable device 3000. The distal element 3004 can be pressed against the implantable device 3000 and can conform to the shape of the implantable device 3000 so as to provide an increased contact area between the balloon member 3070 and the implantable device 3000. Alternatively, the balloon member 3070 can be spaced from the implantable device 3000, when the balloon member 3070 activates the implantable device 3000. As such, the balloon member 3070 can be made of a substantially non-compliant material.

With reference again to FIG. 33, the core 3074 can extend through a passageway defined by the balloon member 3070. The core 3074 can provide structural support to the balloon member 3070 to help maintain the shape of the distal element 3004 and can be configured to inflate and deflate selectively the balloon member 3070. To expand the balloon member 3070, media can be delivered along the shaft assembly 3050 to the distal element 3004 to fill and inflate the balloon member 3070. To deflate the balloon member 3070, media can flow in the reverse direction through the catheter system 3020.

With continued reference to FIG. 33, one or more ports can be positioned along the core 3074 for selectively inflating and/or deflating the balloon member 3070. In some embodiments, including the illustrated embodiment, the core 3074 can comprise at least one inlet port 3381 and at least one outlet port 3076 that are in communication with the chamber 3080. The inlet port 3381 and the outlet port 3076 can cooperate to define a fluid path through the chamber 3080.

As shown in FIG. 34, media can flow through a core delivery lumen 3084 towards the distal element 3004. Media delivered through the shaft assembly 3050 can flow out of the inlet port 3381 and into the chamber 3080. Additionally, the media can then flow through the chamber 3080 and back into the core 3074 via the outlet port 3076. The amount of media circulating through the chamber 3080 can be selected based on the desired level of inflation. The media can flow continuously or intermittently through to the chamber 3080 to achieve the desired level of inflation, temperature of the distal element 3004, and the like.

The core 3074 comprises a plurality of lumens. The illustrated core 3074 of FIG. 35 comprises a core lumen 3084 configured to provide fluid communication between the inlet port 3381 and the outlet port 3076. As shown in FIGS. 34 and 35, the core lumen 3084 is a fluid passageway that extends from the inlet port 3381 to the outlet port 3076.

A control lumen 4002 of the core 3074 houses and surrounds a control wire 4006. The control wire 4006 can extend distally from the tip 3069 through the control lumen 4002 and the shaft assembly 3000. The control wire 4006 can be tensioned to adjust a size and configuration of the distal element 3004. For example, when the distal element 3004 is at rest, the control wire 4006 can be pulled proximally to reduce the diameter of the distal element 3004. When the control wire 4006 is not tensioned, the core section 3074 can be biased to a preset at rest configuration. Of course, the proximal force applied to the wire can change the curvature of the distal element 3004. Thus, the control wire 4006 can be actuated to move the distal element between one or more configurations.

The distal element 3004 can include one or more sensors configured to detect one or more of the following: pressure, temperature, media flow rate, and combinations thereof. The sensors can send signals indicative of a detected value. The illustrated core 3074 comprises a sensor 4010 configured to measure indirectly the temperature of the working media within the chamber 3080, although the sensor 4010 can be positioned to measure the temperature of the working media directly. The sensor 4010 can be positioned at any suitable point along the core 3074. In the illustrated embodiment, the sensor 4010 is positioned generally near the exterior surface 3094 and can measure rapid temperature changes of the working media. In certain embodiments, the temperature of the working media in the chamber 3080 can be calculated based on the measurements taken by several sensors 4010, each disposed within the core 3074. The temperature sensors 4010 can be embedded within the core 3074, attached to the exterior surface 3094 of the core 3070, attached to the inner surface 3092 of the balloon member 3070, or at any other suitable position for measuring the temperature of the distal element 3004.

A plurality of sensors 4010 can be positioned at various points along the long axis of the core 3074. The sensors 4010 can be used to monitor the temperature at one or more locations to predict the amount of energy emitted from the distal element 3004. In some embodiments, the sensors 4010 are spaced along the core 3074 and measure the temperature of the working media throughout the entire length of the distal member 3004.

The sensors 4010 can also measure the temperature of the balloon member 3070, blood surrounding the distal element 3004, the implantable device 3000, or any other temperature of interest. In some embodiments, for example, one or more sensors 4010 can be positioned on the exterior surface of the balloon member 3070.

The core 3074 can have one or more lumens that carry lead wires, such as thermocouple lead wires, that are in communication with a controller. Any suitable communication means can be employed to provide communication between the sensors 4010 and another device, such as a controller.

In operation, to heat the distal element 3004, a preheated media can be passed through the shaft assembly 3050 (FIG. 30A) and to the distal element 3004. The media can be preheated by a heating system that can selectively control the temperature of the media. The temperature of the implantable device 3000 is typically at or near the temperature of the surrounding tissue. The temperature of the media can be sufficiently high such that an effective amount of heat is transferred from the media through the balloon membrane 3070 and to the implantable device 3000. The implantable device 3000 can absorb heat until it is activated.

The heated media can flow to the outlet port 3076 and into the core 3074. The heated media can then flow out of the distal element 3004 and proximally through the shaft assembly 3050. In this manner, heated media can circulate through the distal element 3004 to continuously transfer heat to the implantable device 3000. In alternative embodiments, the heated media can flow into and fill the balloon member 3070. When the balloon member 3070 is filled, the flow is stopped. The heating media, in a static condition, can transfer heat through the balloon member 3070 to the implantable device 3000 in order to elevate the temperature of the implant device 3000. In other embodiments, the heated media can be pulsed through the distal element 3004 to provide periodic or cyclic heating of the distal element 3004 and associated implantable device 3000.

As used herein, the term “media” is a broad term and is used in its ordinary meaning and may include, without limitation, a flowable substance that can be heated or cooled as desired. In some embodiments, the media can comprise water, saline, flowable gel, and other suitable materials that can be delivered to the distal element 3004. The media can comprise a sterilized material so that if the balloon member 3070 ruptures, a sterilized material will be delivered into the bloodstream. The temperature of the media delivered to the distal element 3004 can be selected based upon the desired surface temperature of the balloon element 3070. In some embodiments, the temperature of the media is about 37° C., 40° C., 42° C., 44° C., 46° C., 48° C., 50° C., 52° C. and ranges encompassing such temperatures.

Advantageously, the temperature of the media can be reduced if the media is dynamically flowing through the distal element 3004, as compared to media in a static condition. Thermal energy is transferred from the heated media through the balloon member 3070 and is transferred via conduction and/or convection to the implantable device 3000. Of course, the temperature of the media can be higher than the activation temperature of the implantable device 3000 so that the temperature of the implantable device 3000 will be raised to its preset activation temperature.

In some non-limiting exemplary embodiments, the preset activation temperature of the implantable device 3000 is equal to or greater than about 38° C., 40° C., 42° C., 44° C., 46° C., 48° C., 50° C., 52° C., 54° C., 56° C., 58° C., 60° C., and ranges encompassing such temperatures. Preferably the temperature of the media is greater than the activation temperature of the implantable device by more than about 1° C., 2° C., 3° C., 4° C., 5° C., 6° C., 7° C., 8° C., 9° C., 10° C., 11° C., and ranges encompassing such temperature differences. In one embodiment, the activation temperature of the implantable device 3000 is about 40° C. The implanted implantable device 3000 can be at a temperature of about 38° C. The distal element 3004 can be heated to a temperature greater than 43° C. and used to heat the implantable device 3000 to a temperature greater than 40° C. to change the configuration of the implantable device 3000. The temperature of the distal element 3004 can be selected based on the activation temperature of the implantable device 3000, the desired activation period, and other treatment parameters.

In addition to the passive process of heating the distal element 3004 by a heated media, the distal element can be actively heated. For example, the distal element of the catheter system can have one or more active thermal energy sources that can be employed with or without utilizing passive heating. The thermal energy source(s) used to heat the distal element 3004 can comprise one or more of the following: resistors, transducers, lasers, media, and combinations thereof.

FIG. 36 illustrates a distal element 4020 that comprises one or more active thermal energy sources (e.g., electrical resistors, heaters) that can be actively heated. In some embodiments, the distal element 4020 has an electrical resistor in the form of coils 4022 that surround an inner core 4024. The coils 4022 can comprise one or more electrical conduits (e.g., wires) that are heated when an electrical current is applied thereto. The electrical conduits can be wrapped around the core 4024 in a spiral manner, or other configuration, and can extend the length of the core 4024. However, the coils 4022 can also be embedded within the core 4024, although the coils can be at other locations suitable for transferring heat to the implantable device 3000. The coils 4022 can be used before, during, and/or after the balloon element 3070 is inflated. In some embodiments, including the illustrated embodiment of FIG. 37, the coils 4022 form at least a portion of the chamber 3080. When a media (preferably a thermally conductive media) fills the chamber 3080, the media can transfer heat from the coils 4022 to the exterior surface of the distal element 4020. The media is then heated as the media flows across the coils 4022 for rapid heat transfer. Thus, the media can be heated in the distal element 3004. Of course, preheated media can be delivered to the distal element 3004 even if the coils 4022 are employed. The coils 4022 can comprise tungsten, gold, copper, silver, aluminum, combinations thereof, or other materials suitable for forming a heating element.

With respect to FIG. 38, the distal element 4036 comprises one or more bands positioned along its length to provide heating capabilities. The illustrated distal element 4036 comprises a plurality of heating elements 4037 spaced from each other along the length of the distal element 4037. One or more of coils, bands, or partial bands (e.g., quarter bands, half bands, etc.), can form the heating elements 4037. The illustrated heating elements 4037 are in the form of a pair of annular bands that surround the balloon member 3070.

As shown in FIG. 39, the bands 4037 can extend about the periphery of the balloon member 3070. Alternatively, the bands 4037 can be woven in and out of the balloon member 3070 to enhance heat transfer from the media within the balloon 3070 to the implantable device 3000. However, the bands can be at other locations for any desired heating functionality. The bands 4037 can comprise copper, gold, metal alloys, polymers, and other suitable materials for transferring heat, preferably transferring heat rapidly.

With respect to FIG. 40A, a distal element 4034 can have one or more positioning structures 4012 for facilitating seating to the implantable device 3000. The illustrated positioning structure 4012 is a deployable elongated projection that extends outwardly from the balloon member 4013. The structure 4012 can engage the wall of the heart, the implantable device 3000, or other structure to facilitate positioning of the element 4034. The balloon member 4013 can have sufficient mechanical properties so that the positioning member 4012 will extend outwardly during use. As the distal element 4034 passes through a deliver device (e.g., a delivery sheath), the positioning structures 4012 can be collapsed for a low profile delivery configuration. The positioning structure 4012 can be deployed outwardly as the distal element 4034 is delivered out of the delivery sheath. The positioning structures 4012 can help capture the implantable member 3000 and locate the distal element 4034.

The distal elements can have other position structure(s) to aid in mating of the distal element to the implantable device. FIG. 40B illustrates a distal element 4038 that has a preferential preset configuration. The illustrated balloon member 4039, when inflated, has a positioning structure in the form of recessed region 4041. The recessed region 4041 can define a channel that is sized and configured to receive a least a portion of the implantable device 3000. The implantable member 3000 can be received and contained in the recessed region 4041. Other types of positioning structure(s) (e.g., protrusions, locators, etc.) can be employed to facilitate engagement of the distal element 4038 with the implantable member 3000. When the distal element outputs energy, the positioning structures can help maintain operative engagement of the distal element and the implantable member 3000.

With reference again to FIG. 30A, the steerable shaft assembly 3050 cooperates with the handle system 3052 for controllable deflection and steering of the distal element 3004. The first portion 3064 extends distally from the second portion 3065 of the shaft assembly 3050. The second portion 3065 is preferably somewhat stiffer than the first portion 3064. In some embodiments, the second portion 3056 surrounds the first portion 3064. As such, when the distal element 3004 is maneuvered and delivered to a desired site, the first portion 3064 can bend and deflect easier than the proximal portion 3065. The second portion 3056 and the first portion 3064 can be relatively flexible to facilitate delivery through a delivery sheath that is positioned along a somewhat tortuous path.

The second portion 3065 can be braided or non-braided tubing that can have one or more central lumens depending on the function of the catheter system 3020. The illustrated second portion 3065 comprises a single lumen 4030 that is sized to house the first portion 3064 extending therethrough.

With reference to FIGS. 41 and 42, the shaft assembly 3050 can comprise one or more pull wires that are used for moving the distal element 3004. The illustrated shaft assembly 3050 can comprise a first pull wire 4032 and a second pull wire 4034. The pull wires 4032, 4034 extend proximally from pins 4042, 4044 (FIG. 41), respectively, to the control system 3060 (FIG. 30A). The pins 4042, 4044 are preferably at the distal end 4045 of the first portion 3064.

As used herein, the term “pull wire” is intended to include any of a wide variety of structures which are capable of transmitting axial tension and/or compression, such as a pushing or pulling force sufficient move the distal element 3004. In some embodiments, the pull wires 4032, 4034 can be a polymer wire, woven or braided structure, metal wire, mono-filament or multi-filament wire, and the like. The pull wires can have a solid or hollow cross-section. For example, in some embodiments the pull wires can be tubes that are axially, movably disposed in the shaft assembly 3050. In the illustrated embodiment of FIG. 42, the shaft assembly 3050 has a first pull wire lumen 4046 and a second pull wire lumen 4048. The first pull wire 4032 and the second pull wire 4034 are positioned within the first pull wire lumen 4046 and the second pull wire lumen 4048, respectively. Each of the pull wires 4032, 4034 extends proximally throughout the length of the shaft assembly 3050 to the handle assembly 3052.

In the illustrated embodiments of FIGS. 41 and 42, the first pull wire 4032 can be pulled proximally without pulling the second pull wire 4034, such that the shaft assembly 3050 is moved to the first position 4050, as shown in FIG. 31. Only the second pull wire 4034 is tensioned such that the shaft assembly 3050 is moved to the second position 4052, as shown in FIG. 31. Thus, a proximally directed force can be applied to at least one of the pins 4042, 4044 to cause bidirectional movement of the shaft assembly 3050. In some embodiments, the first pull wire 4032 and the second pull wire 4034 can apply a distal and proximal force to the pins 4042, 4044 to actuate the distal element 3004. For example, the first pull wire 4032 can be a generally stiff member that is used to apply a distally directed force to the pin 4042. The first pull wire lumen 4046 can surround and prevent buckling of the pull wire 4032 when the pull wire 4032 applies a distally directed force. The second pull wire 4034 can apply a proximally directed force to the pin 4044 as described above. Thus, the pull wires 4032, 4034 can be used to push and pull on the distal end 3054 of the shaft assembly 3050 to achieve the desired movement. Any number of pull wires and associated lumens can be employed for the desired steerability.

With continued reference to FIG. 42, the shaft assembly 3050 can comprise one or more fluid lumens configured to provide fluid communication with the distal element 3004. The shaft assembly 3050 comprises a delivery lumen 4060 and a return lumen 4062. The delivery lumen 4060 and the return lumen 4062 extend along the length of the shaft assembly 3050 and are in communication with the inlet port 3381 and the outlet port 3076, respectively. Media can be passed distally through the delivery lumen 4060 and eventually through the inlet port 3381. After the media has flown through the balloon element 3070, the media can flow out of the outlet port 3076 and into the return lumen 4062. The media can then flow proximally through the return lumen 4062 to the handle assembly 3052 and ultimately through the outlet port 3381.

The shaft assembly 3050 preferably comprises a control lumen 4066 that surrounds a control wire 4068. The control wire 4068 can extend distally from the handle assembly 3052 and through the length of the shaft assembly 3050 to the distal element 3004. In some embodiments, the distal end of the control wire 4068 is connected to the tip 3069. The control wire 4068 can be pulled proximally and/or pushed distally to adjust the size of the distal element 3004. In some embodiments, the control wire 4068 can be used to contract and expand the distal element 3004 between a plurality of configurations. When the control wire 4068 is pulled proximally, the distal element 3004 can be moved inwardly. The distal element 3004 can be biased to a preset configuration and may provide resistance when the wire 4068 is moved, thereby providing tactile feedback to the user. In some embodiments, the control wire 4068 can be actuated distally to move the distal element 3004 outwardly to a first configuration to increase the loop size of the distal element 3004. The control wire can be actuated proximally to move the distal element 3004 to a second configuration to reduce the loop size of the distal element 3004. The control wire 4068 can therefore be used to actuate the distal element 3004 as desired.

With reference again to FIGS. 30A and 43, the handle assembly 3052 has the control system 3060 for moving the distal element 3004. The operator can manually use the control system 3060 for steering the shaft assembly 3050 in one or more directions. The illustrated control system 3060 is designed for bidirectional deflection of the shaft assembly 3050, although the control system 3060 can be modified so that the shaft assembly 3050 is deflected in any number of directions.

The illustrated control system 3060 has a pair of knobs 5000, 5002 that can be actuated to cause bidirectional movement of the distal element 3004 (FIG. 31). The handle assembly 3052 preferably comprises a housing 5004. The control system 3060 and the housing 5004 can thus be used in combination to position and/or adjust the size of the distal element 3004.

The illustrated housing 5004 includes a distal housing portion 5006 and a proximal housing portion 5008 that can be moved relative to each other to selectively adjust the configuration of the distal element 3004. The proximal housing portion 5008 can move from an initial position (FIG. 30A) to a second position (FIG. 30B). As the proximal housing portion 5008 is moved away from the distal housing portion 5006, the distal element 3004 moves inwardly, although the distal element can be moved in other directions for other applications.

The housing 5004 of the handle assembly 3052 can be operated to move proximally and/or distally the control wire 4068. To pull the control wire 4068 proximally and reduce the diameter of the distal element 3004, the proximal housing portion 5008 can be moved away from the distal housing portion 5006.

In the illustrated embodiment of FIG. 43, the proximal housing portion 5008 is slidably mounted to the distal housing portion 5006.

The proximal housing portion 5008 has a tubular flange 5028 that slides over a tubular flange 5030 of the distal housing portion 5006. In the position illustrated in FIG. 30A, the housing 5004 is positioned such that the distal element 3004 is at rest. The proximal housing portion 5008 can be moved proximally relative to the distal housing portion 5006 to pull on the control wire 4068, which is connected to a pin 5034 mounted to an interior portion of the proximal housing portion 5008. The tensioned control wire 4068 can change the shape of the distal element 3004.

With continued reference to FIG. 43, the control system 3060 comprises knobs 5000, 5002 for actuating the shaft assembly 3050 to locate the distal element 3004. The illustrated control system 3060 comprises a drive assembly 5007 connected to the knobs 5000, 5002 and the first and the second pull wires 4032, 4034. The knobs 5000, 5002 are disposed within elongated slots 5010, 5012, respectively. The elongated slots 5010, 5012 are positioned at opposing sides of the distal housing portion 5006. The knobs 5010, 5012 can be moved along the elongated slots 5010, 5012, respectively, to operate the drive assembly 5008.

In the illustrated embodiment, the knob 5000 is at a distal end of the elongated slot 5010. The knob 5002 is at a proximal end of the elongated slot 5012. The drive assembly 5007 is configured so that the knob 5000 can be moved proximally while the knob 5002 is moved distally.

The drive assembly 5007 of FIG. 43 is a gear arrangement configured to actuate the pull wires 4032, 4034. The illustrated gear arrangement is in the form of a rack and pinion gear arrangement. The knob 5000 is connected to a first rack 5018 and the knob 5002 is connected to an opposing second rack 5020. A pinion is positioned between the first rack 5018 and the second rack 5020. As one of the knobs 5000, 5002 moves axially in one direction, the other one of the knobs 5000, 5002 moves in the opposite axial direction. Movement of the knobs 5000, 5002 causes corresponding movement of the pull wires 4032, 4034 to position the distal element 3004. Other exemplary drive arrangements 5008 can comprise one or more of the following: solenoids, stepper motors, controllers, gears, slides, and/or the like for controllably operating the pull wires 4032, 4034. It is contemplated that the control system 3060 can be mechanically driven, pneumatically driven, or electrically driven. For example, although not illustrated, the handle assembly 3052 can comprise a power source, such as a battery, that powers one or more drive motors that drive movable slides connected to the pull wires 4032, 4034.

The handle assembly 3052 includes an injection system 5038 for inflating the balloon 3007 of the distal element 3004. The injection system 5038 comprises an inflation port 5040 and an outlet port 5044. The inflation port 5040 of the injection system 5038 is positioned at the proximal end of the proximal housing portion 5008. The inflation port 5040 is in fluid communication with the delivery lumen 4060 of the shaft assembly 3050 via a delivery line 5042. The handle outlet port 5044 extends outwardly from the proximal housing portion 5008 and is preferably in fluid communication with the return lumen 4062. The inflation port 5040 and the handle outlet port 5044 each have a connector (e.g., a coupling member, a media source connector, etc.) suitable for connecting to fluid lines, syringe, or other delivery devices.

The connector can be a lure connector configured to permit coupling of an external media supply source, preferably a heated media supply source, to the distal element 3004. In addition, one or more valve structures can be placed a long the flow path of the inflation media. For example, one or more one-way valves can be positioned along the flow path within the catheter system 3020 to prevent backflow of the heated media. The amount of media within the distal element 3004 can be adjusted by selectively opening and closing the valves positioned along the flow path. Of course, the pressure within the distal element 3004 may be increased or decreased by increasing or decreasing, respectively, the pressure of the media delivered by an external fluid source to the handle assembly 3050.

In operation, access to the heart of a patient can be provided by various techniques and procedures so that the implantable device 3000 can be activated. For example, minimal invasive surgery techniques, laparoscopic procedures, and/or open surgical procedures can provide a convenient access path to the chambers of the heart for delivering the distal element 3004. In some embodiments, access to the heart can be provided through the chest of a patient, and may include, without limitation, conventional transthoracic surgical approaches, open and semi-open heart procedures, and port access techniques. Such surgical access and procedures preferably can utilize conventional surgical instruments for access and performing surgical procedures on the heart, for example, retractors, rib spreaders, trocars, laparoscopic instruments, forceps, cannulas, staplers, and the like. The implantable device 3000 can be activated in conjunction with another surgical procedure that provides access (e.g., mitral valve repair, bypass surgical procedures, etc.).

Generally, in an embodiment intended for access through the femoral vein and delivery to the left atrium, the catheter 302 can have a length within the range of from about 50 cm to about 150 cm, and a diameter of generally no more than about 5 French, 10 French, or 15 French. Those skilled in the art recognize that the catheter system can be configured and sized for various methods of activating the implantable device, as described below. The catheter system 3020 can be sized and configured so that it can be delivered using, for example, conventional transthoracic surgical, minimally invasive, or port access approaches. In view of the present disclosure, further dimensions and physical characteristics of catheters for navigation to particular sites within the body are well understood in the art.

In the illustrated embodiment of FIG. 29, the catheter system 3020 is delivered percutaneously into the heart. A guiding sheath can be placed in the vasculature system of the patient and used to guide the catheter system 3020 and its distal element 3004 to a desired deployment site.

In some embodiments, a guide wire is used to gain access through the superior or inferior vena cava, for example, through groin access for delivery through the inferior vena cava. The guiding sheath can be advanced over the guide wire and into the inferior vena cava 3022 shown in FIG. 29. The distal end of the guiding sheath can be passed through the right atrium and towards the septum 6000. Once the distal end of the guiding sheath is positioned proximate to the septum 6000, a needle or piercing member is preferably advanced through the guiding sheath and used to puncture the fossa ovalis or other portion of the septum. In some embodiments, the guiding sheath is dimensioned and sized to pass through the fossa ovalis without requiring a puncturing device. That is, the guiding sheath can pass through the natural anatomical structure of the fossa ovalis into the left atrium.

As shown in FIG. 44, the guiding sheath 6004 can be positioned through the inferior vena cava 3022 through the right atrium and a septal hole 6008. When the guiding sheath 6004 is position within the heart 3006, the catheter system 3020 can be advanced distally, as indicated by the arrow 6010, through the guiding sheath 6004. As the catheter system 3020 is advanced through the guiding sheath 6004, the distal element 3004 is somewhat straight. Thus, the distal element 3004 can be delivered through a low profile delivery sheath 6004 and can flex as it is advanced distally.

The catheter system 3020 can be advanced until the distal element 3004 passes out of an opening 6012 of the guiding sheath 604. Preferably, the distal element 3004 is in a generally collapsed state (e.g., a deflated state) as it is delivered through the guiding sheath 6004 for a low profile configuration.

As the distal element 3004 passes out of the opening 6012 of the guiding sheath 6004, the distal element 3004 can assume its at-rest configuration. As shown in FIG. 45, the distal element 3004 assumes a somewhat curved configuration as it extends out of the opening 6012. Of course, the catheter system 3020 can be twisted and rotated within the guiding sheath 6004 to position the distal element 3004. In some embodiments, the core 3074 comprises a superelastic material, although other materials can be used, such as polymers, metals, and the like. In some embodiments, the superelastic comprises superelastic Nitinol. The nitinol can be stressed-induced martensite. Advantageously, superelastic materials allow for large deformations without substantial plastic deformation. Thus, the core 3074 can be moved repeatedly to many different positions and configured with substantially no plastic deformation, even though the core 374 undergoes large deformations.

In some embodiments, the tip 3069 of the distal element 3004 can be an atraumatic tip that is configured to slide through the lumen of the delivery sheath 6004. The atraumatic tip 3069 can limit or prevent significant damage to the inner tissue of the heart 3006.

As shown in FIG. 46, the distal element 3004 is positioned within the left atrium of the heart 3006. A technician can operate the control system 3060 of the hand assembly 3052 to steer the distal element 3004 to a desired position, preferably steering the distal element 3004 onto or proximate to the implantable device 3000. One of the knobs 5000, 5002 can be moved proximally while the other knob 5000, 5002 is moved distally so that one of the pull wires is retracted to rotate the illustrated distal element 3004 to a desired position illustrated in phantom 6016 in FIG. 46. The control system 3060 can be used to remove, replace, and/or reposition the distal element 3004 during the procedure. After the distal element 3004 is in the desired engagement position, as shown in FIG. 29, the distal element 3004 can deliver thermal energy to the implantable device 3000. Media can be injected through the inflation port 5040 and through the handle assembly 3052 to the shaft assembly 3050. The media may or may not be heated. Preferably, the media is heated to a threshold or target temperature before being delivered to the distal element 3004. The media can flow through the delivery lumen 4060 and ultimately out of the inlet port 3381 of the distal element 3004. The balloon member 3070 is inflated and heated as the heated media fills the chamber 3080 of the distal element 3004. The heat from the distal element 3004 can be transferred to the implantable device 3000, preferably being transferred at least until the implantable device 3000 is activated, thereby changing the shape of the implantable device 3000. During the heating process, the controls 3060 can be used to ensure that the distal element 3004 is properly aligned with the implantable device 3000.

The distal element 3004 can have one or more markers that advantageously assist in locating and positioning the distal element 3004 relative to the implantable device 3000. As described above, the implantable device 3000 can likewise have markers that assist in positioning process. In some embodiments, the distal element 3004 comprises one or more markers (e.g., radiopaque markers) that can be aligned with corresponding markers of the implantable device 3000. Any suitable markers or locators can be utilized.

The radiopaque markers can be made from material that is readily identified when the distal element 3004 is positioned within the heart 3006. For example, the radiopaque markers can comprise gold, tungsten, and/or other materials as is well known to those of ordinary skill in the art. The markers can be adhered, welded, soldered, glued, or otherwise incorporated into the distal element 3004 as desired. For example, FIG. 38 illustrates a distal element 4036 that comprises a plurality of bands 4037 that can be in the form of radiopaque markers that are visible under fluoroscopy. The markers can be evenly or unevenly spaced along the length of the distal element 3004. The markers of the implantable device 3000 can be positioned at any suitable location to aid in the positioning of the distal element 3004 relative to the implantable device 3000. It is contemplated that other visualization techniques can be employed. In some embodiments, for example, echocardiograph visualization, fluoroscopy visualization, imaging techniques, optics, and the like can be employed to help deliver and position the distal element 3004 as desired.

With reference again to FIG. 46, after the implantable device 3000 has been activated, the catheter system 3020 can be retracted or moved proximally relative to the guide sheath 6004. As the catheter system 3020 is pulled proximally through the guide sheath 6004, the distal element 3004 is straightened and slid through the opening 6012 and into the guide sheath 6004. The catheter system 3020 and the guide sheath 6004 can be withdrawn from the vasculature, preferably withdrawn without damaging the vasculature tissue.

FIG. 47 illustrates a catheter system 6100 that comprises a handle assembly 6102 connected to a shaft assembly 6104. A distal element 6106 is attached to a distal end 6108 of the shaft assembly 6104. The catheter system 6100 is generally similar to a catheter system 3020 of FIG. 29, except as further described in detail below.

The illustrated catheter system 6100 includes a control system 6112 for moving the distal element 6106. The control system 6112 comprises a knob 6114 that is axially moveable along a slot 6116 defined by the housing 6018 of the handle assembly 6102. When the knob 6114 is pulled proximally relative to the housing 6018, the distal element 6106 is moved from a first configuration towards a second configuration 6121 (shown in phantom). The knob can have preferential locations corresponding to various configurations of the distal element.

With respect to FIG. 48, the catheter system 6100 has an inflation port 6022 that can be used to inflate the distal element 6106. Media can be injected through the inflation port 6022 and through the catheter system 6100 into the distal element 6106 to inflate the balloon member. The heated media within the balloon member is in a generally static state and can be used to activate an implant, such as the implantable device 3000 described above.

The catheter systems described herein can be used to activate an implant during an open-heart procedure. For example, the catheter systems can be modified for delivery through a chest of a patient as an adjunct to another surgical procedure, such as valve leaflet repair, septum repair, and the like. The surgeon can manually guide the distal element of the catheter system through the chest of the patient and into a desired position within the heart. Of course, the catheter system can have a shaft assembly with a length that is generally less than the length of the shaft assembly of a catheter system for delivery through the superior or inferior vena cava. For example, the catheter system 6100 of FIG. 48 can be used during an open-heart procedure. The shaft assembly 6104 may have a length of about 100 centimeters or less, 80 centimeters or less, 50 centimeters or less, 30 centimeters or less, or even 20 centimeters or less. The catheter system 6100 may or may not be used with a guiding sheath. The guiding sheath can be a delivery sheath or other cannulated structure. In some embodiments, the catheter system 6100 is guided and placed manually within the patient's body without the use of a guide sheath.

FIG. 49 is a cross-sectional view of another embodiment of a distal element adapted to activate an implant. The distal element 6130 is illustrated in an inflated position. The distal element 6130 can be moved between an inflated position and a collapsed position.

The distal element 6130 comprises a wall 6132 and a chamber 6134 defined between the interior surface of the wall 6132 and a heating element 6136. Although not illustrated, the wall 6132 can engage and surround the heating element 6136 when the distal element 6130 is in the collapsed state. The element 6130 can be inflated by delivering material 6138 (e.g., media) to fill the chamber 6134. When no external force is applied to the distal element 6130, the heating element 6136 can be in the illustrated neutral position. Preferably, the heating element 6136 can move freely through the material 6138 so that the heating element 6136 is eccentrically positioned. The heating element 6136 can be located near a portion of the wall 6132 when the distal element 6130 presses against a surface, as shown in FIG. 50.

The wall 6132 can be a somewhat flexible membrane that is preferably thermally conductive to enhance heat transfer from the heating element 6136 to the implantable device. For example, the wall 6132 can comprise a membrane that is doped with a thermally conductive material, such as metallic particles and the like. The wall 6132 can be flexible to permit inflation within desired range of size. In some embodiments, the wall 6132 comprises carbon (e.g., carbon fibers) that is incorporated into the wall 6132. The types of materials and construction of the wall 6132 can be selected to accommodate for various working pressures, implant designs, and the like.

The material 6138 can form a layer between the heating element 6136 and the wall 6132. The material 6138 can be selected to achieve the desired heat flow from the heating element 6136 to the wall 6132. For example, in some embodiments, it may be desirable to have somewhat localized heating of the wall 6132. As shown in FIG. 50, the heating element 6136 can be eccentrically located and proximate to a portion of the wall 6032 which is nearest the implant 3000. It may be desirable to have the opposing portion of the wall 6132 at a lower temperature than the portion 6144 of the wall 6132 interposed between the heating element 6136 and the implant 3000. Thus, the heating element 6136 can provide localized heating for an efficient heating process that limits the amount of blood, surrounding the distal element 6130, that is heated. When the heating element 6136 is eccentrically positioned in the distal element 6130, heat from the distal element 6130 is directed to the implantantable device thereby minimizing heat losses. During delivery of the distal element 6130, the heating element 6136 is generally centrally located in the distal element 6130, as shown in FIG. 49. However, pressure applied the wall 6132 causes the heating element 6136 to be biased towards the applied pressure, which is preferably towards a surface of an implant.

The material 6138 can comprise a material that has a thermal conductivity that is equal to or less than the thermal conductivity of the material forming the wall 6132. When the heating element 6136 is proximate to the wall portion 6144, the material 6138 can provide thermal resistance to keep the temperature of the surface of the distal element 6130, which is not touching or proximate to the implant 3000, and relatively low temperatures. The material 6138 can be a gas, fluid, gel, flowable material, combination thereof, and the like. In some embodiments, the material 6138 comprises saline or water. The heating element 6130 can be moved closer to the wall 6132 to account for materials 6131 having a low thermal conductivity.

In operation, the distal element 6130 can be in a generally neutral position such that the wall 6132 is generally concentric with the heating element 6136. The material 6138 forms a generally uniform layer between the heating element 6136 and the wall 6132. In this position, the material 6138 forms a insulating layer that limits the amount of heat transfer between the heating element 6136 and the external fluid (e.g., blood) surrounding the distal element 6130. Additionally, the outer surface of the wall 6132 can have a generally uniform temperature.

To achieve heating, preferably localized heating, the heating element 6136 is biased, as shown in FIG. 50, towards the implantable device 3000. When an external force is applied to the distal element 6130, the heating element 6136 can be biased towards the applied thereto. In the illustrated embodiment of FIG. 50, the distal element 6130 can be pressed against the implant 3000 to move the heating element 6136 through the material 6138 towards the wall portion 6144. The heating element 6136 can contact, or is proximate to, the portion 6144. The heating element 6136 can be activated to generate heat that is transferred through the wall portion 6144 and to the implant 3000. The heating element 6136 can be heated at any time during the procedure. For example, the heating element 6136 can be heated before or after the distal element 6130 is disposed in the left atrium. While heat is being delivered to the implant 3000, the material 6138 can insulate and limit the amount of heat transferred from the heating element 6136 to the other portions of the wall 6132, thus minimizing thermal losses.

FIG. 51 illustrates a portion of the distal element 6130 having a biased heating element. The distal element 6130 can have one or more pull wires that are used in combination to move the heating element 6136 to a desired position relative to the wall 6132. As shown in FIG. 51, the distal element 6130 is curved such that the heating element is proximate to or contacts a first surface 6150. Thus, the heating element 6136 can provide localized heating of the first surface 6150. To bias the heating element 6136 in this manner, the pull wire 6152 can be pulled in the proximal direction thereby causing the distal element 6130 to curve in the direction indicated by the arrow 6154 from the neutral position 6161 (FIG. 53). When the distal element 6130 is in the neutral position, the heating element 6136 can be generally centrally positioned within the tubular wall 6132.

Preferably, the first surface 6150 can be placed in a contact with the implant 3000 to rapidly heat the implant while minimizing heat losses to the surrounding blood.

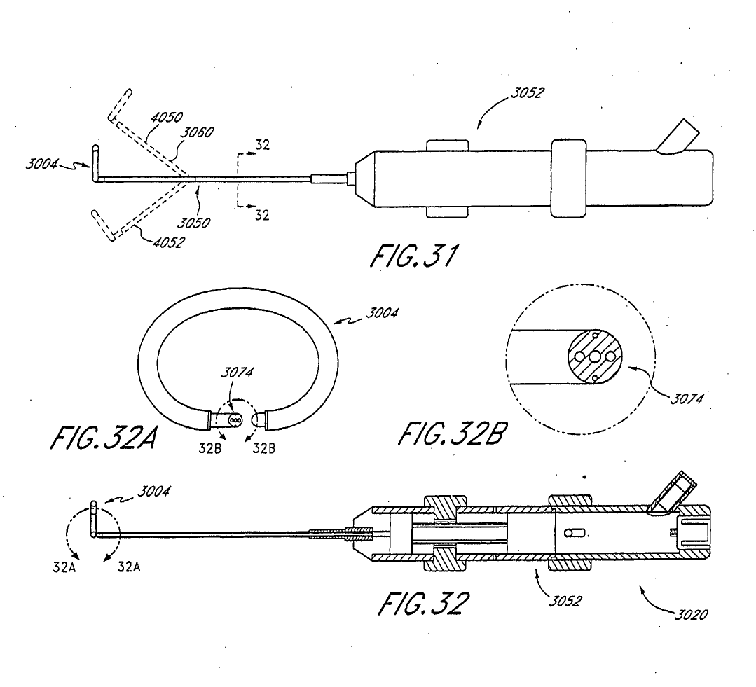

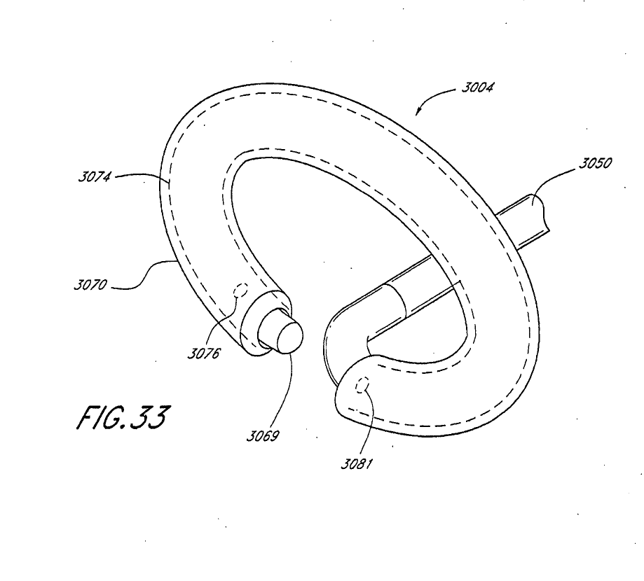

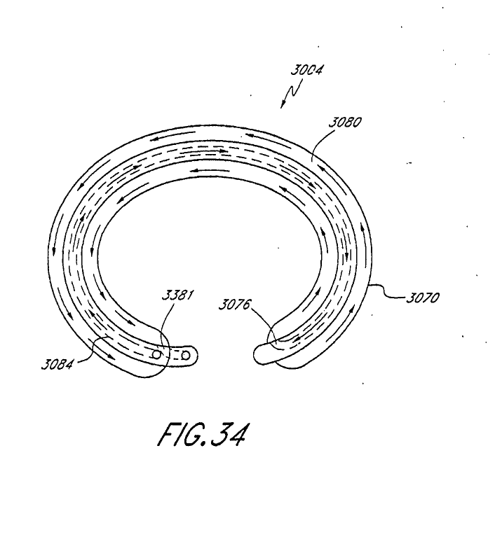

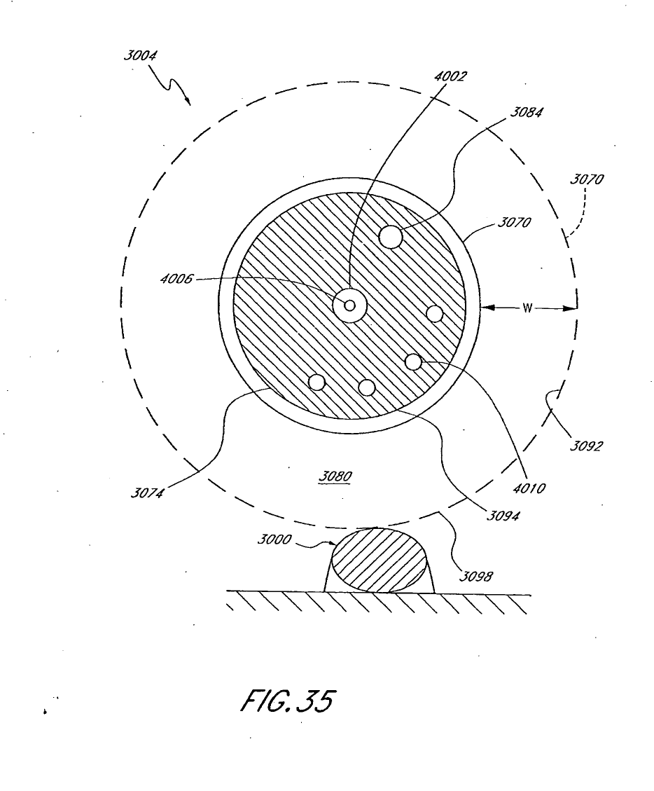

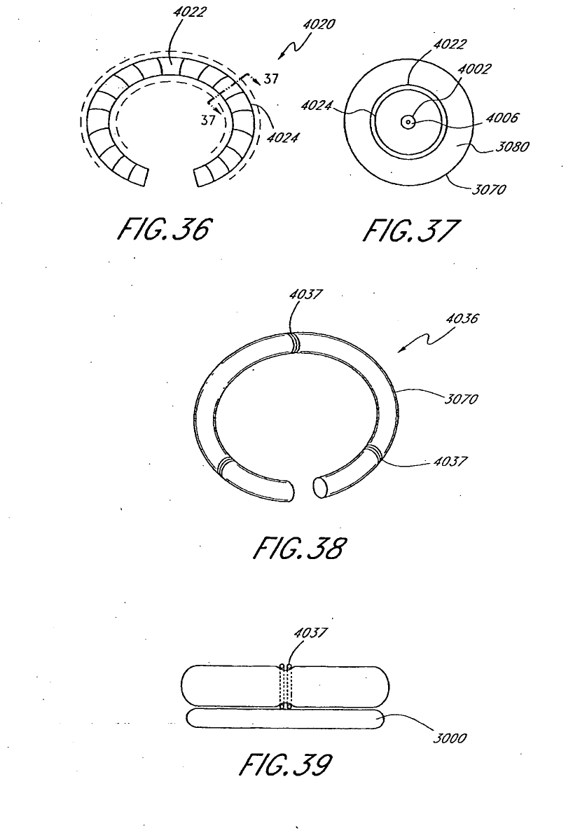

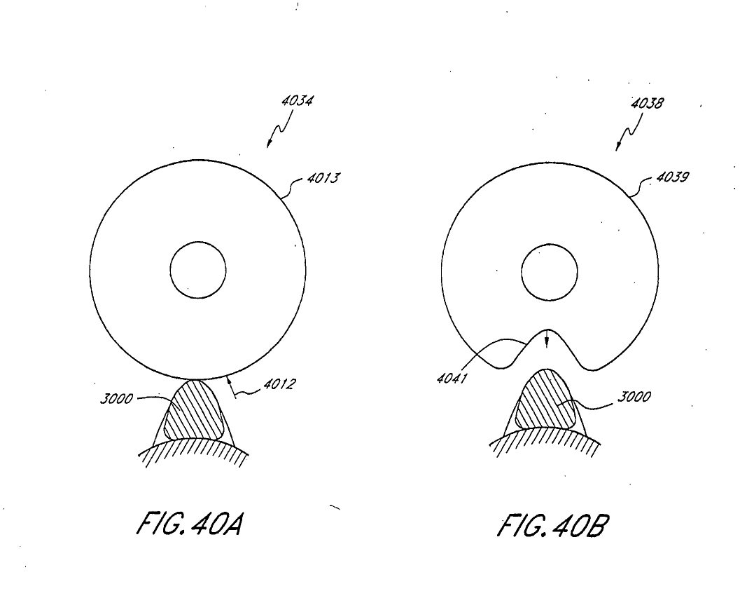

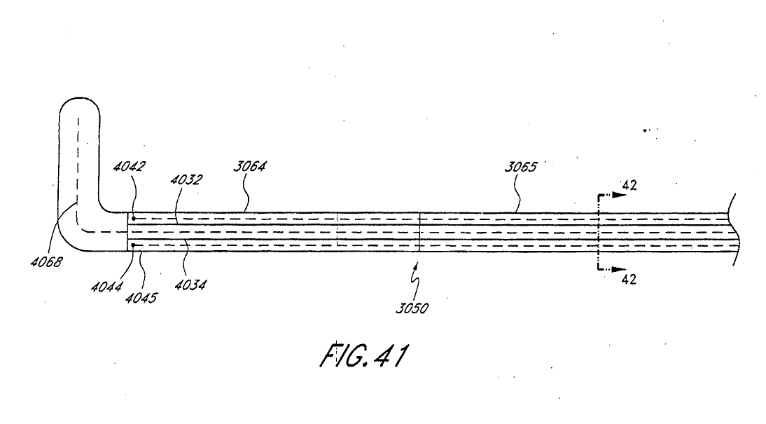

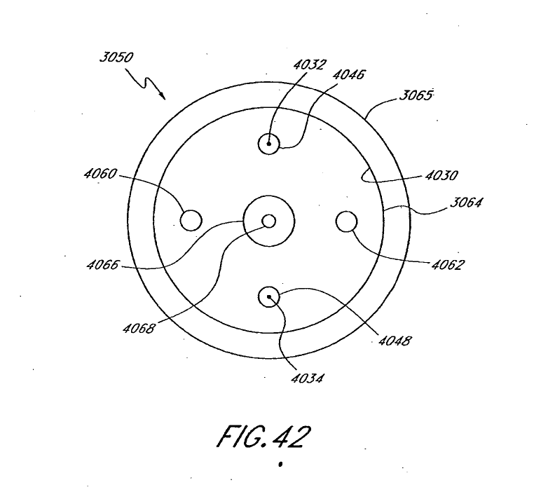

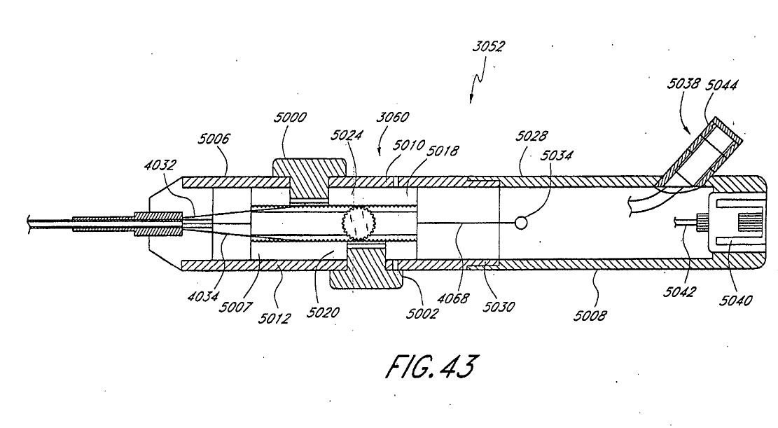

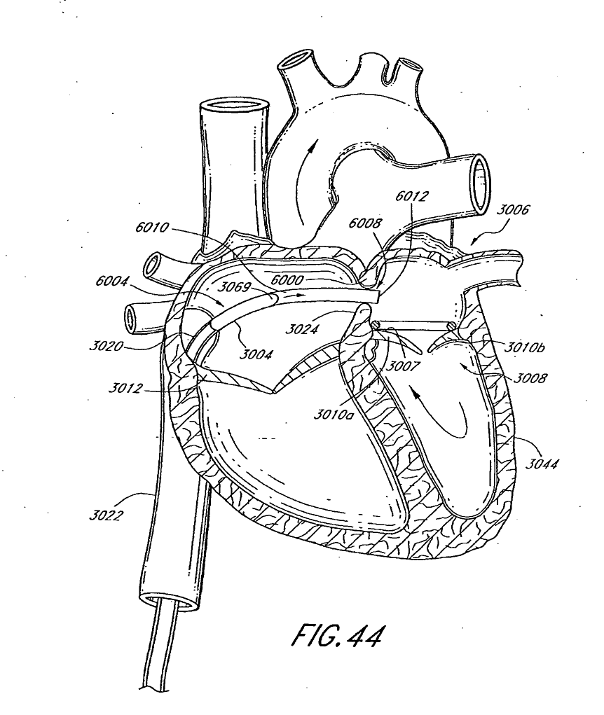

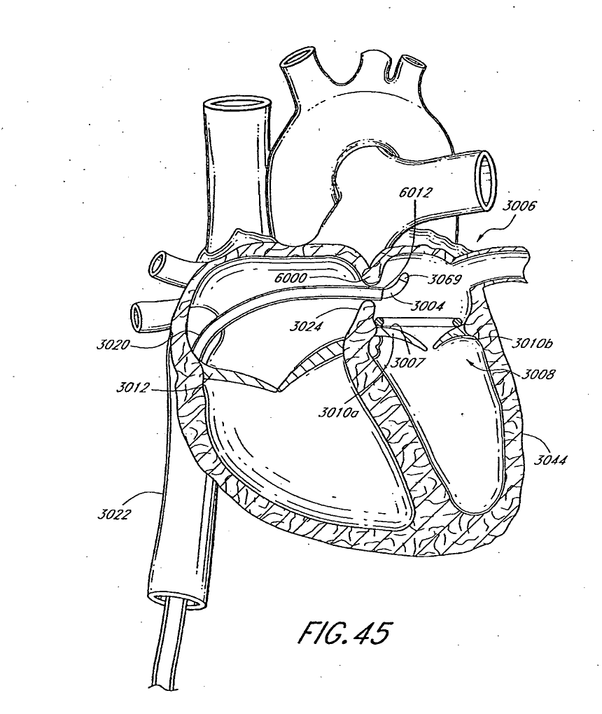

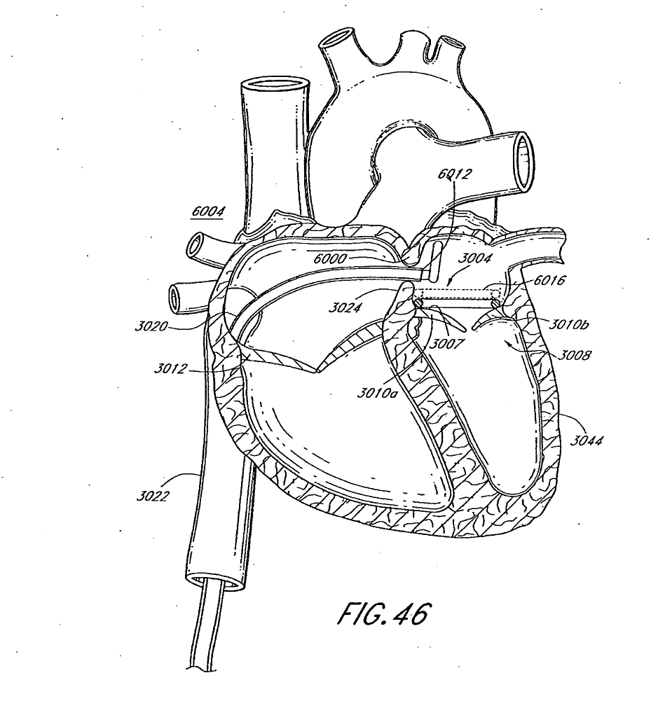

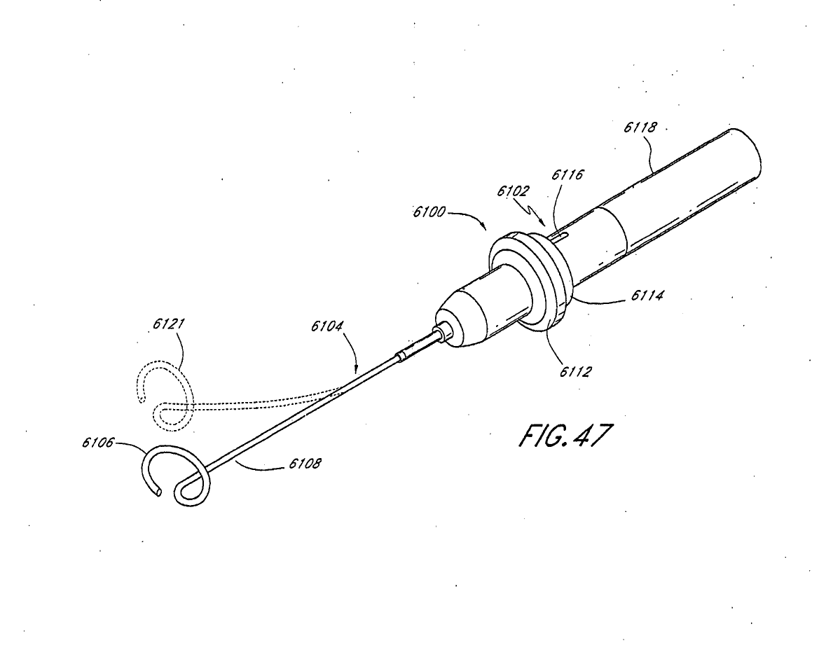

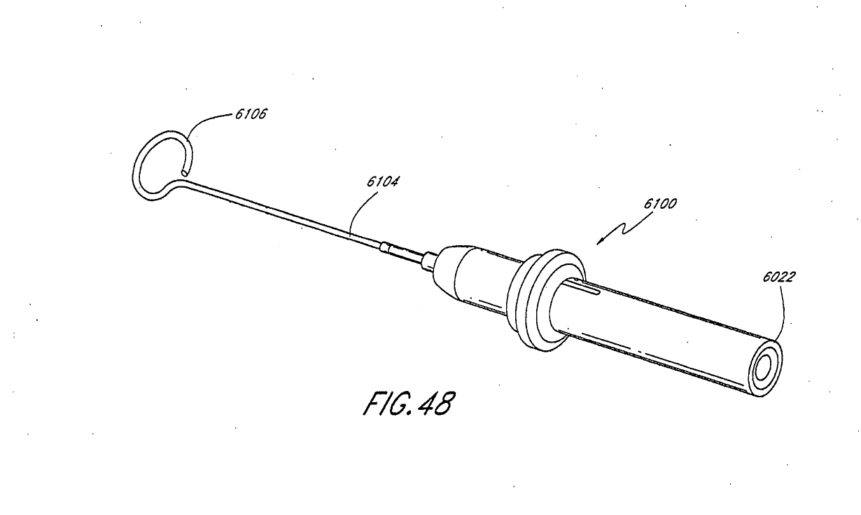

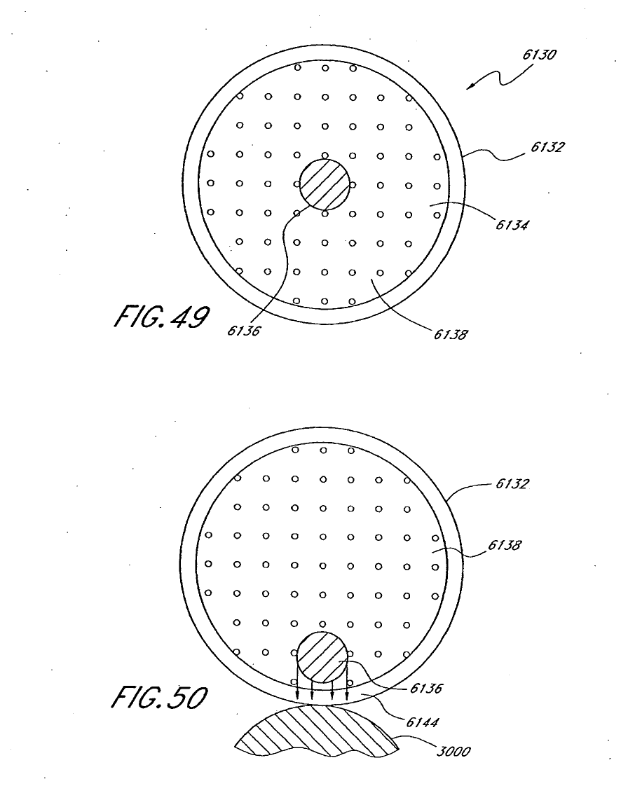

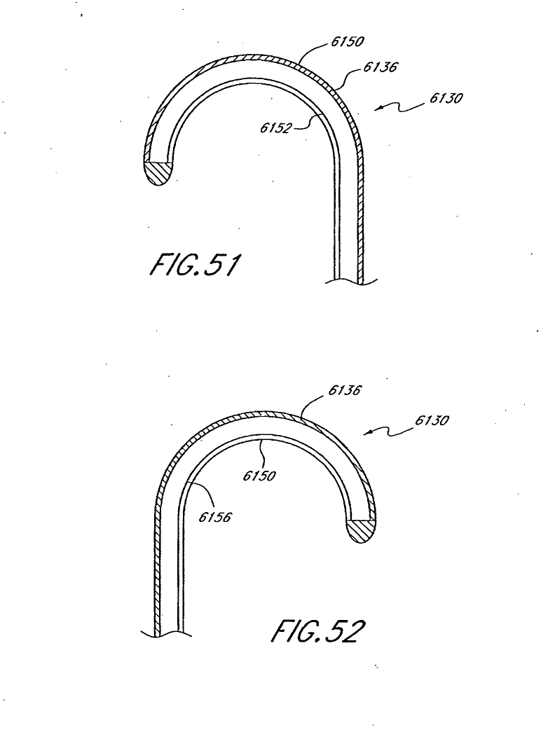

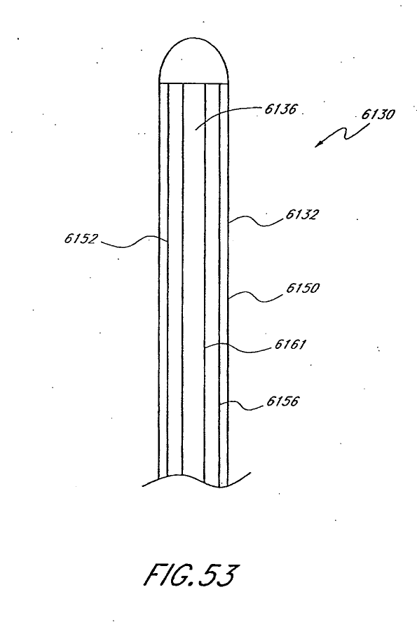

With respect to FIG. 52, the pull wire 6156 of the distal element 6130 can be pulled proximally to bias the distal element 6130 in the direction indicated by the arrow 6160 from the neutral position 6161. Thus, the heating element 6136 is proximal to or contacts the second surface 6162. Any number of pull wires can be used to actuate the distal member 6130 in any of a number of directions. For example, three pull wires can be used to move the tip in any desired direction. The heating element 6136 and/or the conducting wall 6132 can provide a biasing force in the opposite direction as the deflection of the distal element 6130.

The catheter system can also be used to activate devices implanted at other locations. In some embodiments, the catheter systems described herein can be used to activate an implantable device positioned within the coronary sinus, for example.

The catheter system can be positioned in the left atrium and used to deliver heat through the wall of the heart to the implant. For example, the distal element can be on one side of the heart wall and the device can be positioned within the coronary sinus on the other side of the heart wall. Heat can be emitted from the distal element through the tissue of the heart and eventually to the implantable device. Once the implantable device has been elevated to target temperature, the implantable device is activated and can change its configuration. Alternatively, the distal element can be externally positioned (e.g., outside of the heart) and placed against the coronary sinus. Once again, heat from the distal element can be used activate the implanted device positioned within the coronary sinus. Thus, the catheter system can be used to activate an implantable (e.g., adjustable annuloplasty device) that is positioned within the coronary sinus to treat a patient's heart.

All patents and publications mentioned herein are hereby incorporated by reference in their entireties. Except as further described herein, the embodiments, features, systems, devices, materials, methods and techniques described herein may, in some embodiments, be similar to any one or more of the embodiments, features, systems, devices, materials, methods and techniques described in U.S. application Ser. No. 11/181,686, filed Jul. 14, 2005, U.S. application Ser. No. 11/124,364, filed May 6, 2005, U.S. application Ser. No. 11/111,682, filed Apr. 21, 2005, and U.S. application Ser. No. 11/123,874, filed May 6, 2005. In addition, the embodiments, features, systems, devices, materials, methods and techniques described herein may, in certain embodiments, be applied to or used in connection with any one or more of the embodiments, features, systems, devices, materials, methods and techniques disclosed in the above-mentioned U.S. application Ser. No. 11/181,686, filed Jul. 14, 2005, U.S. application Ser. No. 11/124,364, filed May 6, 2005, U.S. application Ser. No. 11/111,682, filed Apr. 21, 2005, and U.S. application Ser. No. 11/123,874, filed May 6, 2005.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A catheter system for percutaneously activating an adjustable annuloplasty device, the catheter system comprising:
a handle assembly;
a shaft assembly having at least one fluid lumen; and
a distal element, the shaft assembly extending between the handle assembly and the distal element, the distal element being in fluid communication with the handle assembly via the at least one fluid lumen, the distal element comprising:
an elongated core having a first port;
an expandable member, the core extending through the expandable member, the expandable member being movable between a collapsed position and an inflated position, wherein the distal element has a preset shape, in the inflated position, having a long axis that is curvilinear, and a surface of the distal element extending along the long axis is configured to conform to a curvilinear surface of the annuloplasty device, said annuloplasty device surface extending along a circumference of the annuloplasty device.

2. The catheter system of claim 1, wherein the annuloplasty device comprises a ring, and the circumference of the annuloplasty device is a circumference of the ring.

3. The catheter system of claim 1, further comprising a long axis of the annuloplasty device that is concentric with the circumference of the annuloplasty device, and wherein the surface of the distal element is configured to conform to a surface of the annuloplasty device that extends along the long axis of the annuloplasty device.

4. The catheter system of claim 1, further comprising a chamber being defined between the core and the expandable member when the expandable member occupies the inflated position, wherein the chamber is in fluid communication with the at least one fluid lumen via the first port.

5. The catheter system of claim 1, wherein the handle assembly is coupled to a proximal end of the shaft assembly, the distal element is coupled to a distal end of the shaft assembly, the handle assembly has an insertion port in fluid communication with the at least one fluid lumen.

6. The catheter system of claim 1, wherein the handle assembly comprises a control system, the control system being movable between a first actuation position and a second actuation position, the shaft assembly occupies a first position when the control system is in the first actuation position, and the shaft assembly occupies a second position when the control system is in the second actuation position.

7. The catheter system of claim 1, wherein the handle assembly comprises a control system, the control system movable between a first sizing position and a second sizing position, the distal element has a first configuration when the control system is in the first sizing position, and the distal element has a second configuration when the control system is in the second sizing position.

8. The catheter system of claim 1, wherein the expandable member is a balloon member and the core extends therethrough.

9. The catheter system of claim 1, wherein the distal element has a generally annular configuration.

10. The catheter system of claim 9, wherein the distal element has an open oval shape.

11. A system for activating a device implanted in a patient, the system comprising:
a handle assembly;
a flexible, steerable shaft assembly; and
a distal element being expandable between a first position and a second position, the distal element being dimensioned so as to have a curvilinear long axis that matches a curvilinear long axis of an annuloplasty device implanted at or near a valve in a patient's heart.

12. The device of claim 11, wherein a substantial portion of the long axis of the distal element has generally the same shape as a substantial portion of the long axis of the annuloplasty device.

13. The device of claim 11, wherein the shaft assembly has delivery lumen and a return lumen in fluid communication with a fluid chamber distal element.

14. The device of claim 11, wherein a plurality of pull wires extend through the flexible shaft assembly, the flexible shaft assembly being a movable between a first position and a second position when at least one of the pull wires is actuated.

15. The device of claim 11, further comprising a control wire extending through a control lumen of the flexible shaft assembly, and actuation of the control wire moves the distal element between a first configuration and a second configuration.

16. A method for activating an implantable device, the method comprising:
providing a catheter assembly having a distal element thereon;
positioning the distal element within an atrium of a heart of a patient proximal to an annuloplasty device located at or near a valve of said heart; and
delivering sufficient energy from said distal element to said annuloplasty device to change a configuration of said annuloplasty device.

17. The method of claim 16, wherein said annuloplasty device has a first size of a dimension of said device in a first configuration and a second size of said dimension of said device in a second configuration, the change in configuration comprises moving said annuloplasty device from said first configuration to said second configuration, wherein said second size is less than said first size.

18. The method of claim 16, wherein the distal element has a preset shape, in the inflated position, having a long axis that is curvilinear, and a surface of the distal element extends along the long axis is configured to conform to a curvilinear surface of the annuloplasty device, said annuloplasty device surface extending along a circumference of the annuloplasty device.

19. The method of claim 16, wherein said annuloplasty device is in a heart having a first end-diastolic volume of a ventricle, and after said change in configuration of said annuloplasty device, said ventricle has a second end-diastolic volume, said second end-diastolic volume being less than said first end-diastolic volume.

20. The method of claim 19, wherein said annuloplasty device has a first size of a dimension of said device in a first configuration and a second size of said dimension of said device in a second configuration, the change in configuration comprises moving said annuloplasty device from said first configuration to said second configuration, wherein said second size is less than said first size.

21. The method of claim 16, wherein the delivering energy from the distal element comprises passing a heated media through the distal element.

22. The method of claim 16, wherein the device implanted in the heart comprises an annuloplasty ring moveable between a first configuration and a second configuration by the application of energy to the implanted device.

23. The method of claim 22, wherein the applying of energy comprises heating the annuloplasty ring to a predetermined temperature, the annuloplasty ring comprises shape memory material, the shape memory material substantially changes shape when heated to the predetermined temperature.

24. The method of claim 23, wherein heated fluid is passed through the distal element, and heat from the heated media increases a temperature of the implanted device to a predetermined activation temperature.

25. The method of claim 16, further comprising:
providing a steerable flexible shaft assembly, the distal element being connected to a distal end of the flexible shaft assembly;
placing the flexible shaft assembly through the a right atrium, septum, and into the left atrium of the heart; and
placing the distal element in operative engagement with the implanted device, wherein the implanted device affects functioning of a mitral valve.

26. The method of claim 25, further comprising activating the implanted device with the distal element, the implanted device moves leaflets of the mitral valve when the implanted device is activated.
